(19) United States
(12) Patent Application Publication
(10) Pub. No.: US 2023/0116380 A1
(43) Pub. Date: Apr. 13, 2023

(54) LONG-ACTING GM-CSF AND METHODS OF USE

(71) Applicant: The Scripps Research Institute, La Jolla, CA (US)

(72) Inventors: Sean JOSEPH, San Diego, CA (US); Lei LEI, San Diego, CA (US); Weijun SHEN, San Diego, CA (US); Feng WANG, Carlsbad, CA (US); Peter SCHULTZ, La Jolla, CA (US)

(21) Appl. No.: 17/799,246

(22) PCT Filed: Feb. 11, 2021
(86) PCT No.: PCT/US2021/017684

§ 371 Date: Aug. 11, 2022

(30) Foreign Application Priority Data
Feb. 12, 2020 (CN) PCT/CN2020/074834

(51) Int. Cl. C07K 14/535; C07K 16/10; A61P 25/28

(52) U.S. Cl. CPC C07K 14/535; C07K 16/1027; A61P 25/28; A61K 2039/505

(57) ABSTRACT

Disclosed herein are granulocyte-macrophage colony-stimulating factor (GM-CSF) peptides and compositions comprising GM-CSF peptides. These molecules may be useful for the treatment of neurological diseases or conditions.

### CROSS-REFERENCE

This application claims the benefit of International Application No. PCT/CN2020/074834, filed Feb. 12, 2020, which application is incorporated herein by reference in its entirety.

### BACKGROUND OF THE INVENTION

Parkinson's disease (PD) is a progressive neurodegenerative disease associated with substantial morbidity, increased mortality, and particularly high economic burden.

### SUMMARY OF THE INVENTION

The progression of Parkinson's disease (PD) and other neurodegenerative conditions is linked to inflammation. Preclinically, GM-CSF treatment modulates innate microglial immunity and increases regulatory T cells (T_{reg}) that transmigrate from the periphery into the brain, resulting in anti-inflammatory and neuroprotective responses. In one aspect, provided herein are GM-CSF molecules for the treatment of neurodegenerative conditions and/or inflammation-related conditions. In some embodiments, a GM-CSF molecule provided here is used for the treatment of one or more of Parkinson's disease (PD), amyotrophic lateral sclerosis (ALS), Alzheimer's disease (AD), acute radiation syndrome, traumatic brain injury, cancer, and Crohn's disease (CD).

GM-CSF displays limited bioavailability and a short-half life, such that GM-CSF peptide therapeutics have required high dosing and daily administration. Mild-to-moderate adverse events have been experienced with daily GM-CSF treatment, including injection site reactions, elevation of WBC counts, and bone pain. In one aspect, provided herein are long-acting GM-CSF molecules comprising a GM-CSF connected to a scaffold to increase the half-life of GM-CSF. Exemplary scaffolds comprise an antibody variable domain. In addition, various long-acting GM-CSF molecules provided herein have increased bioavailability of GM-CSF as compared to GM-CSF peptide alone. Various long-acting GM-CSF molecules provided herein may be administered with a frequency between about once every 7 days to about once a month, for instance, at a frequency of about once every two weeks.

In one aspect, provided herein is a composition comprising a first polypeptide comprising a granulocyte macrophage colony stimulating factor (GM-CSF) and a second polypeptide comprising a sequence at least 98% identical to SEQ ID NO: 2. In some embodiments, GM-CSF comprises a sequence at least about 85%, 86%, 87%, 88%, 89%, 90%, 91%, 92%, 93%, 94%, 95%, 96%, 97%, 98%, or 99% identical to SEQ ID NO: 16. In some embodiments, GM-CSF comprises a sequence at least about 85%, 86%, 87%, 88%, 89%, 90%, 91%, 92%, 93%, 94%, 95%, 96%, 97%, 98%, or 99% identical to SEQ ID NO: 77. In some embodiments, the GM-CSF comprises human GM-CSF or murine GM-CSF. In some embodiments, the first polypeptide comprises a modified light chain of an antibody variable region. In some embodiments, the modified light chain of the antibody variable domain comprises the GM-CSF positioned between a first amino acid sequence of the antibody variable region and a second amino acid sequence of the antibody variable region. In some embodiments, the first amino acid sequence comprises a sequence at least about 90%, 91%, 92%, 93%, 94%, 95%, 96%, 97%, 98%, or 99% identical to SEQ ID NO: 14. In some embodiments, the first amino acid sequence comprises SEQ ID NO: 14. In some embodiments, the second amino acid sequence comprises a sequence at least about 90%, 91%, 92%, 93%, 94%, 95%, 96%, 97%, 98%, or 99% identical to SEQ ID NO: 15. In some embodiments, the second amino acid sequence comprises SEQ ID NO: 15. In some embodiments, the GM-CSF is positioned within a complementarity determining region (CDR) of the modified light chain. In some embodiments, the GM-CSF is position within light chain CDR1, CDR2, or CDR3. In some embodiments, the GM-CSF is positioned within light chain CDR3. In some embodiments, the modified light chain is modified from a variable light chain comprising SEQ ID NO: 17. In some embodiments, the first polypeptide further comprises a first linker peptide. In some embodiments, the first linker peptide comprises SEQ ID NO: 10. In some embodiments, the first linker peptide comprises SEQ ID NO: 8. In some embodiments, the first linker peptide comprises SEQ ID NO: 11. In some embodiments, the first linker peptide comprises a sequence at least about 90%, 91%, 92%, 93%, 94%, 95%, 96%, 97%, 98%, 99% or 100% identical to SEQ ID NO: 12. In some embodiments, the first polypeptide further comprises a second linker peptide. In some embodiments, the second linker peptide comprises SEQ ID NO: 10. In some embodiments, the second linker peptide comprises SEQ ID NO: 9. In some embodiments, the second linker peptide comprises SEQ ID NO: 11. In some embodiments, the second linker peptide comprises a sequence at least about 90%, 91%, 92%, 93%, 94%, 95%, 96%, 97%, 98%, 99%, or 100% identical to SEQ ID NO: 13. In some embodiments, the first polypeptide comprises a sequence at least about 90%, 91%, 92%, 93%, 94%, 95%, 96%, 97%, 98%, 99%, or 100% identical to SEQ ID NO: 18. In some embodiments, the first polypeptide comprises a sequence at least about 90%, 91%, 92%, 93%, 94%, 95%, 96%, 97%, 98%, 99%, or 100% identical to SEQ ID NO: 6. In some embodiments, the first polypeptide comprises a sequence at least about 90%, 91%, 92%, 93%, 94%, 95%, 96%, 97%, 98%, 99%, or 100% identical to SEQ ID NO: 7. In some embodiments, the first polypeptide comprises a sequence at least about 90%, 91%, 92%, 93%, 94%, 95%, 96%, 97%, 98%, 99%, or 100% identical to SEQ ID NO: 5. In some embodiments, the second polypeptide comprises a heavy chain of an antibody variable region. In some embodiments, the second polypeptide comprises SEQ ID NO: 2. In some embodiments, the second polypeptide further comprises a sequence at least about 90%, 91%, 92%, 93%, 94%, 95%, 96%, 97%, 98%, 99%, or 100% identical to SEQ ID NO: 4. In some embodiments, the second polypeptide comprises a sequence at least about 90%, 91%, 92%, 93%, 94%, 95%, 96%, 97%, 98%, 99%, or 100% identical to SEQ ID NO: 1. In some embodiments, the first polypeptide and the second polypeptide are connected via one or more disulfide bonds. In some embodiments, the first polypeptide and the second polypeptide form an antibody variable domain. In some embodiments, the antibody variable domain does not bind to an antigen with an equilibrium dissociation constant (KD) lower than about 10-2 M, 10-3 M, or 10-4 M. In some embodiments, the antibody variable domain comprises a modified palivizumab variable domain. In some embodiments, the modified palivizumab variable domain comprises a heavy chain CDR1 comprising SEQ ID NO: 19. In some embodiments, the modified palivizumab variable domain comprises a heavy chain CDR2 comprising SEQ ID NO: 20. In some embodiments, the modified palivizumab variable domain comprises a heavy chain CDR3 comprising SEQ ID NO: 21. In some embodiments, the modified palivizumab variable domain comprises a light chain CDR1 comprising SEQ ID NO: 22. In some embodiments, the modified palivizumab variable domain comprises a light chain CDR2 comprising SEQ ID NO: 23. In some embodiments, the modified palivizumab variable domain comprises a light chain CDR3 comprising SEQ ID NO: 24, 77 or 16. In some embodiments, the modified palivizumab variable domain does not bind to Respiratory Syncytial Virus (RSV) with a KD lower than about 10-2 M, 10-3 M, or 10-4 M. In some embodiments, the composition further comprises a Fc region comprising reduced effector function as compared to human IgG1. In some embodiments, the human IgG1 comprises SEQ ID NO: 25. In some embodiments, the reduced effector function comprises reduced antibody-dependent cellular cytotoxicity (ADCC). In some embodiments, the reduced effector function comprises reduced complement dependent cytotoxicity (CDC). In some embodiments, the first polypeptide further comprises a sequence at least about 90%, 91%, 92%, 93%, 94%, 95%, 96%, 97%, 98%, 99%, or 100% identical to SEQ ID NO: 3; and/or the first polypeptide comprises a Fc region comprising a human IgG1 comprising E233P, L234V, L235A, ΔG236, A327G, A330S, P331S, per Kabat numbering.

In one aspect, provided herein is a composition comprising an antibody variable domain comprising a light chain sequence comprising a first polypeptide comprising a sequence at least about 90% identical to SEQ ID NO: 6, and a heavy chain sequence comprising a second polypeptide comprising a sequence at least about 90% identical to SEQ ID NO: 2. In some embodiments, the first polypeptide comprises a sequence at least about 91%, 92%, 93%, 94%, 95%, 96%, 97%, 98%, 99%, or 100% identical to SEQ ID NO: 6. In some embodiments, the second polypeptide comprises a sequence at least about 91%, 92%, 93%, 94%, 95%, 96%, 97%, 98%, 99%, or 100% identical to SEQ ID NO: 2. In some embodiments, the composition comprise GM-CSF. In some embodiments, the GM-CSF is human GM-CSF or murine GM-CSF. In some embodiments, GM-CSF comprises a sequence at least about 85%, 86%, 87%, 88%, 89%, 90%, 91%, 92%, 93%, 94%, 95%, 96%, 97%, 98%, or 99% identical to SEQ ID NO: 16. In some embodiments, GM-CSF comprises a sequence at least about 85%, 86%, 87%, 88%, 89%, 90%, 91%, 92%, 93%, 94%, 95%, 96%, 97%, 98%, or 99% identical to SEQ ID NO: 77. In some embodiments, the light chain comprises a sequence at least about 90%, 91%, 92%, 93%, 94%, 95%, 96%, 97%, 98%, 99%, or 100% identical to SEQ ID NO: 7. In some embodiments, the light chain comprises a sequence at least about 90%, 91%, 92%, 93%, 94%, 95%, 96%, 97%, 98%, 99%, or 100% identical to SEQ ID NO: 5. In some embodiments, the heavy chain comprises a sequence at least about 90%, 91%, 92%, 93%, 94%, 95%, 96%, 97%, 98%, 99%, or 100% identical to SEQ ID NO: 4. In some embodiments, the heavy chain comprises a sequence at least about 90%, 91%, 92%, 93%, 94%, 95%, 96%, 97%, 98%, 99%, or 100% identical to SEQ ID NO: 1. In some embodiments, the composition further comprises a Fc region comprising reduced effector function as compared to human IgG1. In some embodiments, the human IgG1 comprises SEQ ID NO: 25. In some embodiments, the reduced effector function comprises reduced antibody-dependent cellular cytotoxicity (ADCC). In some embodiments, the reduced effector function comprises reduced complement dependent cytotoxicity (CDC). In some embodiments, the heavy chain further comprises a sequence at least about 90%, 91%, 92%, 93%, 94%, 95%, 96%, 97%, 98%, 99%, or 100% identical to SEQ ID NO: 3; and/or the heavy chain comprises a Fc region comprising a human IgG1 comprising E233P, L234V, L235A, ΔG236, A327G, A330S, P331S, per Kabat numbering.

In one aspect, provided herein is a composition comprising an antibody variable domain comprising a light chain sequence comprising a sequence at least about 90% identical to SEQ ID NO: 26 (DIQMTQSPSTLSASVGDRVTITCKCQLSVGYMHWYQQKPGKAPKLLIYDTSKLASGVPSRFSGSGSG TEFTLTISSLQPDDFATYYCFQGS[X1]PFTFGGGTKLEIKR), wherein the light chain sequence comprises X1 and X1 comprises GM-CSF; and a heavy chain sequence comprising a sequence at least about 90% identical to SEQ ID NO: 2. In some embodiments, the GM-CSF is human GM-CSF or murine GM-CSF. In some embodiments, GM-CSF comprises a sequence at least about 85%, 86%, 87%, 88%, 89%, 90%, 91%, 92%, 93%, 94%, 95%, 96%, 97%, 98%, or 99% identical to SEQ ID NO: 16. In some embodiments, GM-CSF comprises a sequence at least about 85%, 86%, 87%, 88%, 89%, 90%, 91%, 92%, 93%, 94%, 95%, 96%, 97%, 98%, or 99% identical to SEQ ID NO: 77. In some embodiments, the light chain sequence comprises a sequence at least about 91%, 92%, 93%, 94%, 95%, 96%, 97%, 98%, 99%, or 100% identical to SEQ ID NO: 26. In some embodiments, the light chain sequence comprises a sequence at least about 90%, 91%, 92%, 93%, 94%, 95%, 96%, 97%, 98%, 99%, or 100% identical to SEQ ID NO: 27 (DIQMTQSPSTLSASVGDRVTITCKCQLSVGYMHWYQQKPGKAPKLLIYDTSKLASGVPSRFSGSGSG TEFTLTISSLQPDDFATYYCFQGSGGSGAKLAALKAKLAALKGGGGS[X2]GGGGSELAALEAELAAL EAGGSGPFTFGGGTKLEIKR), wherein the light chain sequence comprises X2 and X2 comprises the GM-CSF. In some embodiments, the heavy chain sequence comprises a sequence at least about 91%, 92%, 93%, 94%, 95%, 96%, 97%, 98%, 99%, or 100% identical to SEQ ID NO: 2. In some embodiments, the light chain comprises a sequence at least about 90%, 91%, 92%, 93%, 94%, 95%, 96%, 97%, 98%, 99%, or 100% identical to SEQ ID NO: 7. In some embodiments, the light chain comprises a sequence at least about 90%, 91%, 92%, 93%, 94%, 95%, 96%, 97%, 98%, 99%, or 100% identical to SEQ ID NO: 5. In some embodiments, the heavy chain comprises a sequence at least about 90%, 91%, 92%, 93%, 94%, 95%, 96%, 97%, 98%, 99%, or 100% identical to SEQ ID NO: 4. In some embodiments, the heavy chain comprises a sequence at least about 90%, 91%, 92%, 93%, 94%, 95%, 96%, 97%, 98%, 99%, or 100% identical to SEQ ID NO: 1. In some embodiments, the composition further comprises a Fc region comprising reduced effector function as compared to human IgG1. In some embodiments, the human IgG1 comprises SEQ ID NO: 25. In some embodiments, the reduced effector function comprises reduced antibody-dependent cellular cytotoxicity (ADCC). In some embodiments, the reduced effector function comprises reduced complement dependent cytotoxicity (CDC). In some embodiments, the heavy chain further comprises a sequence at least about 90%, 91%, 92%, 93%, 94%, 95%, 96%, 97%, 98%, 99%, or 100% identical to SEQ ID NO: 3; and/or the heavy chain comprises a Fc region comprising a human IgG1 comprising E233P, L234V, L235A, ΔG236, A327G, A330S, P331S, per Kabat numbering.

In one aspect, provided herein is a composition comprising a sequence at least about 90% identical to SEQ ID NO: 18. In some embodiments, the sequence is at least about 91%, 92%, 93%, 94%, 95%, 96%, 97%, 98%, 99%, or 100% identical to SEQ ID NO: 18. In some embodiments, the sequence is connected to an antibody domain. In some embodiments, the antibody domain is an antibody variable domain. In some embodiments, the sequence is positioned within the antibody domain. In some embodiments, the sequence is positioned within a CDR of the antibody variable domain. In some embodiments, the sequence is positioned within the CDR of a modified trastuzumab antibody variable domain. In some embodiments, the sequence is positioned within the CDR of a modified palivizumab antibody variable domain. In some embodiments, the composition comprises a region at least about 90%, 91%, 92%, 93%, 94%, 95%, 96%, 97%, 98%, 99%, or 100% identical to SEQ ID NO: 42, wherein the region comprises the X5, and the X5 comprises the sequence. In some embodiments, the composition further comprises a region at least about 90%, 91%, 92%, 93%, 94%, 95%, 96%, 97%, 98%, 99%, or 100% identical to SEQ ID NO: 31. In some embodiments, the composition comprises a region at least about 90%, 91%, 92%, 93%, 94%, 95%, 96%, 97%, 98%, 99%, or 100% identical to SEQ ID NO: 43, wherein the region comprises the X6, and the X6 comprises the sequence. In some embodiments, the composition further comprises a region at least about 90%, 91%, 92%, 93%, 94%, 95%, 96%, 97%, 98%, 99%, or 100% identical to SEQ ID NO: 2. In some embodiments, the composition further comprises a Fc region comprising reduced effector function as compared to human IgG1. In some embodiments, the human IgG1 comprises SEQ ID NO: 25. In some embodiments, the reduced effector function comprises reduced antibody-dependent cellular cytotoxicity (ADCC). In some embodiments, the reduced effector function comprises reduced complement dependent cytotoxicity (CDC). In some embodiments, the Fc region comprises a sequence at least about 90%, 91%, 92%, 93%, 94%, 95%, 96%, 97%, 98%, 99%, or 100% identical to SEQ ID NO: 3; and/or the Fc region comprises a human IgG1 comprising E233P, L234V, L235A, ΔG236, A327G, A330S, P331S, per Kabat numbering.

In one aspect, provided herein is a composition comprising a first polypeptide comprising SEQ ID NOS: 22, 23, and 16, and a second polypeptide comprising SEQ ID NOS: 19-21. In one aspect, provided herein is a composition comprising a first polypeptide comprising SEQ ID NOS: 22, 23, and 77, and a second polypeptide comprising SEQ ID NOS: 19-21. In some embodiments, the first polypeptide is a light chain of an antibody variable domain. In some embodiments, the second polypeptide is a heavy chain of an antibody variable domain. In some embodiments, the first polypeptide comprises SEQ ID NO: 24. In some embodiments, the composition further comprises a Fc region comprising reduced effector function as compared to human IgG1. In some embodiments, the human IgG1 comprises SEQ ID NO: 25. In some embodiments, the reduced effector function comprises reduced antibody-dependent cellular cytotoxicity (ADCC). In some embodiments, the reduced effector function comprises reduced complement dependent cytotoxicity (CDC). In some embodiments, the second polypeptide further comprises a sequence at least about 90%, 91%, 92%, 93%, 94%, 95%, 96%, 97%, 98%, 99%, or 100% identical to SEQ ID NO: 3; and/or the second polypeptide comprises a Fc region comprising a human IgG1 comprising E233P, L234V, L235A, ΔG236, A327G, A330S, P331S, per Kabat numbering.

In one aspect, provided herein is a composition comprising a first polypeptide comprising SEQ ID NOS: 37-39, and a second polypeptide comprising SEQ ID NOS: 34, 35, 16. In one aspect, provided herein is a composition comprising a first polypeptide comprising SEQ ID NOS: 37-39, and a second polypeptide comprising SEQ ID NOS: 34, 35, 77. In some embodiments, the first polypeptide is a light chain of an antibody variable domain. In some embodiments, the second polypeptide is a heavy chain of an antibody variable domain. In some embodiments, the first polypeptide comprises SEQ ID NO: 36. In some embodiments, the composition further comprises a Fc region comprising reduced effector function as compared to human IgG1. In some embodiments, the human IgG1 comprises SEQ ID NO: 25. In some embodiments, the reduced effector function comprises reduced antibody-dependent cellular cytotoxicity (ADCC). In some embodiments, the reduced effector function comprises reduced complement dependent cytotoxicity (CDC). In some embodiments, the second polypeptide further comprises a sequence at least about 90%, 91%, 92%, 93%, 94%, 95%, 96%, 97%, 98%, 99%, or 100% identical to SEQ ID NO: 3; and/or the second polypeptide comprises a Fc region comprising a human IgG1 comprising E233P, L234V, L235A, ΔG236, A327G, A330S, P331S, per Kabat numbering.

In one aspect, provided herein is a composition comprising a first polypeptide comprising SEQ ID NO: 31, and a second polypeptide comprising a granulocyte macrophage colony stimulating factor (GM-CSF). In some embodiments, the GM-CSF comprises a sequence at least about 85%, 86%, 87%, 88%, 89%, 90%, 91%, 92%, 93%, 94%, 95%, 96%, 97%, 98%, or 99% identical to SEQ ID NO: 16. In some embodiments, the GM-CSF comprises a sequence at least about 85%, 86%, 87%, 88%, 89%, 90%, 91%, 92%, 93%, 94%, 95%, 96%, 97%, 98%, or 99% identical to SEQ ID NO: 77. In some embodiments, the GM-CSF comprises human GM-CSF or murine GM-CSF. In some embodiments, the second polypeptide comprises a modified heavy chain of an antibody variable region. In some embodiments, the modified heavy chain of the antibody variable domain comprises the GM-CSF positioned between a first amino acid sequence of the antibody variable region and a second amino acid sequence of the antibody variable region. In some embodiments, the first amino acid sequence comprises a sequence at least about 90%, 91%, 92%, 93%, 94%, 95%, 96%, 97%, 98%, or 99% identical to SEQ ID NO: 32. In some embodiments, the first amino acid sequence comprises SEQ ID NO: 32. In some embodiments, the second amino acid sequence comprises a sequence at least about 90%, 91%, 92%, 93%, 94%, 95%, 96%, 97%, 98%, or 99% identical to SEQ ID NO: 33. In some embodiments, the second amino acid sequence comprises SEQ ID NO: 33. In some embodiments, the GM-CSF is positioned within a complementarity determining region (CDR) of the modified heavy chain. In some embodiments, the GM-CSF is position within heavy chain CDR1, CDR2, or CDR3. In some embodiments, the GM-CSF is positioned within heavy chain CDR3. In some embodiments, the modified heavy chain is modified from a variable heavy chain comprising SEQ ID NO: 44. In some embodiments, the second polypeptide further comprises a first linker peptide. In some embodiments, the first linker peptide comprises SEQ ID NO: 10. In some embodiments, the first linker peptide comprises SEQ ID NO: 8. In some embodiments, the first linker peptide comprises SEQ ID NO: 11. In some embodiments, the first linker peptide comprises a sequence at least about 90%, 91%, 92%, 93%, 94%, 95%, 96%, 97%, 98%, 99% or 100% identical to SEQ ID NO: 12. In some embodiments, the second polypeptide further comprises a second linker peptide. In some embodiments, the second linker peptide comprises SEQ ID NO: 10. In some embodiments, the second linker peptide comprises SEQ ID NO: 9. In some embodiments, the second linker peptide comprises SEQ ID NO: 11. In some embodiments, the second linker peptide comprises a sequence at least about 90%, 91%, 92%, 93%, 94%, 95%, 96%, 97%, 98%, 99%, or 100% identical to SEQ ID NO: 13. In some embodiments, the second polypeptide comprises a sequence at least about 90%, 91%, 92%, 93%, 94%, 95%, 96%, 97%, 98%, 99%, or 100% identical to SEQ ID NO: 18. In some embodiments, the second polypeptide comprises a sequence at least about 90%, 91%, 92%, 93%, 94%, 95%, 96%, 97%, 98%, 99%, or 100% identical to SEQ ID NO: 29. In some embodiments, the second polypeptide comprises a sequence at least about 90%, 91%, 92%, 93%, 94%, 95%, 96%, 97%, 98%, 99%, or 100% identical to SEQ ID NO: 4. In some embodiments, the second polypeptide comprises a sequence at least about 90%, 91%, 92%, 93%, 94%, 95%, 96%, 97%, 98%, 99%, or 100% identical to SEQ ID NO: 28. In some embodiments, the first polypeptide comprises a light chain of an antibody variable region. In some embodiments, the first polypeptide comprises SEQ ID NO: 31. In some embodiments, the first polypeptide further comprises a sequence at least about 90%, 91%, 92%, 93%, 94%, 95%, 96%, 97%, 98%, 99%, or 100% identical to SEQ ID NO: 7. In some embodiments, the second polypeptide comprises a sequence at least about 90%, 91%, 92%, 93%, 94%, 95%, 96%, 97%, 98%, 99%, or 100% identical to SEQ ID NO: 30. In some embodiments, the first polypeptide and the second polypeptide are connected via one or more disulfide bonds. In some embodiments, the first polypeptide and the second polypeptide form an antibody variable domain. In some embodiments, the antibody variable domain does not bind to an antigen with an equilibrium dissociation constant (KD) lower than about 10-2 M, 10-3 M, or 10-4 M. In some embodiments, the antibody variable domain comprises a modified trastuzumab variable domain. In some embodiments, the modified trastuzumab variable domain comprises a heavy chain CDR1 comprising SEQ ID NO: 34. In some embodiments, the modified trastuzumab variable domain comprises a heavy chain CDR2 comprising SEQ ID NO: 35. In some embodiments, the modified trastuzumab variable domain comprises a heavy chain CDR3 comprising SEQ ID NO: 36, 77 or 16. In some embodiments, the modified trastuzumab variable domain comprises a light chain CDR1 comprising SEQ ID NO: 37. In some embodiments, the modified trastuzumab variable domain comprises a light chain CDR2 comprising SEQ ID NO: 38. In some embodiments, the modified trastuzumab variable domain comprises a light chain CDR3 comprising SEQ ID NO: 39. In some embodiments, the modified trastuzumab variable domain does not bind to human epidermal growth factor receptor 2 (Her2) with a KD lower than about 10-2 M, 10-3 M, or 10-4 M. In some embodiments, the composition further comprises a Fc region comprising reduced effector function as compared to human IgG1. In some embodiments, the human IgG1 comprises SEQ ID NO: 25. In some embodiments, the reduced effector function comprises reduced antibody-dependent cellular cytotoxicity (ADCC). In some embodiments, the reduced effector function comprises reduced complement dependent cytotoxicity (CDC). In some embodiments, the second polypeptide further comprises a sequence at least about 90%, 91%, 92%, 93%, 94%, 95%, 96%, 97%, 98%, 99%, or 100% identical to SEQ ID NO: 3; and/or the second polypeptide comprises a Fc region comprising a human IgG1 comprising E233P, L234V, L235A, ΔG236, A327G, A330S, P331S, per Kabat numbering.

In another aspect, provided herein is a composition comprising an antibody variable domain comprising a light chain sequence comprising a first polypeptide comprising a sequence at least about 90% identical to SEQ ID NO: 31, and a heavy chain sequence comprising a second polypeptide comprising a sequence at least about 90% identical to SEQ ID NO: 29. In some embodiments, the first polypeptide comprises a sequence at least about 91%, 92%, 93%, 94%, 95%, 96%, 97%, 98%, 99%, or 100% identical to SEQ ID NO: 31. In some embodiments, the second polypeptide comprises a sequence at least about 91%, 92%, 93%, 94%, 95%, 96%, 97%, 98%, 99%, or 100% identical to SEQ ID NO: 29. In some embodiments, the composition comprises GM-CSF. In some embodiments, the GM-CSF is human GM-CSF or murine GM-CSF. In some embodiments, GM-CSF comprises a sequence at least about 85%, 86%, 87%, 88%, 89%, 90%, 91%, 92%, 93%, 94%, 95%, 96%, 97%, 98%, or 99% identical to SEQ ID NO: 16. In some embodiments, GM-CSF comprises a sequence at least about 85%, 86%, 87%, 88%, 89%, 90%, 91%, 92%, 93%, 94%, 95%, 96%, 97%, 98%, or 99% identical to SEQ ID NO: 77. In some embodiments, the light chain comprises a sequence at least about 90%, 91%, 92%, 93%, 94%, 95%, 96%, 97%, 98%, 99%, or 100% identical to SEQ ID NO: 7. In some embodiments, the light chain comprises a sequence at least about 90%, 91%, 92%, 93%, 94%, 95%, 96%, 97%, 98%, 99%, or 100% identical to SEQ ID NO: 30. In some embodiments, the heavy chain comprises a sequence at least about 90%, 91%, 92%, 93%, 94%, 95%, 96%, 97%, 98%, 99%, or 100% identical to SEQ ID NO: 4. In some embodiments, the heavy chain comprises a sequence at least about 90%, 91%, 92%, 93%, 94%, 95%, 96%, 97%, 98%, 99%, or 100% identical to SEQ ID NO: 28. In some embodiments, the composition further comprises a Fc region comprising reduced effector function as compared to human IgG1. In some embodiments, the human IgG1 comprises SEQ ID NO: 25. In some embodiments, the reduced effector function comprises reduced antibody-dependent cellular cytotoxicity (ADCC). In some embodiments, the reduced effector function comprises reduced complement dependent cytotoxicity (CDC). In some embodiments, the heavy chain further comprises a sequence at least about 90%, 91%, 92%, 93%, 94%, 95%, 96%, 97%, 98%, 99%, or 100% identical to SEQ ID NO: 3; and/or the heavy chain comprises a Fc region comprising a human IgG1 comprising E233P, L234V, L235A, ΔG236, A327G, A330S, P331S, per Kabat numbering.

In one aspect, provided herein is a composition comprising an antibody variable domain comprising a heavy chain sequence comprising a sequence at least about 90% identical to SEQ ID NO: 42 (EVQLVESGGGLVQPGGSLRLSCAASGFNIKDTYIHWVRQAPGKGLEWVARITYPTNGYTRYADSVKG RFTISADTSKNTAYLQMNSLRAEDTAVYYCSRWGQGTLVTVSS), wherein the heavy chain sequence comprises X6 and X6 comprises GM-CSF; and a light chain sequence comprising a sequence at least about 90% identical to SEQ ID NO: 31. In some embodiments, the GM-CSF is human GM-CSF or murine GM-CSF. In some embodiments, GM-CSF comprises a sequence at least about 85%, 86%, 87%, 88%, 89%, 90%, 91%, 92%, 93%, 94%, 95%, 96%, 97%, 98%, or 99% identical to SEQ ID NO: 16. In some embodiments, GM-CSF comprises a sequence at least about 85%, 86%, 87%, 88%, 89%, 90%, 91%, 92%, 93%, 94%, 95%, 96%, 97%, 98%, or 99% identical to SEQ ID NO: 77. In some embodiments, the heavy chain sequence comprises a sequence at least about 91%, 92%, 93%, 94%, 95%, 96%, 97%, 98%, 99%, or 100% identical to SEQ ID NO: 42. In some embodiments, the heavy chain sequence comprises a sequence at least about 90%, 91%, 92%, 93%, 94%, 95%, 96%, 97%, 98%, 99%, or 100% identical to SEQ ID NO: 43 (EVQLVESGGGLVQPGGSLRLSCAASGFNIKDTYIHWVRQAPGKGLEWVARITYPTNGYTRYADSVKG RFTISADTSKNTAYLQMNSLRAEDTAVYYCSRGGSGAKLAALKAKLAALKGGGGS[[X6]]GGGGSEL AALEAELAALEAGGSGDYWGQGTLVTVSS), wherein the heavy chain sequence comprises X6 and X6 comprises the GM-CSF. In some embodiments, the heavy chain sequence comprises a sequence at least about 91%, 92%, 93%, 94%, 95%, 96%, 97%, 98%, 99%, or 100% identical to SEQ ID NO: 43. In some embodiments, the light chain comprises a sequence at least about 90%, 91%, 92%, 93%, 94%, 95%, 96%, 97%, 98%, 99%, or 100% identical to SEQ ID NO: 7. In some embodiments, the heavy chain comprises a sequence at least about 90%, 91%, 92%, 93%, 94%, 95%, 96%, 97%, 98%, 99%, or 100% identical to SEQ ID NO: 29. In some embodiments, the heavy chain comprises a sequence at least about 90%, 91%, 92%, 93%, 94%, 95%, 96%, 97%, 98%, 99%, or 100% identical to SEQ ID NO: 4. In some embodiments, the heavy chain comprises a sequence at least about 90%, 91%, 92%, 93%, 94%, 95%, 96%, 97%, 98%, 99%, or 100% identical to SEQ ID NO: 28. In some embodiments, the composition further comprises a Fc region comprising reduced effector function as compared to human IgG1. In some embodiments, the human IgG1 comprises SEQ ID NO: 25. In some embodiments, the reduced effector function comprises reduced antibody-dependent cellular cytotoxicity (ADCC). In some embodiments, the reduced effector function comprises reduced complement dependent cytotoxicity (CDC). In some embodiments, the heavy chain further comprises a sequence at least about 90%, 91%, 92%, 93%, 94%, 95%, 96%, 97%, 98%, 99%, or 100% identical to SEQ ID NO: 3; and/or the heavy chain comprises a Fc region comprising a human IgG1 comprising E233P, L234V, L235A, ΔG236, A327G, A330S, P331S, per Kabat numbering.

In one aspect, provided herein are methods of use, wherein any of the compositions herein are used for the treatment of a neurological disease or condition. Also provided herein are methods of treating a neurological disease or condition, comprising administering to a subject in need thereof any of the compositions described herein. In some embodiments, the neurological disease or condition comprises Parkinson's disease. Also provided herein are methods of treating Alzheimer's disease, comprising administering to a subject in need thereof any of the compositions described herein. Also provided herein are methods of treating traumatic brain injury, comprising administering to a subject in need thereof any of the compositions described herein. Also provided herein are methods of treating amyotrophic lateral sclerosis (ALS), comprising administering to a subject in need thereof any of the compositions described herein. Also provided herein are methods of treating acute radiation syndrome, comprising administering to a subject in need thereof any of the compositions described herein. Also provided herein are methods of treating cancer, comprising administering to a subject in need thereof any of the compositions described herein. In some embodiments, the composition is administered once every about 7, 8, 9, 10, 11, 12, 13, 14, 15, 16, 17, 18, 19, 20, 21, 22, 23, 24, 25, 26, 27, 28, 29, or 30 days during a treatment period. In some embodiments, the composition is administered once every about 14 days during a treatment period. In some embodiments, the composition is administered once every about 2 weeks during a treatment period. In some embodiments, the composition is administered once every about 3 weeks during a treatment period. In some embodiments, the composition is administered once every about 4 weeks during a treatment period. In some embodiments, the composition is administered about once a month during a treatment period. In some embodiments, the treatment period comprises from about 8 weeks to about 2 years.

### BRIEF DESCRIPTION OF THE DRAWINGS

The foregoing summary, as well as the following detailed description of the disclosure, will be better understood when read in conjunction with the appended figures. However, the disclosure is not limited to the precise examples shown and according to common practice, the various features of the drawings are not to-scale. In some cases, the dimensions of the various features are arbitrarily expanded or reduced for clarity. Included in the drawings are the following figures.

FIGS. 1A-L: Her-mGMCSF CDR treatment affects T cell populations in peripheral blood and spleen. Quantification of CD8+ levels (FIG. 1A), CD4+ levels (FIG. 1B), and CD4+CD25+FoxP3+ regulatory T cell (T_{reg}) levels (FIG. 1C) in peripheral blood of mice treated with ascending doses of Her-mGMCSF CDR. Differences in means (±SEM, n=5) were determined where p<0.05 compared with (a) 0 mg/kg, (b) 0.3 mg/kg, (c) 1.0 mg/kg, (d) 3.0 mg/kg, or (e) 10.0 mg/kg treatment. Quantification of CD8+ levels (FIG. 1D), CD4+ levels (FIG. 1E), and CD4+CD25+FoxP3+T_{reg} levels (FIG. 1F) in peripheral blood of mice treated with ascending doses of rGM-CSF. Quantification of CD8+ levels (FIG. 1G), CD4+ levels (FIG. 1H), and CD4+CD25+FoxP3+T_{reg} levels (FIG. 1I) in spleens isolated from mice treated with ascending doses of Her-mGMCSF CDR. Differences in means (±SEM, n=5) were determined where p<0.05 compared with (a) 0 mg/kg treatment, (b) 0.3 mg/kg treatment, (c) 1.0 mg/kg treatment, and (d) 3.0 mg/kg treatment. Quantification of CD8+ levels (FIG. 1J), CD4+ levels (FIG. 1K), and CD4+CD25+FoxP3+T_{reg} levels (FIG. 1L) in spleens isolated from mice treated ascending doses of rGM-CSF. Differences in means (±SEM, n=5) were determined where p<0.05 compared with (a) 0 mg/kg, (b) 0.01 mg/kg, (c) 0.03 mg/kg, or (d) 0.10 mg/kg rGM-CSF treatment. Treatment with both Her-mGMCSF CDR and rGM-CSF resulted in a significant dose-dependent increase in T_{reg} levels within the spleen. Linear regression analysis results are provided on graphs (FIG. 1I) and (FIG. 1L).

FIG. 2: Her-mGMCSF CDR treatment decreases the neuroinflammatory response observed following MPTP intoxication. Quantification of reactive microglia within the substantia nigra two days post MPTP-intoxication. Differences in means (±SEM, n=5) were determined where p<0.05 compared with (a) PBS or (b) MPTP treatment.

FIG. 3: Her-mGMCSF CDR treatment spares dopaminergic neurons following MPTP intoxication. Stereological quantification of total number of surviving dopaminergic (TH+/Niss1+) and non-dopaminergic (TH-/Niss1+) neurons within the substantia nigra following MPTP intoxication. Differences in means (±SEM, n=7) were determined where p<0.05 compared with groups treated with (a) PBS or (b) MPTP. Mean percent remaining total neuron number is indicated on each treatment bar.

FIG. 4: Her-mGMCSF CDR treatment attenuates striatal termini loss. Densitometry analysis of TH+ termini within the striatum following MPTP intoxication. Treatment groups are normalized to PBS control density. Differences in means (±SEM, n=7) were determined where p<0.05 compared with groups treated with (a) PBS or (b) MPTP.

FIGS. 5A-B: Her-mGMCSF CDR treatment exhibits long-acting anti-inflammatory and immune-modulating properties. FIG. 5A: Quantification of reactive microglia (mac-1+) within the substantia nigra two days post MPTP-intoxication. Differences in means (±SEM, n=5) were determined where p<0.05 compared with (a) PBS or (b) MPTP treatment. FIG. 5B: Stereological quantification of total number of surviving dopaminergic (TH+/Niss1+) and non-dopaminergic (TH-/Niss1+) neurons within the substantia nigra seven days post MPTP intoxication. Differences in means (±SEM, n=5) were determined where p<0.05 compared with groups treated with (a) PBS, (b) MPTP, (c) Day −15 Her-mGMCSF CDR +MPTP, and (d) Day −10 Her-mGMCSF CDR+MPTP.

FIGS. 6A-B: Potency of long-acting GM-CSF molecules Syn hGMCSF CDRL3, NhGM Syn HC and NhGM Syn LC (FIG. 6A) and Her hGMCSF CDR (FIG. 6B), in TF-1 proliferation assays.

FIG. 7: Schematics of the Fab domains of the various long-acting GM-CSF molecules, where GM-CSF is positioned at an amino-terminus or CDR of an IgG scaffold.

FIGS. 8A-B: GM-CSF molecules Syn-hGMCSF CDR, Her-hGMCSF CDR, Syn-mGMCSF CDR, and Syn-mGMCSF NT (HC fusion) exhibit increased half-life as compared to recombinant GM-CSF in rat and mouse plasma, respectively. FIG. 8A shows the concentrations of Syn-hGMCSF CDR and Her-hGMCSF CDR in rat plasma over time. FIG. 8B shows the concentrations of Syn-mGMCSF CDR and Syn-mGMCSF NT (N-terminal HC fusion) in mouse plasma over time.

FIG. 9: Sub-chronic treatment with Syn mGMCSF CDR significantly increases T_{reg} expansion in mice.

FIG. 10: Long-acting GM-CSF, Her-hGMCSF CDR, increased T_{reg} expansion for up to 14 days.

FIG. 11: Pharmacokinetic and pharmacodynamic studies in cynomolgus. Her-hGMCSF CDR dose-dependently increases circulating T_{reg}.

FIG. 12: Long-acting GM-CSF is actively transported into the brain of mice.

### DETAILED DESCRIPTION OF THE INVENTION

Described herein are GM-CSF molecules and compositions comprising GM-CSF molecules. Exemplary molecules comprise a GM-CSF peptide connected to a scaffold that increases the half-life of the GM-CSF peptide to greater than the half-life of GM-CSF alone. Such molecules may be referred to as long-acting GM-CSF molecules. Exemplary scaffolds for increasing GM-CSF half-life include antibody variable domains, where the GM-CSF is connected optionally via one or more linkers to the antibody variable domain. In some cases, the antibody variable domain has reduced or no antigen binding. A reduction of antigen binding may be generated by modifying a complementary determining region (CDR) of the antibody variable domain. The modification may include insertion of the GMCSF peptide and/or mutation, addition, or deletion of one or more CDR amino acids. Antibody scaffolds may also comprise a fragment crystallizable (Fc) region that exhibits reduced effector function, such as reduced antibody-dependent cytotoxicity (ADCC) and/or reduced complement dependent cytotoxicity (CDC), as compared to an antibody scaffold comprising a wild-type IgG1 Fc region.

Various GM-CSF molecules described herein increase T_{reg} number and/or function that could be beneficial for Parkinson's disease (PD) and/or other neurodegenerative and neuroinflammatory diseases. For instance, as shown in the examples herein, treatment with long-acting GM-CSF resulted in a dose-dependent increase in T_{reg} numbers after a single injection that resulted in increased cellular function within the peripheral blood and spleen above those observed with recombinant GM-CSF (rGM-CSF) alone. In addition, T_{reg} isolated from mice treated with long-acting GM-CSF displayed increased anti-proliferative effects and were able to suppress Tresp proliferation to a greater extent than T_{reg} isolated from rGM-CSF treated mice. Clinically, modifying diseased T_{reg} populations has been tested in both ALS and PD. ALS patients display dysfunctional T_{reg} that correlate with disease severity and survival. However, if diseased T_{reg} are isolated and stimulated ex vivo, suppressive function is restored, suggesting a potential therapeutic target. Various GM-CSF molecules described herein also exhibit neuroprotective properties. As shown in the examples, a single dose of long-acting GM-CSF was neuroprotective in a MPTP mouse model.

Before the present methods and compositions are described, it is to be understood that this disclosure is not limited to a particular method or composition described. The terminology used is for the purpose of describing particular embodiments only, and is not intended to be limiting, since the scope of the present disclosure will be limited only by the appended claims. Examples are put forth so as to provide those of ordinary skill in the art with a disclosure and description of how to make and use the present compositions and methods, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

As used herein and in the appended claims, the singular forms “a”, “an”, and “the” include plural referents unless the context clearly dictates otherwise. For example, reference to “a cell” includes a plurality of such cells and reference to “the peptide” includes reference to one or more peptides and equivalents thereof, e.g. polypeptides, known to those skilled in the art, and so forth.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDRH1, CDRH2, CDRH3) and three CDRs in each light chain variable region (CDRL1, CDRL2, CDRL3). In some embodiments, an antibody scaffold in a GM-CSF molecule provided herein comprises one or more amino acid mutations, additions, and/or deletions to one or more CDRs such that the CDRs have reduced or no antigen-binding Such modified antibody scaffolds may still be considered to comprise six CDRs (CDRH1, CDRH2, CDRH3, CDRL1, CDRL2, CDRL3) without requiring antigen-binding, where the CDRs are positioned between framework regions of the antibody (e.g., heavy chain comprises FRH1-CDRH1-FRH2-CDRH2-FRH3-CDRH3-FRH4 and light chain comprises FRL1-CDRL1-FRL2-CDRL2-FRL3-CDRL3-FRL4). In some embodiments, a CDR comprises GM-CSF, where the GM-CSF has replaced one or more amino acids of the CDR. “Framework regions” and “FR” are known in the art to refer to the non-CDR portions of the variable regions of the heavy and light chains. In general, there are four FRs in each full-length heavy chain variable region (FRH1, FRH2, FRH3, and FRH4), and four FRs in each full-length light chain variable region (FRL1, FRL2, FRL3, and FRL4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc MP et al.,“IMGT unique numbering for immunoglobulin and T cell receptor variable domains and Ig superfamily V-like domains,” Dev Comp Immunol, 2003 Jan;27(1):55-77 (“IMGT” numbering scheme); Honegger A and Plückthun A, “Yet another numbering scheme for immunoglobulin variable domains: an automatic modeling and analysis tool,” J Mol Biol, 2001 Jun 8;309(3):657-70, (“Aho” numbering scheme); and Whitelegg NR and Rees AR, “WAM: an improved algorithm for modelling antibodies on the WEB,” Protein Eng. 2000 Dec;13(12):819-24 (“AbM” numbering scheme. In certain embodiments, the CDRs of the antibodies described herein can be defined by a method selected from Kabat, Chothia, IMGT, Aho, AbM, or combinations thereof.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program BLAST by NCBI.

### GM-CSF Peptides

In one aspect, provided herein are GM-CSF molecules comprising GM-CSF peptides, such as human, bovine, rat and/or mouse GM-CSF. In some embodiments, a GM-CSF peptide comprises an amino acid sequence that is identical to or at least about 80%, 85%, 90%, 91%, 92%, 93%, 94%, 95%, 96%, 97%, 98%, 99%, or 100% identical to SEQ ID NO: 16. Long-acting GM-CSF molecules provided herein may comprise a GM-CSF peptide comprising an amino acid sequence that is identical to or at least about 80%, 85%, 90%, 91%, 92%, 93%, 94%, 95%, 96%, 97%, 98%, 99%, or 100% identical to SEQ ID NO: 16. Long-acting GM-CSF molecules provided herein may comprise a GM-CSF peptide variant comprising an amino acid sequence having about 1, 2, 3, 4, 5, 6, 7, 8, 9, or 10 amino acid additions, deletions, or substitutions as compared to GM-CSF comprising SEQ ID NO: 16. In some embodiments, a GM-CSF peptide comprises an amino acid sequence that is identical to or at least about 80%, 85%, 90%, 91%, 92%, 93%, 94%, 95%, 96%, 97%, 98%, 99%, or 100% identical to SEQ ID NO: 77. Long-acting GM-CSF molecules provided herein may comprise a GM-CSF peptide comprising an amino acid sequence that is identical to or at least about 80%, 85%, 90%, 91%, 92%, 93%, 94%, 95%, 96%, 97%, 98%, 99%, or 100% identical to SEQ ID NO: 77. Long-acting GM-CSF molecules provided herein may comprise a GM-CSF peptide variant comprising an amino acid sequence having about 1, 2, 3, 4, 5, 6, 7, 8, 9, or 10 amino acid additions, deletions, or substitutions as compared to GM-CSF comprising SEQ ID NO: 77. Such GM-CSF peptide variants include those having one or more conservative amino acid substitutions. A conservative substitution may involve a substitution found in one of the following conservative substitutions groups: Group 1: Alanine (Ala; A), Glycine (Gly; G), Serine (Ser; S), Threonine (Thr; T); Group 2: Aspartic acid (Asp; D), Glutamic acid (Glu; E); Group 3: Asparagine (Asn; N), Glutamine (Gln; Q); Group 4: Arginine (Arg; R), Lysine (Lys; K), Histidine (His; H); Group 5: Isoleucine (Ile; I), Leucine (Leu; L), Methionine (Met; M), Valine (Val; V); and Group 6: Phenylalanine (Phe; F), Tyrosine (Tyr; Y), Tryptophan (Trp; W). Additionally, amino acids may be grouped into conservative substitution groups by similar function, chemical structure, or composition. For example, an aliphatic grouping may include, for purposes of substitution, Gly, Ala, Val, Leu, and Ile. Other groups including amino acids that are considered conservative substitutions for one another may include: sulfur-containing: Met and Cys; acidic: Asp, Glu, and Asn; small aliphatic, nonpolar or slightly polar residues: Ala, Ser, Thr, Pro, and Gly; polar, negatively charged residues and their amides: Asp, Asn, and Glu; polar, positively charged residues: His, Arg, and Lys; large aliphatic, nonpolar residues: Met, Leu, Ile, Val, and Cys; and large aromatic residues: Phe, Tyr, and Trp.

Long-Acting GM-CSF Molecules

In one aspect, provided herein are GM-CSF molecules connected to a scaffold to increase the half-life of a GM-CSF peptide, sometimes referred to as long-acting GM-CSF molecules. A non-limiting example of a scaffold includes an antibody variable domain. The scaffold may comprise the variable regions of a heavy (VH) and/or light chain (VL), and/or one or more constant regions of a full-length antibody. Therefore, as used herein, a scaffold having an antibody variable domain is inclusive of Fab, full-length antibody, and any other antibodies comprising an antibody variable domain. The GM-CSF peptide need not be connected directly to the antibody, and may be connected via one or more linker molecules. In some cases, GM-CSF is positioned at a terminus of the antibody heavy chain or light chain. In some cases, GM-CSF is positioned within and/or replaces one or more amino acids of a CDR of the antibody variable domain. In some cases, the GM-CSF is positioned between two amino acids of the CDR, the GM-CSF is positioned between the antibody and the first amino acid of the CDR, the GM-CSF is positioned between the antibody and the last amino acid of the CDR, and/or the GM-CSF replaces a portion or all of the CDR and thus is positioned where the CDR previously existed. In some cases, a GM-CSF molecule comprises a first antibody portion, a GM-CSF peptide, and a second antibody portion. For example, the first antibody portion comprises one or more framework regions and, if applicable, any other CDRs N-terminal to the CDR where the GM-CSF is positioned, and the second antibody portion comprises one or more framework regions and/or Fc regions, and if applicable, any other CDRs C-terminal the CDR where the GM-CSF peptide is positioned. Each of the first and second antibody portions may independently have a length selected from: at least about 10, at least about 15, at least about 20, at least about 25, at least about 30, at least about 35, at least about 40, at least about 45, or at least about 50 amino acids. For a palivizumab scaffold, the first antibody portion may comprise a sequence at least about 95%, 96%, 97%, 98%, 99%, or 100% identical to SEQ ID NO: 14. For a palivizumab scaffold, the second antibody portion may comprise a sequence at least about 95%, 96%, 97%, 98%, 99%, or 100% identical to SEQ ID NO: 15. For a trastuzumab scaffold, the first antibody portion may comprise a sequence at least about 95%, 96%, 97%, 98%, 99%, or 100% identical to SEQ ID NO: 32. For a trastuzumab scaffold, the second antibody portion may comprise a sequence at least about 95%, 96%, 97%, 98%, 99%, or 100% identical to SEQ ID NO: 33. In some cases, positioned within the CDR indicates that no amino acids of the CDR are deleted. In some cases, positioned within the CDR indicates that at least 1, 2, 3, 4, 5, 6, 7, 8 or the entire CDR is replaced by the GM-CSF peptide. In some cases, an antibody CDR comprises a GM-CSF peptide sequence. For instance, a heavy or light chain CDR3 comprises a GM-CSF peptide sequence.

In various GM-CSF molecules, the GM-CSF peptide is connected to a scaffold by one or more linkers. In some embodiments, a linker comprises a sequence configured to form an alpha helix. In some embodiments, a linker comprises a sequence configured to have no regular secondary structure (e.g., no alpha helix, 3-10 helix, beta-strand, beta turn). Non-limiting exemplary linkers may comprise one or more of SEQ ID NOS: 8-13.

The connections discussed herein may include peptide bonds, and as such, the GM-CSF molecules may be produced from a genetic construct comprising DNA encoding for GM-CSF fusion molecules. As used herein, “positioned within” and “inserted” may indicate the location of a GM-CSF peptide within a polypeptide comprising both the GM-CSF peptide and scaffold, and as such, may not be not indicative of the method in which the GM-CSF molecule is produced. For instance, “inserted” does not necessarily limit the molecules to those generated by modifying the DNA encoding the scaffold by insertion of the DNA encoding for the GM-CSF peptide, but may also or alternatively indicate that the DNA encoding the scaffold and GM-CSF peptide is de novo synthesized.

In various GM-CSF molecules, the GM-CSF peptide is connected to a scaffold comprising an antibody variable domain. The antibody variable domain may comprise a first polypeptide and a second polypeptide, wherein the first or second polypeptide comprise or are otherwise connected to GM-CSF. In some cases, the first polypeptide comprises a light chain of an antibody variable domain and the second polypeptide comprises a heavy chain variable domain. In some cases, the first polypeptide comprises a heavy chain of an antibody variable domain and the second polypeptide comprises a light chain variable domain. Non-limiting exemplary antibody variable domains include a trastuzumab or palivizumab variable domain, with may be modified with connection to a GM-CSF peptide. The trastuzumab or palivizumab variable domain may comprise a modification that reduces antigen binding as compared to non-modified trastuzumab or palivizumab (e.g., unmodified antibodies Herceptin, Synagis, respectively). In some cases, the GM-CSF peptide is connected to an amino terminus of the light chain or heavy chain. In some cases, the GM-CSF peptide is positioned within the light chain or heavy chain. For instance, the GM-CSF peptide is positioned within the light chain or heavy chain CDR. As a further non-limiting example, the GM-CSF peptide is positioned within the light chain or heavy chain CDR3.

In some embodiments, the scaffold comprises an antibody Fc region comprising reduced effector function as compared to human IgG1 (SEQ ID NO: 25). The reduced effector function may comprise reduced antibody-dependent cellular cytotoxicity (ADCC) and/or reduced complement dependent cytotoxicity (CDC). In some cases, the scaffold comprises a Fc sequence comprising at least about 90%, 91%, 92%, 93%, 94%, 95%, 96%, 97%, 98%, 99%, or 100% identity to SEQ ID NO: 3. In some cases, the scaffold comprises a Fc region comprising a human IgG1 comprising E233P, L234V, L235A, ΔG236, A327G, A330S, P331S, per Kabat numbering.

In some embodiments, the first polypeptide comprises a sequence at least about 90%, 91%, 92%, 93%, 94%, 95%, 96%, 97%, 98%, 99%, or 100% identical to SEQ ID NO: 6. In some cases, the first polypeptide comprises a sequence at least about 90%, 91%, 92%, 93%, 94%, 95%, 96%, 97%, 98%, 99%, or 100% identical to SEQ ID NO: 7. In some cases, the first polypeptide comprises a sequence at least about 90%, 91%, 92%, 93%, 94%, 95%, 96%, 97%, 98%, 99%, or 100% identical to SEQ ID NO: 5. In some embodiments, the second polypeptide comprises a sequence at least about 90%, 91%, 92%, 93%, 94%, 95%, 96%, 97%, 98%, 99%, or 100% identical to SEQ ID NO: 2. In some cases, the second polypeptide comprises a sequence at least about 90%, 91%, 92%, 93%, 94%, 95%, 96%, 97%, 98%, 99%, or 100% identical to SEQ ID NO: 4. In some cases, the second polypeptide comprises a sequence at least about 90%, 91%, 92%, 93%, 94%, 95%, 96%, 97%, 98%, 99%, or 100% identical to SEQ ID NO: 1. In some embodiments, provided is a GM-CSF molecule comprising an antibody variable domain comprising a light chain sequence comprising a first polypeptide comprising a sequence at least about 90%, 91%, 92%, 93%, 94%, 95%, 96%, 97%, 98%, 99%, or 100% identical to SEQ ID NO: 6, and a heavy chain sequence comprising a second polypeptide comprising a sequence at least about 90%, 91%, 92%, 93%, 94%, 95%, 96%, 97%, 98%, 99%, or 100% identical to SEQ ID NO: 2.

In some embodiments, the first polypeptide comprises a sequence at least about 90%, 91%, 92%, 93%, 94%, 95%, 96%, 97%, 98%, 99%, or 100% identical to SEQ ID NO: 31. In some cases, the first polypeptide comprises a sequence at least about 90%, 91%, 92%, 93%, 94%, 95%, 96%, 97%, 98%, 99%, or 100% identical to SEQ ID NO: 30. In some cases, the first polypeptide comprises a sequence at least about 90%, 91%, 92%, 93%, 94%, 95%, 96%, 97%, 98%, 99%, or 100% identical to SEQ ID NO: 7. In some embodiments, the second polypeptide comprises a sequence at least about 90%, 91%, 92%, 93%, 94%, 95%, 96%, 97%, 98%, 99%, or 100% identical to SEQ ID NO: 29. In some cases, the second polypeptide comprises a sequence at least about 90%, 91%, 92%, 93%, 94%, 95%, 96%, 97%, 98%, 99%, or 100% identical to SEQ ID NO: 28. In some cases, the second polypeptide comprises a sequence at least about 90%, 91%, 92%, 93%, 94%, 95%, 96%, 97%, 98%, 99%, or 100% identical to SEQ ID NO: 4. In some embodiments, provided is a GM-CSF molecule comprising an antibody variable domain comprising a light chain sequence comprising a first polypeptide comprising a sequence at least about 90%, 91%, 92%, 93%, 94%, 95%, 96%, 97%, 98%, 99%, or 100% identical to SEQ ID NO: 31, and a heavy chain sequence comprising a second polypeptide comprising a sequence at least about 90%, 91%, 92%, 93%, 94%, 95%, 96%, 97%, 98%, 99%, or 100% identical to SEQ ID NO: 29.

In various GM-CSF molecules, the GM-CSF peptide is connected to a scaffold comprising an antibody variable domain comprising a light chain sequence comprising a sequence at least about 90% identical to SEQ ID NO: 26 (DIQMTQSPSTLSASVGDRVTITCKCQLSVGYMHWYQQKPGKAPKLLIYDTSKLASGVPSRFSGSGSG TEFTLTISSLQPDDFATYYCFQGS[X1]PFTFGGGTKLEIKR), wherein the light chain sequence comprises X1 and X1 comprises GM-CSF; and a heavy chain sequence comprising a sequence at least about 90%, 91%, 92%, 93%, 94%, 95%, 96%, 97%, 98%, 99%, or 100% identical to SEQ ID NO: 2. The GM-CSF may be human, bovine or murine GM-CSF. The GM-CSF may comprise a sequence at least about 85%, 86%, 87%, 88%, 89%, 90%, 91%, 92%, 93%, 94%, 95%, 96%, 97%, 98%, 99%, or 100% identical to SEQ ID NO: 16. The GM-CSF may comprise a sequence at least about 85%, 86%, 87%, 88%, 89%, 90%, 91%, 92%, 93%, 94%, 95%, 96%, 97%, 98%, 99%, or 100% identical to SEQ ID NO: 77. The GM-CSF may include a variant or homolog of GM-CSF. In some embodiments, the light chain sequence comprises a sequence at least about 91%, 92%, 93%, 94%, 95%, 96%, 97%, 98%, 99%, or 100% identical to SEQ ID NO: 26. In some embodiments, the light chain sequence comprises a sequence at least about 90%, 91%, 92%, 93%, 94%, 95%, 96%, 97%, 98%, 99%, or 100% identical to SEQ ID NO: 27 (DIQMTQSPSTLSASVGDRVTITCKCQLSVGYMHWYQQKPGKAPKLLIYDTSKLASGVPSRFSGSGSG TEFTLTISSLQPDDFATYYCFQGSGGSGAKLAALKAKLAALKGGGGS[X2]GGGGSELAALEAELAAL EAGGSGPFTFGGGTKLEIKR), wherein the light chain sequence comprises X2 and X2 comprises the GM-CSF. In some embodiments, the heavy chain sequence comprises a sequence at least about 91%, 92%, 93%, 94%, 95%, 96%, 97%, 98%, 99%, or 100% identical to SEQ ID NO: 2. In some embodiments, the light chain comprises a sequence at least about 90%, 91%, 92%, 93%, 94%, 95%, 96%, 97%, 98%, 99%, or 100% identical to SEQ ID NO: 7. In some embodiments, the light chain comprises a sequence at least about 90%, 91%, 92%, 93%, 94%, 95%, 96%, 97%, 98%, 99%, or 100% identical to SEQ ID NO: 5. In some embodiments, the heavy chain comprises a sequence at least about 90%, 91%, 92%, 93%, 94%, 95%, 96%, 97%, 98%, 99%, or 100% identical to SEQ ID NO: 4. In some embodiments, the heavy chain comprises a sequence at least about 90%, 91%, 92%, 93%, 94%, 95%, 96%, 97%, 98%, 99%, or 100% identical to SEQ ID NO: 1. In some embodiments, the GM-CSF molecule further comprises a Fc region comprising reduced effector function as compared to human IgG1 (SEQ ID NO: 25). The reduced effector function may comprise reduced antibody-dependent cellular cytotoxicity (ADCC) and reduced complement dependent cytotoxicity (CDC). The Fc region may comprise a sequence at least about 90%, 91%, 92%, 93%, 94%, 95%, 96%, 97%, 98%, 99%, or 100% identical to SEQ ID NO: 3. In some cases, the Fc region comprises a human IgG1 comprising E233P, L234V, L235A, ΔG236, A327G, A330S, P331S, per Kabat numbering.

In various GM-CSF molecules, the GM-CSF peptide is connected to a scaffold comprising an antibody variable domain comprising a heavy chain sequence comprising a sequence at least about 90% identical to SEQ ID NO: 42 (EVQLVESGGGLVQPGGSLRLSCAASGFNIKDTYIHWVRQAPGKGLEWVARITYPTNGYTRYADSVKG RFTISADTSKNTAYLQMNSLRAEDTAVYYCSR[[X5]]WGQGTLVTVSS), wherein the heavy chain sequence comprises X6 and X6 comprises the GM-CSF peptide; and a light chain sequence comprising a sequence at least about 90%, 91%, 92%, 93%, 94%, 95%, 96%, 97%, 98%, 99%, or 100% identical to SEQ ID NO: 31. The GM-CSF may be human, bovine or murine GM-CSF. The GM-CSF may comprise a sequence at least about 85%, 86%, 87%, 88%, 89%, 90%, 91%, 92%, 93%, 94%, 95%, 96%, 97%, 98%, 99%, or 100% identical to SEQ ID NO: 16. The GM-CSF may comprise a sequence at least about 85%, 86%, 87%, 88%, 89%, 90%, 91%, 92%, 93%, 94%, 95%, 96%, 97%, 98%, 99%, or 100% identical to SEQ ID NO: 77. The GM-CSF may include a variant or homolog of GM-CSF. In some embodiments, the heavy chain sequence comprises a sequence at least about 91%, 92%, 93%, 94%, 95%, 96%, 97%, 98%, 99%, or 100% identical to SEQ ID NO: 42. In some embodiments, the heavy chain sequence comprises a sequence at least about 90%, 91%, 92%, 93%, 94%, 95%, 96%, 97%, 98%, 99%, or 100% identical to SEQ ID NO: 43 (EVQLVESGGGLVQPGGSLRLSCAASGFNIKDTYIHWVRQAPGKGLEWVARITYPTNGYTRYADSVKG RFTISADTSKNTAYLQMNSLRAEDTAVYYCSRGGSGAKLAALKAKLAALKGGGGS[[X6]]GGGGSEL AALEAELAALEAGGSGDYWGQGTLVTVSS), wherein the heavy chain sequence comprises X6 and X6 comprises the GM-CSF. In some embodiments, the heavy chain sequence comprises a sequence at least about 91%, 92%, 93%, 94%, 95%, 96%, 97%, 98%, 99%, or 100% identical to SEQ ID NO: 43. In some embodiments, the light chain comprises a sequence at least about 90%, 91%, 92%, 93%, 94%, 95%, 96%, 97%, 98%, 99%, or 100% identical to SEQ ID NO: 7. In some embodiments, the heavy chain comprises a sequence at least about 90%, 91%, 92%, 93%, 94%, 95%, 96%, 97%, 98%, 99%, or 100% identical to SEQ ID NO: 29. In some embodiments, the heavy chain comprises a sequence at least about 90%, 91%, 92%, 93%, 94%, 95%, 96%, 97%, 98%, 99%, or 100% identical to SEQ ID NO: 4. In some embodiments, the heavy chain comprises a sequence at least about 90%, 91%, 92%, 93%, 94%, 95%, 96%, 97%, 98%, 99%, or 100% identical to SEQ ID NO: 28. In some embodiments, the GM-CSF molecule further comprises a Fc region comprising reduced effector function as compared to human IgG1 (SEQ ID NO: 25). The reduced effector function may comprise reduced antibody-dependent cellular cytotoxicity (ADCC) and reduced complement dependent cytotoxicity (CDC). The Fc region may comprise a sequence at least about 90%, 91%, 92%, 93%, 94%, 95%, 96%, 97%, 98%, 99%, or 100% identical to SEQ ID NO: 3. In some cases, the Fc region comprises a human IgG1 comprising E233P, L234V, L235A, ΔG236, A327G, A330S, P331S, per Kabat numbering.

In one aspect, provided herein are GM-CSF molecules comprising a first linker, a GM-CSF peptide, and a second linker. In non-limiting examples, the GM-CSF peptide may be human, bovine, rat or mouse. For instance, the GM-CSF may comprise a sequence at least about 90%, 91%, 92%, 93%, 94%, 95%, 96%, 97%, 98%, 99%, or 100% identical to SEQ ID NO: 16. The GM-CSF may comprise a sequence at least about 90%, 91%, 92%, 93%, 94%, 95%, 96%, 97%, 98%, 99%, or 100% identical to SEQ ID NO: 77. In some cases, the GM-CSF peptide comprises a homolog or variant of GM-CSF. In some embodiments, the first linker comprises a first peptide configured to form an alpha helix. Alpha helical formation may be predicted based on analysis of primary structure using available bioinformatic tools readily available in the art. In some embodiments, the second linker comprises a second peptide configured to form an alpha helix. In some embodiments, the GM-CSF molecule comprises the first and second peptides that are configured to form a coiled-coil. The coil-coil may be an anti-parallel coiled-coil. The first peptide may comprise a sequence having no or less than about 2, 3, or 4 amino acid substitutions or deletions from SEQ ID NO: 8. The second peptide may comprise a sequence having no or less than about 2, 3, or 4 amino acid substitutions or deletions from SEQ ID NO: 9. In some embodiments, the first linker comprises an amino acid sequence comprising at least about 2, 3, 4, 5, 6, 7, 8, 9, or 10 amino acids (and up to about 30 amino acids), wherein the amino acid sequence does not comprise a regular secondary structure (e.g., alpha helix, beta strand, 310 helix, beta turn) and/or is a flexible linker. In some embodiments, the second linker comprises an amino acid sequence comprising at least about 2, 3, 4, 5, 6, 7, 8, 9, or 10 amino acids (and up to about 30 amino acids), wherein the amino acid sequence does not comprise a regular secondary structure (e.g., alpha helix, beta strand, 310 helix, beta turn) and/or is a flexible linker. The first linker may comprise a sequence having no or 1 or 2 amino acid substitutions from SEQ ID NO: 10. The first linker may comprise a sequence having no or 1 or 2 amino acid substitutions from SEQ ID NO: 11. The second linker may comprise a sequence having no or 1 or 2 amino acid substitutions from SEQ ID NO: 10. The second linker may comprise a sequence having no or 1 or 2 amino acid substitutions from SEQ ID NO: 11. The first linker may comprise a sequence having no or 1, 2, 3, or 4 amino acid substitutions from SEQ ID NO: 12. The second linker may comprise a sequence having no or 1, 2, 3 or 4 amino acid substitutions from SEQ ID NO: 13. In some embodiments, the GM-CSF molecule comprises a sequence at least about 90%, 91%, 92%, 93%, 94%, 95%, 96%, 97%, 98%, 99%, or 100% identical to SEQ ID NO: 18.

In some embodiments, the GM-CSF molecules comprising a first linker, a GM-CSF peptide, and a second linker (in some cases referred to as a GM-CSF insert) are connected to an antibody variable domain. The GM-CSF insert may be positioned between a first sequence of the antibody variable domain (e.g., a framework 1 of a heavy or light chain) and a second sequence of the antibody variable domain (e.g., a framework 4 of the heavy or light chain, respectively). The first sequence of the antibody variable domain may comprise a sequence having no or 1, 2 or 3 amino acid substitutions from SEQ ID NO: 14. The second sequence of the antibody variable domain may comprise a sequence having no or 1, 2, or 3 amino acid substitutions from SEQ ID NO: 15. The first sequence of the antibody variable domain may comprise a sequence having no or 1, 2 or 3 amino acid substitutions from SEQ ID NO: 32. The second sequence of the antibody variable domain may comprise a sequence having no or 1, 2, or 3 amino acid substitutions from SEQ ID NO: 33.

In some embodiments, the GM-CSF molecules comprise a region at least about 90%, 91%, 92%, 93%, 94%, 95%, 96%, 97%, 98%, 99%, or 100% identical to SEQ ID NO: 42, wherein the region comprises the X5, and the X5 comprises the GM-CSF insert. In some embodiments, the GM-CSF molecules further comprise a region at least about 90%, 91%, 92%, 93%, 94%, 95%, 96%, 97%, 98%, 99%, or 100% identical to SEQ ID NO: 31.

In some embodiments, the GM-CSF molecules comprise a region at least about 90%, 91%, 92%, 93%, 94%, 95%, 96%, 97%, 98%, 99%, or 100% identical to SEQ ID NO: 26, wherein the region comprises the X1, and the X1 comprises the GM-CSF insert. In some embodiments, the GM-CSF molecules further comprise a region at least about 90%, 91%, 92%, 93%, 94%, 95%, 96%, 97%, 98%, 99%, or 100% identical to SEQ ID NO: 2.

In one aspect, provided herein is a GM-CSF molecule comprising a first polypeptide comprising a sequence at least about 90%, 91%, 92%, 93%, 94%, 95%, 96%, 97%, 98%, 99%, or 100% identical to SEQ ID NO: 29, and a second polypeptide comprising a sequence at least about 90%, 91%, 92%, 93%, 94%, 95%, 96%, 97%, 98%, 99%, or 100% identical to SEQ ID NO: 31. In some embodiments, the first polypeptide comprises a sequence at least about 95% identical to SEQ ID NO: 29 and the second polypeptide comprises a sequence at least about 95% identical to SEQ ID NO: 31. In some embodiments, the first polypeptide comprises a sequence at least about 96% identical to SEQ ID NO: 29 and the second polypeptide comprises a sequence at least about 96% identical to SEQ ID NO: 31. In some embodiments, the first polypeptide comprises a sequence at least about 97% identical to SEQ ID NO: 29 and the second polypeptide comprises a sequence at least about 97% identical to SEQ ID NO: 31. In some embodiments, the first polypeptide comprises a sequence at least about 98% identical to SEQ ID NO: 29 and the second polypeptide comprises a sequence at least about 98% identical to SEQ ID NO: 31. In some embodiments, the first polypeptide comprises a sequence at least about 99% identical to SEQ ID NO: 29 and the second polypeptide comprises a sequence at least about 99% identical to SEQ ID NO: 31. In some embodiments, the first polypeptide comprises SEQ ID NO: 29 and a second polypeptide comprises SEQ ID NO: 31.

In one aspect, provided herein is a GM-CSF molecule comprising a first polypeptide comprising a sequence at least about 90%, 91%, 92%, 93%, 94%, 95%, 96%, 97%, 98%, 99%, or 100% identical to SEQ ID NO: 28, and a second polypeptide comprising a sequence at least about 90%, 91%, 92%, 93%, 94%, 95%, 96%, 97%, 98%, 99%, or 100% identical to SEQ ID NO: 30. In some embodiments, the first polypeptide comprises a sequence at least about 95% identical to SEQ ID NO: 28 and the second polypeptide comprises a sequence at least about 95% identical to SEQ ID NO: 30. In some embodiments, the first polypeptide comprises a sequence at least about 96% identical to SEQ ID NO: 28 and the second polypeptide comprises a sequence at least about 96% identical to SEQ ID NO: 30. In some embodiments, the first polypeptide comprises a sequence at least about 97% identical to SEQ ID NO: 28 and the second polypeptide comprises a sequence at least about 97% identical to SEQ ID NO: 30. In some embodiments, the first polypeptide comprises a sequence at least about 98% identical to SEQ ID NO: 28 and the second polypeptide comprises a sequence at least about 98% identical to SEQ ID NO: 30. In some embodiments, the first polypeptide comprises a sequence at least about 99% identical to SEQ ID NO: 28 and the second polypeptide comprises a sequence at least about 99% identical to SEQ ID NO: 30. In some embodiments, the first polypeptide comprises SEQ ID NO: 28 and a second polypeptide comprises SEQ ID NO: 30.

In one aspect, provided herein is a GM-CSF molecule comprising a first polypeptide comprising a sequence at least about 90%, 91%, 92%, 93%, 94%, 95%, 96%, 97%, 98%, 99%, or 100% identical to SEQ ID NO: 2, and a second polypeptide comprising a sequence at least about 90%, 91%, 92%, 93%, 94%, 95%, 96%, 97%, 98%, 99%, or 100% identical to SEQ ID NO: 6. In some embodiments, the first polypeptide comprises a sequence at least about 95% identical to SEQ ID NO: 2 and the second polypeptide comprises a sequence at least about 95% identical to SEQ ID NO: 6. In some embodiments, the first polypeptide comprises a sequence at least about 96% identical to SEQ ID NO: 2 and the second polypeptide comprises a sequence at least about 96% identical to SEQ ID NO: 6. In some embodiments, the first polypeptide comprises a sequence at least about 97% identical to SEQ ID NO: 2 and the second polypeptide comprises a sequence at least about 97% identical to SEQ ID NO: 6. In some embodiments, the first polypeptide comprises a sequence at least about 98% identical to SEQ ID NO: 2 and the second polypeptide comprises a sequence at least about 98% identical to SEQ ID NO: 6. In some embodiments, the first polypeptide comprises a sequence at least about 99% identical to SEQ ID NO: 2 and the second polypeptide comprises a sequence at least about 99% identical to SEQ ID NO: 6. In some embodiments, the first polypeptide comprises SEQ ID NO: 2 and a second polypeptide comprises SEQ ID NO: 6.

In one aspect, provided herein is a GM-CSF molecule comprising a first polypeptide comprising a sequence at least about 90%, 91%, 92%, 93%, 94%, 95%, 96%, 97%, 98%, 99%, or 100% identical to SEQ ID NO: 1, and a second polypeptide comprising a sequence at least about 90%, 91%, 92%, 93%, 94%, 95%, 96%, 97%, 98%, 99%, or 100% identical to SEQ ID NO: 5. In some embodiments, the first polypeptide comprises a sequence at least about 95% identical to SEQ ID NO: 1 and the second polypeptide comprises a sequence at least about 95% identical to SEQ ID NO: 5. In some embodiments, the first polypeptide comprises a sequence at least about 96% identical to SEQ ID NO: 1 and the second polypeptide comprises a sequence at least about 96% identical to SEQ ID NO: 5. In some embodiments, the first polypeptide comprises a sequence at least about 97% identical to SEQ ID NO: 1 and the second polypeptide comprises a sequence at least about 97% identical to SEQ ID NO: 5. In some embodiments, the first polypeptide comprises a sequence at least about 98% identical to SEQ ID NO: 1 and the second polypeptide comprises a sequence at least about 98% identical to SEQ ID NO: 5. In some embodiments, the first polypeptide comprises a sequence at least about 99% identical to SEQ ID NO: 1 and the second polypeptide comprises a sequence at least about 99% identical to SEQ ID NO: 5. In some embodiments, the first polypeptide comprises SEQ ID NO: 1 and a second polypeptide comprises SEQ ID NO: 5.

In one aspect, provided herein is a GM-CSF molecule comprising a first polypeptide comprising a sequence at least about 90%, 91%, 92%, 93%, 94%, 95%, 96%, 97%, 98%, 99%, or 100% identical to SEQ ID NO: 45, and a second polypeptide comprising a sequence at least about 90%, 91%, 92%, 93%, 94%, 95%, 96%, 97%, 98%, 99%, or 100% identical to SEQ ID NO: 46. In one aspect, provided herein is a GM-CSF molecule comprising a first polypeptide comprising a sequence at least about 90%, 91%, 92%, 93%, 94%, 95%, 96%, 97%, 98%, 99%, or 100% identical to SEQ ID NO: 47, and a second polypeptide comprising a sequence at least about 90%, 91%, 92%, 93%, 94%, 95%, 96%, 97%, 98%, 99%, or 100% identical to SEQ ID NO: 48. In one aspect, provided herein is a GM-CSF molecule comprising a first polypeptide comprising a sequence at least about 90%, 91%, 92%, 93%, 94%, 95%, 96%, 97%, 98%, 99%, or 100% identical to SEQ ID NO: 49, and a second polypeptide comprising a sequence at least about 90%, 91%, 92%, 93%, 94%, 95%, 96%, 97%, 98%, 99%, or 100% identical to SEQ ID NO: 50. In one aspect, provided herein is a GM-CSF molecule comprising a first polypeptide comprising a sequence at least about 90%, 91%, 92%, 93%, 94%, 95%, 96%, 97%, 98%, 99%, or 100% identical to SEQ ID NO: 51, and a second polypeptide comprising a sequence at least about 90%, 91%, 92%, 93%, 94%, 95%, 96%, 97%, 98%, 99%, or 100% identical to SEQ ID NO: 52. In one aspect, provided herein is a GM-CSF molecule comprising a first polypeptide comprising a sequence at least about 90%, 91%, 92%, 93%, 94%, 95%, 96%, 97%, 98%, 99%, or 100% identical to SEQ ID NO: 53, and a second polypeptide comprising a sequence at least about 90%, 91%, 92%, 93%, 94%, 95%, 96%, 97%, 98%, 99%, or 100% identical to SEQ ID NO: 54. In one aspect, provided herein is a GM-CSF molecule comprising a first polypeptide comprising a sequence at least about 90%, 91%, 92%, 93%, 94%, 95%, 96%, 97%, 98%, 99%, or 100% identical to SEQ ID NO: 55, and a second polypeptide comprising a sequence at least about 90%, 91%, 92%, 93%, 94%, 95%, 96%, 97%, 98%, 99%, or 100% identical to SEQ ID NO: 56. In one aspect, provided herein is a GM-CSF molecule comprising a first polypeptide comprising a sequence at least about 90%, 91%, 92%, 93%, 94%, 95%, 96%, 97%, 98%, 99%, or 100% identical to SEQ ID NO: 57, and a second polypeptide comprising a sequence at least about 90%, 91%, 92%, 93%, 94%, 95%, 96%, 97%, 98%, 99%, or 100% identical to SEQ ID NO: 58. In one aspect, provided herein is a GM-CSF molecule comprising a first polypeptide comprising a sequence at least about 90%, 91%, 92%, 93%, 94%, 95%, 96%, 97%, 98%, 99%, or 100% identical to SEQ ID NO: 59, and a second polypeptide comprising a sequence at least about 90%, 91%, 92%, 93%, 94%, 95%, 96%, 97%, 98%, 99%, or 100% identical to SEQ ID NO: 60. In one aspect, provided herein is a GM-CSF molecule comprising a first polypeptide comprising a sequence at least about 90%, 91%, 92%, 93%, 94%, 95%, 96%, 97%, 98%, 99%, or 100% identical to SEQ ID NO: 61, and a second polypeptide comprising a sequence at least about 90%, 91%, 92%, 93%, 94%, 95%, 96%, 97%, 98%, 99%, or 100% identical to SEQ ID NO: 62. In one aspect, provided herein is a GM-CSF molecule comprising a first polypeptide comprising a sequence at least about 90%, 91%, 92%, 93%, 94%, 95%, 96%, 97%, 98%, 99%, or 100% identical to SEQ ID NO: 63, and a second polypeptide comprising a sequence at least about 90%, 91%, 92%, 93%, 94%, 95%, 96%, 97%, 98%, 99%, or 100% identical to SEQ ID NO: 64.

In one aspect, provided herein is a GM-CSF molecule comprising a sequence at least about 90%, 91%, 92%, 93%, 94%, 95%, 96%, 97%, 98%, 99%, or 100% identical to SEQ ID NO: 67. In one aspect, provided herein is a GM-CSF molecule comprising a sequence at least about 90%, 91%, 92%, 93%, 94%, 95%, 96%, 97%, 98%, 99%, or 100% identical to SEQ ID NO: 68.

In one aspect, provided herein is a GM-CSF molecule encoded by a first nucleic acid comprising a sequence at least about 90%, 91%, 92%, 93%, 94%, 95%, 96%, 97%, 98%, 99%, or 100% identical to SEQ ID NO: 69, and a second nucleic acid comprising a sequence at least about 90%, 91%, 92%, 93%, 94%, 95%, 96%, 97%, 98%, 99%, or 100% identical to SEQ ID NO: 70. In one aspect, provided herein is a GM-CSF molecule encoded by a first nucleic acid comprising a sequence at least about 90%, 91%, 92%, 93%, 94%, 95%, 96%, 97%, 98%, 99%, or 100% identical to SEQ ID NO: 71, and a second nucleic acid comprising a sequence at least about 90%, 91%, 92%, 93%, 94%, 95%, 96%, 97%, 98%, 99%, or 100% identical to SEQ ID NO: 72. In one aspect, provided herein is a GM-CSF molecule encoded by a first nucleic acid comprising a sequence at least about 90%, 91%, 92%, 93%, 94%, 95%, 96%, 97%, 98%, 99%, or 100% identical to SEQ ID NO: 73, and a second nucleic acid comprising a sequence at least about 90%, 91%, 92%, 93%, 94%, 95%, 96%, 97%, 98%, 99%, or 100% identical to SEQ ID NO: 74. In one aspect, provided herein is a GM-CSF molecule encoded by a first nucleic acid comprising a sequence at least about 90%, 91%, 92%, 93%, 94%, 95%, 96%, 97%, 98%, 99%, or 100% identical to SEQ ID NO: 75, and a second nucleic acid comprising a sequence at least about 90%, 91%, 92%, 93%, 94%, 95%, 96%, 97%, 98%, 99%, or 100% identical to SEQ ID NO: 76.

In some embodiments, a GM-CSF molecule comprises one or more sequences from SEQ ID NOS: 1-76. In some embodiments, a GM-CSF molecule comprises a sequence from Table 4. Antibody abbreviations in Table 4 include: “HC” for heavy chain, “VH” for variable region of the heavy chain, “Fc” for fragment crystallizable region, “LC” for light chain, “VL” for variable region of the light chain, “CDR3L” for complementarity determining region 3 of the light chain, “CDR3H” for complementarity determining region 3 of the heavy chain, and “FR” for framework.

### Scaffolds

In one aspect, GM-CSF molecules are provided herein comprising a GM-CSF peptide connected to a scaffold. The scaffold may increase the half-life of the GM-CSF peptide. Measurement of half-life may be measured using the experiments detailed in the examples provided herein, or methods readily available in the art. The half-life may be increased by at least about 10%, 20%, 30%, 40%, 50%, 100%, 150%, 200%, 250%, 300%, 350%, 400%, 450%, 500%, 600%, 700%, 800%, 900%, or 100% as compared to GM-CSF without the fusion, e.g., recombinant GM-CSF such as sargramostim. Accordingly, provided herein are long-acting GM-CSF molecules comprising increased half-life as compared to GM-CSF peptide alone.

In some embodiments, the scaffold comprises one or more antibody portions. The antibody portion may comprise an entire antibody molecule or any polypeptide comprising fragment of an antibody including, but not limited to, heavy chain, light chain, variable domain, variable light chain region (VL), variable heavy chain region (VH), constant domain (e.g., CH1, CH2, CH3, and/or CL), complementarity determining region (CDR, e.g., CDRH1, CDRH2, CDRH3, CDRL1, CDRL2, and/or CDRL3), framework region (e.g., FRH1, FRH2, FRH3, FRH4, FRL1, FRL2, FRL3, and/or FRL4), antigen binding fragment, single domain antibody, fragment antigen binding (Fab) region, Fab′, F(ab′)2, F(ab′)3, Fab′, fragment crystallizable (Fc) region, single chain variable fragment (scFv), di-scFv, single domain antibody, trifunctional antibody, chemically linked F(ab′)2, and any combination thereof The antibody portion may comprise a heavy chain and a light chain connected by a linker or by a disulfide bond. In some cases, the antibody portion comprises a variable domain that has been modified or otherwise engineered to reduce or eliminate antigen binding The antigen binding may be reduced or eliminated by mutation, e.g., mutating the CDR3 of the light and/or heavy chain. The antibody may be derived from any type known to one of skill in the art including, but not limited to, IgA, IgD, IgE, IgG, IgM, IgY, and IgW.

As used herein, an antibody variable domain is not limited to an antibody fragment capable of binding an antigen, but also includes antibody fragments derived from an antibody fragment capable of binding an antigen, wherein the derivatization comprises reducing or eliminating antigen binding. In some such cases, the amino acid length of the antibody binding fragment that has been derivatized to reduce or eliminate antigen binding may be the same, or within about 10%-120% of, the amino acid length of the antigen binding fragment from which it was derived (i.e. the antigen binding fragment that binds to an antigen). In some cases, an antibody variable domain is an antibody region comprising the CDR1, CDR2, and CDR3 of an antibody heavy chain, and the CDR1, CDR2, and CDR3 of an antibody light chain; wherein one or more of the CDRs has been mutated or otherwise altered in amino acid sequence identity to reduce or eliminate antigen binding as compared to the non-mutated or altered antibody. For GM-CSF molecules where a GM- CSF peptide is positioned within a CDR, an antibody variable domain may comprise an antibody region comprising the CDR1, CDR2, and CDR3 of an antibody heavy chain, and the CDR1, CDR2, and CDR3 of an antibody light chain; wherein one or more of the CDRs has been mutated or otherwise altered in amino acid sequence identity to reduce or eliminate antigen binding. In some cases, the modification is positioned of the GM-CSF peptide within a CDR region, e.g., by replacing 1, 2, 3, 4, 5, or all CDR amino acids with the GM-CSF peptide.

The antibody portion may be modified from a trastuzumab antibody comprising a heavy chain variable region comprising SEQ ID NO: 44 and a light chain variable region comprising SEQ ID NO: 31. The antibody portion may be modified from a palivizumab antibody comprising a heavy chain variable region comprising SEQ ID NO: 65 and a light chain variable region comprising SEQ ID NO: 17. The antibody portion may be modified by insertion of a GM-CSF peptide into the heavy or light chain sequence. The GM-CSF peptide may replace one or more amino acids of the heavy or light chain sequence. The GM-CSF may be positioned within a CDR of the heavy or light chain sequence. The CDR may be a CDR3. The antibody portion may be connected to a GM-CSF peptide at the amino or carboxy terminus of the heavy or light chain sequence. In some embodiments, the antibody portion is modified to reduce antigen binding. For instance, the palivizumab heavy chain CDR3 may be modified to replace NWY with FGG.

In some embodiments, the antibody portion comprises SEQ ID NO: 14. In some embodiments, the antibody portion comprises SEQ ID NO: 15. The antibody portion may comprise SEQ ID NOS: 14 and 15. The GM-CSF peptide may be positioned between SEQ ID NOS: 14 and 15. In some embodiments, the antibody portion comprises SEQ ID NO: 32. In some embodiments, the antibody portion comprises SEQ ID NO: 33. The antibody portion may comprise SEQ ID NOS: 32 and 33. The GM-CSF peptide may be positioned between SEQ ID NOS: 32 and 33.

In some cases, a scaffold comprises “at least a portion” of an antibody or antibody fragment. In certain embodiments, “at least a portion” indicates that at least about 80%, 85%, 90%, 91%, 92%, 93%, 94%, 95%, 96%, 97%, 98%, or 99% of the length of the antibody or antibody fragment is present in the composition at a sequence identity of at least about 90%, 95%, 96%, 97%, 98%, 99%, or 100%. In some embodiments, a scaffold comprising at least a portion of heavy chain having SEQ ID NO: 44 (120 amino acids), comprises at least about 96 amino acids (80%), 102 amino acids (85%), 108 amino acids (90%) or 114 amino acids (95%) of SEQ ID NO: 44 with at least about 90%, 95%, 96%, 97%, 98%, 99%, or 100% sequence identity. The “at least a portion” of may be a continuous amino acid sequence, or the sum of two continuous amino acid sequences that are separated by a GM-CSF peptide. For example, GM-CSF molecule comprising at least a portion of an antibody variable domain may comprise a first continuous amino acid sequence of the antibody variable domain, a GM-CSF peptide, and a second continuous amino acid sequence of the antibody variable domain, where the first and second continuous amino acid sequences of the antibody variable domain add up to at least about 80%, 85%, 90%, 91%, 92%, 93%, 94%, 95%, 96%, 97%, 98%, or 99% of the sequence length of an antibody variable domain. In some cases, a GM-CSF molecule comprises at least a portion of an antibody or an antibody fragment selected from: an antibody variable domain and/or an antigen binding fragment (e.g., a fragment comprising a CDR1, CDR2, and CDR3 of an antibody heavy chain and/or antibody light chain); wherein the molecule comprises a first antibody or antibody fragment region, a GM-CSF peptide, and a second antibody or antibody fragment region; and wherein the “at least a portion” indicates that the sum of the length of the first antibody or antibody fragment region and the length of the second antibody or antibody fragment region is at least about 80%, 85%, 90%, 91%, 92%, 93%, 94%, 95%, 96%, 97%, 98%, or 99% of the length of the antibody variable domain and/or antigen binding fragment.

The antibody portion may comprise an antibody sequence from a trastuzumab antibody. The antibody portion may comprise an amino acid sequence that is identical to or at least about 50%, 60%, 65%, 70%, 75%, 80%, 85%, 90%, 91%, 92%, 93%, 94%, 95%, 96%, 97%, 98%, 99%, or 100% identical to at least a portion of a trastuzumab antibody. For example, the antibody portion comprises an amino acid sequence at least 80%, 85%, 90%, 91%, 92%, 93%, 94%, 95%, 96%, 97%, 98%, 99%, or 100% identical to a sequence selected from one or more of: SEQ ID NOS: 30-35, 37-39, and 44. As another example, the antibody portion comprises at least about 10 contiguous amino acids of a sequence selected from one or more of: 30-35, 37-39, and 44.

The antibody portion may comprise an antibody sequence from an anti-Her2 antibody. The antibody portion may comprise at least a portion of an anti-Her2 antibody. The antibody portion may comprise an amino acid sequence that is identical to or at least about 50%, 60%, 65%, 70%, 75%, 80%, 85%, 90%, 92%, 95%, or 97% or more identical to at least a portion of an anti-Her2 antibody.

In some cases, the antibody portion comprises a palivizumab antibody sequence. The antibody portion may comprise an amino acid sequence that is identical to or at least about 50%, 60%, 65%, 70%, 75%, 80%, 85%, 90%, 91%, 92%, 93%, 94%, 95%, 96%, 97%, 98%, 99%, or 100% identical to at least a portion of a palivizumab antibody. For example, the antibody portion comprises an amino acid sequence at least 80%, 85%, 90%, 91%, 92%, 93%, 94%, 95%, 96%, 97%, 98%, 99%, or 100% identical to a sequence selected from one or more of: SEQ ID NOS: 14, 15, 17, 65, and 19-23. As another example, the antibody portion comprises at least about 10 contiguous amino acids of a sequence selected from one or more of: 14, 15, 17, 65, and 19-23.

In some embodiments, an antibody scaffold provided herein is not specific for or has reduced binding to an antigen. For example, an antibody scaffold provided herein may not bind to an antigen or may bind to an antigen with an affinity weaker than about 10 M⁻², 10 M⁻³, or 10 M⁻⁵, e.g., as determined by an assay described in an example herein. An exemplary antibody comprises six CDRs, wherein one or more of the CDRs is a modified CDR comprising one or more amino acid additions, substitutions, or deletions that reduce or eliminate antigen binding. In some cases, a CDR is modified by insertion of a therapeutic peptide such as GM-CSF. In exemplary embodiments, the GM-CSF is inserted in a CDR3 of a heavy or light chain. Exemplary GM-CSF molecules provided herein comprise a GM-CSF peptide positioned within a heavy chain CDR3 of an antibody variable domain, wherein the antibody has no or reduced antigen binding as compared to the antibody without the GM-CSF fusion. The GM-CSF may replace one, two, three, four, five, or all amino acids of a CDR. The antibody may comprise a trastuzumab antibody variable domain scaffold modified by insertion of GM-CSF within a CDR. In some cases, insertion of GM-CSF within the CDR3 of trastuzumab reduces antigen binding. An antibody comprising an insertion may further comprise one or more amino acid deletions, for instance, if the insertion replaces one or more amino acids of the CDR. In some embodiments, a CDR sequence is mutated to reduced antigen binding. In some embodiments, a heavy chain CDR3 of palivizumab comprises SMITX(i)X(ii)X(iii)FDV (SEQ ID NO: 66), wherein X(i) is selected from F, A, G, and P; X(ii) is selected from G, A, S, T, and P; and X(iii) is selected from G, A, V, L, and P. In some embodiments, X(i) is F. In some embodiments, X(ii) is G. In some embodiments, X(ii) is A. In some embodiments, X(iii) is G. As an example, the heavy chain CDR3 of the antibody palivizumab is mutation to replace NWY with FGG, which reduces binding to RSV-F as compared to non-mutated palivizumab. As another example, the heavy chain CDR3 of the antibody trastuzumab is mutated to remove about 1, 2, 3, 4, 5, or all CDR3 amino acids, which reduces binding to Her2 as compared to non-mutated trastuzumab. In some cases one or more amino acids of the CDR3 of trastuzumab is replaced with a GM-CSF peptide or GM-CSF insert.

In some embodiments, the antibody portion is not specific for a mammalian target. In some embodiments, the antibody is an anti-viral antibody. In some embodiments, the antibody is an anti-bacterial antibody. In some embodiments, the antibody is an anti-parasitic antibody. In some embodiments, the antibody is an anti-fungal antibody. In some embodiments, the antibody portion is derived from an antibody vaccine.

In some embodiments, the antibody portion comprises an antibody sequence from, but not limited to, actoxumab, bezlotoxumab, CR6261, edobacomab, efungumab, exbivirumab, felvizumab, foravirumab, ibalizumab (TMB-355, TNX-355), libivirumab, motavizumab, nebacumab, pagibaximab, palivizumab, panobacumab, rafivirumab, raxibacumab, regavirumab, sevirumab (MSL-109), suvizumab, tefibazumab, tuvirumab, and urtoxazumab.

In some embodiments, the antibody portion comprises an antibody sequence from antibodies targeting Clostridium difficile, Orthomyxoviruses (Influenzavirus A, Influenzavirus B, Influenzavirus C, Isavirus, Thogotovirus), Escherichia coli, Candida, Rabies, Human Immunodeficiency Virus, Hepatitis, Staphylococcus, Respiratory Syncytial Virus, Pseudomonas aeruginosa, Bacillus anthracis, Cytomegalovirus, or Staphylococcus aureus.

The antibody portion may comprise an antibody sequence from an anti-viral antibody. The anti-viral antibody may be directed against an epitope of a viral protein. The anti-viral antibody may target one or more viruses including, but not limited to, Adenoviruses, Herpesviruses, Poxviruses, Parvoviruses, Reoviruses, Picornaviruses, Togaviruses, Orthomyxoviruses, Rhabdoviruses, Retroviruses and Hepadnaviruses. The viral protein may be from a respiratory syncytial virus. The viral protein may be an F protein of the respiratory syncytiral virus. The epitope may be in the A antigenic site of the F protein. The anti-viral antibody may be based on or derived from palivizumab. The antibody may be based on or derived from an anti-viral vaccine. The anti-viral antibody may be based on or derived from exbivirumab, foravirumab, libivirumab, rafivirumab, regavirumab, sevirumab, tuvirumab, felvizumab, motavizumab, palivizumab, and/or suvizumab.

The antibody portion may comprise an antibody sequence from an anti-viral antibody G. The antibody portion may comprise at least a portion of an anti-viral antibody G. The antibody portion may comprise an amino acid sequence that is at least about 50%, 60%, 65%, 70%, 75%, 80%, 85%, 90%, 92%, 95%, or 97% or more identical to at least a portion of an anti-viral antibody G. In some embodiments the antibody portion comprises an amino acid sequence of an anti-viral antibody M.

The antibody portion may comprise an antibody sequence from an exbivirumab, foravirumab, libivirumab, rafivirumab, regavirumab, sevirumab, tuvirumab, felvizumab, motavizumab, palivizumab, and/or suvizumab antibody. The antibody portion may comprise at least a portion of an exbivirumab, foravirumab, libivirumab, rafivirumab, regavirumab, sevirumab, tuvirumab, felvizumab, motavizumab, palivizumab, and/or suvizumab antibody. The antibody portion may comprise an antibody sequence that is identical to or at least about 50%, 60%, 65%, 70%, 75%, 80%, 85%, 90%, 92%, 95%, or 97% or more identical to at least a portion of an exbivirumab, foravirumab, libivirumab, rafivirumab, regavirumab, sevirumab, tuvirumab, felvizumab, motavizumab, palivizumab, and/or suvizumab antibody.

The antibody portion may comprise an antibody sequence from an anti-bacterial antibody. The anti-bacterial antibody may be directed against an epitope of a bacterial protein. The anti-bacterial antibody may target bacteria including, but not limited to, Acetobacter aurantius, Agrobacterium radiobacter, Anaplasma phagocytophilum, Azorhizobium caulinodans, Bacillus anthracis, Bacillus brevis, Bacillus cereus, Bacillus subtilis, Bacteroides fragilis, Bacteroides gingivalis, Bacteroides melaninogenicus, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia, Calymmatobacterium granulomatis, Campylobacter coli, Campylobacter fetus, Campylobacter jejuni, Campylobacter pylori, Chlamydia trachomatis, Chlamydophila pneumoniae, Chlamydophila psittaci, Clostridium botulinum, Clostridium difficile, Corynebacterium diphtheriae, Corynebacterium fusiforme, Coxiella burnetii, Enterobacter cloacae, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus influenzae, Haemophilus parainfluenzae, Haemophilus pertussis, Haemophilus vaginalis, Helicobacter pylori, Klebsiella pneumoniae, Lactobacillus acidophilus, Lactococcus lactis, Legionella pneumophila, Listeria monocytogenes, Methanobacterium extroquens, Microbacterium multiforme, Micrococcus luteus, Moraxella catarrhalis, Mycobacterium phlei, Mycobacterium smegmatis, Mycobacterium tuberculosis, Mycoplasma genitalium, Mycoplasma hominis, Mycoplasma pneumonie, Neisseria gonorrhoeae, Neisseria meningitidis, Pasteurella multocida, Pasteurella tularensis, Peptostreptococcus, Porphyromonas gingivalis, Prevotella melaninogenica, Pseudomonas aeruginosa, Rhizobium radiobacter, Rickettsia rickettsii, Rothia dentocariosa, Salmonella enteritidis, Salmonella typhi, Salmonella typhimurium, Shigella dysenteriae, Staphylococcus aureus, Staphylococcus epidermidis, Stenotrophomonas maltophilia, Streptococcus pneumoniae, Streptococcus pyogenes, Treponema pallidum, Treponema denticola, Vibrio cholerae, Vibrio comma, Vibrio parahaemolyticus, Vibrio vulnificus, Yersinia enterocolitica and Yersinia pseudotuberculosis. The antibody may be based on or derived from a bacterial vaccine. The anti-viral antibody may be based on or derived from nebacumab, panobacumab, raxibacumab, edobacomab, pagibaximab, and/or tefibazumab.

The antibody portion may comprise an antibody sequence from an anti-bacterial antibody G. The antibody portion may comprise at least a portion of an anti-bacterial antibody G. The antibody portion may comprise an amino acid sequence that is identical to or at least about 50%, 60%, 65%, 70%, 75%, 80%, 85%, 90%, 92%, 95%, or 97% or more identical to at least a portion of an anti-bacterial antibody G. In some embodiments the antibody portion comprises an amino acid sequence based on or derived from an anti-bacterial antibody M.

The antibody portion may comprise an antibody sequence from a nebacumab, panobacumab, raxibacumab, edobacomab, pagibaximab, and/or tefibazumab antibody. The antibody portion may comprise an amino acid sequence that is identical to or at least about 50%, 60%, 65%, 70%, 75%, 80%, 85%, 90%, 92%, 95%, or 97% or more identical to at least a portion of a nebacumab, panobacumab, raxibacumab, edobacomab, pagibaximab, and/or tefibazumab antibody.

The antibody portion may comprise an antibody sequence from an anti-parasitic antibody. The anti-parasitic antibody may be directed against an epitope of a parasite protein. The anti-parasitic antibody may target parasites or parasite proteins including, but not limited to parasites Acanthamoeba, Balamuthia mandrillaris, Babesia (B. divergens, B. bigemina, B. equi, B. microfti, B. duncani), Balantidium coli, Blastocystis, Cryptosporidium, Dientamoeba fragilis, Entamoeba histolytica, Giardia lamblia, Isospora belli, Leishmania, Naegleria fowleri, Plasmodium falciparum, Plasmodium vivax, Plasmodium ovale curtisi, Plasmodium ovale wallikeri, Plasmodium malariae, Plasmodium knowlesi, Rhinosporidium seeberi, Sarcocystis bovihominis, Sarcocystis suihominis, Toxoplasma gondii, Trichomonas vaginalis, Trypanosoma brucei, Trypanosoma cruzi, Cestoda, Taenia multiceps, Diphyllobothrium latum, Echinococcus granulosus, Echinococcus multilocularis, Echinococcus vogeli, Echinococcus oligarthrus, Hymenolepis nana, Hymenolepis diminuta, Taenia saginata, Taenia solium, Bertiella mucronata, Bertiella studeri, Spirometra erinaceieuropaei, Clonorchis sinensis; Clonorchis viverrini, Dicrocoelium dendriticum, Fasciola hepatica, Fasciola gigantica, Fasciolopsis buski, Gnathostoma spinigerum, Gnathostoma hispidum, Metagonimus yokogawai, Opisthorchis viverrini, Opisthorchis felineus, Clonorchis sinensis, Paragonimus westermani; Paragonimus africanus; Paragonimus caliensis; Paragonimus kellicotti; Paragonimus skrjabini; Paragonimus uterobilateralis, Schistosoma sp., Schistosoma mansoni, Schistosoma haematobium, Schistosoma japonicum, Schistosoma mekongi, Echinostoma echinatum, Trichobilharzia regenti, Schistosomatidae, Ancylostoma duodenale, Necator americanus, Angiostrongylus costaricensis, Anisakis, Ascaris sp. Ascaris lumbricoides, Baylisascaris procyonis, Brugia malayi, Brugia timori, Dioctophyme renale, Dracunculus medinensis, Enterobius vermicularis, Enterobius gregorii, Halicephalobus gingivalis, Loa filaria, Mansonella streptocerca, Onchocerca volvulus, Strongyloides stercoralis, Thelazia californiensis, Thelazia callipaeda, Toxocara canis, Toxocara cati, Trichinella spiralis, Trichinella britovi, Trichinella nelsoni, Trichinella nativa, Trichuris trichiura, Trichuris vulpis, Wuchereria bancrofti, Archiacanthocephala, Moniliformis, Linguatula serrata, Oestroidea, Calliphoridae, Sarcophagidae, Tunga penetrans, Dermatobia hominis, Ixodidae, Argasidae, Cimex lectularius, Pediculus humanus, Pediculus humanus corporis, Pthirus pubis, Demodex folliculorum/brevis/canis, Sarcoptes scabiei, Cochliomyia hominivorax, and Pulex irritans.

The antibody portion may comprise an antibody sequence from an anti-parasitic antibody G. The antibody portion may comprise at least a portion of an anti-parasitic antibody G. The antibody portion may comprise an amino acid sequence that is identical to or at least about 50%, 60%, 65%, 70%, 75%, 80%, 85%, 90%, 92%, 95%, or 97% or more identical to at least a portion of an anti-parasitic antibody G.

The antibody portion may comprise an antibody sequence from an anti-fungal antibody. The anti-bacterial antibody may be directed against an epitope of a fungal protein. The anti-fungal antibody may target fungi or fungal proteins including, but not limited to Cryptococcus neoformans, Cryptococcus gattii, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Schizosaccharomyces pombe, Saccharomyces cerevisiae, Brettanomyces bruxellensis, Candida stellata, Schizosaccharomyces pombe, Torulaspora delbrueckii, Zygosaccharomyces bailii, Yarrowia hpolytica, Saccharomyces exiguus and Pichia pastoris. The anti-fungal antibody may be based on or derived from efungumab.

The antibody portion may comprise an antibody sequence from an anti-fungal antibody G. The antibody portion may comprise at least a portion of an anti-fungal antibody G. The antibody portion may comprise an amino acid sequence that is identical to or at least about 50%, 60%, 65%, 70%, 75%, 80%, 85%, 90%, 92%, 95%, or 97% or more identical to at least a portion of an anti-fungal antibody G.

The antibody portion may comprise an antibody sequence from an anti-cancer antibody. Examples of anti-cancer antibody include, but are not limited to, abciximab, adalimumab, alemtuzumab, basiliximab, belimumab, bevacizumab, brentuximab, canakinumab, certolizumab, cetuximab, daclizumab, denosumab, eculizumab, efalizumab, gemtuzumab, golimumab, ibritumomab, infliximab, ipilimumab, muromonab-cd3, natalizumab, ofatumumab, omalizumab, palivizumab, panitumumab, ranibizumab, rituximab, tocilizumab, tositumomab, trastuzumab.

The antibody portion may comprise at least a portion of a human antibody. The antibody portion may comprise at least a portion of a humanized antibody. The antibody portion may comprise at least a portion of a chimeric antibody. The antibody portion may be based on or derived from a human antibody. The antibody portion may be based on or derived from a humanized antibody. The antibody portion may be based on or derived from a chimeric antibody. The antibody portion may be based on or derived from a monoclonal antibody. The antibody portion may be based on or derived from a polyclonal antibody. The antibody portion may comprise at least a portion of an antibody from a mammal, avian, reptile, amphibian, or a combination thereof The mammal may be a human. The mammal may be a non-human primate. The mammal may be a dog, cat, sheep, goat, cow, rabbit, rat or mouse.

In one aspect, provided herein are antibody variable domains comprising: (a) a light chain comprising (i) a CDRL1 comprising a sequence having no or about 1, 2, or 3 amino acid substitutions or deletions from SEQ ID NO: 22, (ii) a CDRL2 comprising a sequence having no or about 1, 2, or 3 amino acid substitutions or deletions from SEQ ID NO: 23, and (iii) a CDRL3 comprising a sequence having no or about 1, 2, or 3 amino acid substitutions or deletions from SEQ ID NO: 16 or 77; and a (b) a heavy chain comprising (i) a CDRH1 comprising a sequence having no or about 1, 2, or 3 amino acid substitutions or deletions from SEQ ID NO: 19, (ii) a CDRH2 comprising a sequence having no or about 1, 2, or 3 amino acid substitutions or deletions from SEQ ID NO: 20, and (iii) a CDRH3 comprising a sequence having no or about 1, 2, or 3 amino acid substitutions or deletions from SEQ ID NO: 21. In some embodiments, the CDRL3 comprises a sequence having no or about 1, 2, 3, 4, or 5 amino acid substitutions or deletions from SEQ ID NO: 24. In some embodiments, the antibody variable domains comprise a light chain comprising SEQ ID NOS: 22, 23, and 16; and a heavy chain comprising SEQ ID NOS: 19-21. In some embodiments, the antibody variable domains comprise a light chain comprising SEQ ID NOS: 22, 23, and 77; and a heavy chain comprising SEQ ID NOS: 19-21. In some embodiments, the antibody variable domains comprise a light chain comprising SEQ ID NOS: 22, 23, and 24; and a heavy chain comprising SEQ ID NOS: 19-21. Further provided are antibodies comprising the antibody variable domain and further comprising a Fc region comprising reduced effector function as compared to human IgG (SEQ ID NO: 25). The reduced effector function may be reduced ADCC and/or reduced CDC. The Fc region may comprise a sequence at least about 90%, 91%, 92%, 93%, 94%, 95%, 96%, 97%, 98%, 99%, or 100% identical to SEQ ID NO: 3. In some embodiments, the Fc region comprises a modified human IgG1 comprising E233P, L234V, L235A, ΔG236, A327G, A330S, P331S, per Kabat numbering.

In one aspect, provided herein are antibody variable domains comprising: (a) a light chain comprising (i) a CDRL1 comprising a sequence having no or about 1, 2, or 3 amino acid substitutions or deletions from SEQ ID NO: 37, (ii) a CDRL2 comprising a sequence having no or about 1, 2, or 3 amino acid substitutions or deletions from SEQ ID NO: 38, and (iii) a CDRL3 comprising a sequence having no or about 1, 2, or 3 amino acid substitutions or deletions from SEQ ID NO: 39; and a (b) a heavy chain comprising (i) a CDRH1 comprising a sequence having no or about 1, 2, or 3 amino acid substitutions or deletions from SEQ ID NO: 34, (ii) a CDRH2 comprising a sequence having no or about 1, 2, or 3 amino acid substitutions or deletions from SEQ ID NO: 35, and (iii) a CDRH3 comprising a sequence having no or about 1, 2, or 3 amino acid substitutions or deletions from SEQ ID NO: 16. In some embodiments, the CDRH3 comprises a sequence having no or about 1, 2, 3, 4, or 5 amino acid substitutions or deletions from SEQ ID NO: 36. In some embodiments, the antibody variable domains comprise a light chain comprising SEQ ID NOS: 37-39; and a heavy chain comprising SEQ ID NOS: 34, 35, and 16. In some embodiments, the antibody variable domains comprise a light chain comprising SEQ ID NOS: 37-39; and a heavy chain comprising SEQ ID NOS: 34-36. Further provided are antibodies comprising the antibody variable domain and further comprising a Fc region comprising reduced effector function as compared to human IgG (SEQ ID NO: 25). The reduced effector function may be reduced ADCC and/or reduced CDC. The Fc region may comprise a sequence at least about 90%, 91%, 92%, 93%, 94%, 95%, 96%, 97%, 98%, 99%, or 100% identical to SEQ ID NO: 3. In some embodiments, the Fc region comprises a modified human IgG1 comprising E233P, L234V, L235A, ΔG236, A327G, A330S, P331S, per Kabat numbering.

In one aspect, provided herein are antibody variable domains comprising: (a) a light chain comprising (i) a CDRL1 comprising a sequence having no or about 1, 2, or 3 amino acid substitutions or deletions from SEQ ID NO: 37, (ii) a CDRL2 comprising a sequence having no or about 1, 2, or 3 amino acid substitutions or deletions from SEQ ID NO: 38, and (iii) a CDRL3 comprising a sequence having no or about 1, 2, or 3 amino acid substitutions or deletions from SEQ ID NO: 39; and a (b) a heavy chain comprising (i) a CDRH1 comprising a sequence having no or about 1, 2, or 3 amino acid substitutions or deletions from SEQ ID NO: 34, (ii) a CDRH2 comprising a sequence having no or about 1, 2, or 3 amino acid substitutions or deletions from SEQ ID NO: 35, and (iii) a CDRH3 comprising a sequence having no or about 1, 2, or 3 amino acid substitutions or deletions from SEQ ID NO: 77. In some embodiments, the CDRH3 comprises a sequence having no or about 1, 2, 3, 4, or 5 amino acid substitutions or deletions from SEQ ID NO: 36. In some embodiments, the antibody variable domains comprise a light chain comprising SEQ ID NOS: 37-39; and a heavy chain comprising SEQ ID NOS: 34, 35, and 77. Further provided are antibodies comprising the antibody variable domain and further comprising a Fc region comprising reduced effector function as compared to human IgG (SEQ ID NO: 25). The reduced effector function may be reduced ADCC and/or reduced CDC. The Fc region may comprise a sequence at least about 90%, 91%, 92%, 93%, 94%, 95%, 96%, 97%, 98%, 99%, or 100% identical to SEQ ID NO: 3. In some embodiments, the Fc region comprises a modified human IgG1 comprising E233P, L234V, L235A, ΔG236, A327G, A330S, P331S, per Kabat numbering.

Reduced Effector Function

Antibody scaffolds provided herein may comprise one or more amino acid additions, deletions, and/or substitutions to wild-type IgG Fc region to reduce binding to an effector molecule as compared to wild-type IgG. The antibody may have reduced antibody-dependent cellular cytotoxicity (ADCC) and/or complement-dependent cytotoxicity. As an example, the scaffold comprises a IgG1 Fc region comprising one or more of the following mutations: E233P, L234V, L235A, ΔG236, A327G, A330S, and P331S. In some embodiments, an antibody comprises a constant region comprising an amino acid sequence at least about 90%, 91%, 92%, 93%, 94%, 95%, 96%, 97%, 98%, 99% or 100% identical to 25. In some embodiments, an antibody comprises a CH1 domain comprising an amino acid sequence at least about 90%, 91%, 92%, 93%, 94%, 95%, 96%, 97%, 98%, 99% or 100% identical to 3. In some embodiments, an antibody comprises a Fc region comprising an amino acid sequence at least about 90%, 91%, 92%, 93%, 94%, 95%, 96%, 97%, 98%, 99% or 100% identical to 4.

Linkers

GM-CSF molecules may comprise a GM-CSF peptide connected to a scaffold via one or more linkers. In some cases a linker molecule comprises a linker peptide comprising a secondary structure. The secondary structure may be an alpha-helix. Exemplary alpha helical peptides include sequences having at least about 90%, 91%, 92%, 93%, 94%, 95%, 96%, 97%, 98%, 99% or 100% sequence identity to any one of SEQ ID NOS: 8-9. In some embodiments, a linker molecule comprises a linker peptide that is flexible, having no regular secondary structure. Exemplary linker peptides include sequences having at least about 90%, 91%, 92%, 93%, 94%, 95%, 96%, 97%, 98%, 99% or 100% sequence identity to any one of SEQ ID NOS: 10-11. In some embodiments, a first linker comprises a sequence at least about 90%, 91%, 92%, 93%, 94%, 95%, 96%, 97%, 98%, 99% or 100% identical to SEQ ID NO: 12. In some embodiments, a second linker comprises a sequence at least about 90%, 91%, 92%, 93%, 94%, 95%, 96%, 97%, 98%, 99% or 100% identical to SEQ ID NO: 13.

In some embodiments, a GM-CSF molecule comprises a GM-CSF peptide positioned within a scaffold such that the amino-terminus of GM-CSF peptide and the carboxy-terminus of the GM-CSF peptide are each connected to the scaffold. The GM-CSF peptide may be connected at its amino-terminus to the scaffold by a first linker, and connected at its carboxy-terminus to the scaffold by a second linker. As an example, a GM-CSF molecule is provided comprising A1-L1-T-L2-A2, wherein A1 and A2 are the first and second portions of a scaffold A3 (e.g., an antibody sequence), L1 is the first linker, L2 is the second linker, and T is the GM-CSF peptide. A3 may be a heavy or light chain of an antibody variable domain. In some cases, based on the primary sequence of L1, L1 is configured to form a helix. In some cases, based on the primary sequence of L2, L2 is configured to form a helix. In some cases, L1 is configured to form a first helix, L2 is configured to form a second helix, and the first and second helices are configured to form a coiled-coil. The coil-coil may be anti-parallel. In some embodiments, L1 comprises a sequence having no or about 1, 2, 3, or 4 amino acid substitutions or deletions from SEQ ID NO: 8. In some embodiments, L2 comprises a sequence having no or about 1, 2, 3, or 4 amino acid substitutions or deletions from SEQ ID NO: 9. In some embodiments, L1 comprises a flexible linker. L1 may comprise a sequence having no or about 1 or 2 amino acid substitutions or deletions from SEQ ID NO: 10. L1 may comprise a sequence having no or about 1 or 2 amino acid substitutions or deletions from SEQ ID NO: 11. In some embodiments, L2 comprises a flexible linker. L2 may comprise a sequence having no or about 1 or 2 amino acid substitutions or deletions from SEQ ID NO: 10. L2 may comprise a sequence having no or about 1 or 2 amino acid substitutions or deletions from SEQ ID NO: 11. In some embodiments, L1 comprises a sequence having no or about 1, 2, 3, or 4 amino acid substitutions or deletions from SEQ ID NO: 12. In some embodiments, L2 comprises a sequence having no or about 1, 2, 3, or 4 amino acid substitutions or deletions from SEQ ID NO: 13. The L1-T-L2 may comprise a sequence at least about 90%, 91%, 92%, 93%, 94%, 95%, 96%, 97%, 98%, 99% or 100% identical to SEQ ID NO: 18.

Vectors, Host Cells and Recombinant Methods

In one aspect, GM-CSF molecules provided herein comprise a polypeptide sequence. Such GM-CSF molecules disclosed herein (in some cases referred to as GM-CSF protein fusions) may be expressed and purified by known recombinant and protein purification methods. In an exemplary embodiment, a nucleic acid encoding a protein fusion is synthesized, amplified (e.g., by PCR), restriction enzyme digested and gel purified. The digested nucleic acid may be inserted into a replicable vector. The replicable vector containing the digested protein fusion insert may be transformed or transduced into a host cell for further cloning (amplification of the DNA) or for expression. Host cells may be prokaryotic or eukaryotic cells. In addition, phage vectors containing replicon and control sequences that are compatible with the host microorganism may be used as transforming vectors in connection with these hosts. For example, bacteriophage such as λGEM™-11 may be utilized in making a recombinant vector which may be used to transform susceptible host cells such as E. coli LE392. The protein fusions may be expressed intracellularly (e.g., cytoplasm) or extracellularly (e.g., secretion). For extracellular expression, the vector may comprise a secretion signal which enables translocation of the antibody proteins to the outside of the cell.

Suitable host cells for cloning or expression of protein fusion-encoding vectors include prokaryotic and eukaryotic cells. The host cell may be a eukaryotic. Examples of eukaryotic cells include, but are not limited to, Human Embryonic Kidney (HEK) cell (e.g., HEK 293F cell), Chinese Hamster Ovary (CHO) cell, fungi, yeasts, invertebrate cells (e.g., plant cells and insect cells), lymphoid cell (e.g., YO, NSO, Sp20 cell). Other examples of suitable mammalian host cell lines are monkey kidney CV1 line transformed by SV40 (COS-7), baby hamster kidney cells (BHK), mouse sertoli cells, monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor (MMT 060562), TR1 cells, MRC 5 cells, and FS4 cells. The host cell may be a prokaryotic cell (e.g., E. coli).

Host cells may be transformed with vectors containing nucleotides encoding a GM-CSF protein fusion. Transformed host cells may be cultured in media. The media may be supplemented with one or more agents for inducing promoters, selecting transformants, or amplifying or expressing the genes encoding the desired sequences. Methods for transforming host cells are known in the art and may include electroporation, calcium chloride, or polyethylene glycol/DMSO. Host cells may be transfected or transduced with vectors containing nucleotides encoding GM-CSF protein fusions. Transfected or transduced host cells may be cultured in media. The media may be supplemented with one or more agents for inducing promoters, selecting transfected or transduced cells, or expressing genes encoding the desired sequences.

The expressed GM-CSF protein fusions may be secreted into and recovered from the periplasm of the host cells or transported into the culture media. Protein recovery from the periplasm may involve disrupting the host cell. Disruption of the host cell may comprise osmotic shock, sonication and/or lysis. Centrifugation or filtration may be used to remove cell debris or whole cells. The GM-CSF protein fusions may be further purified, for example, by affinity resin chromatography. Alternatively, GM-CSF protein fusions that are secreted into the culture media may be isolated therein. Cells may be removed from the culture and the culture supernatant being filtered and concentrated for further purification of the proteins produced. The expressed polypeptides may be further isolated and identified using commonly known methods such as polyacrylamide gel electrophoresis (PAGE) and Western blot assay.

GM-CSF protein fusion production may be conducted in large quantity by a fermentation process. Various large-scale fed-batch fermentation procedures are available for production of recombinant proteins. Large-scale fermentations have at least 1000 liters of capacity, e.g., about 1,000 to 100,000 liters of capacity. These fermentors use agitator impellers to distribute oxygen and nutrients, especially glucose (a preferred carbon/energy source). Small scale fermentation refers generally to fermentation in a fermentor that is no more than approximately 100 liters in volumetric capacity, and can range from about 1 liter to about 100 liters. In a fermentation process, induction of protein expression is typically initiated after the cells have been grown under suitable conditions to a desired density, e.g., an OD550 of about 180-220, at which stage the cells are in the early stationary phase. A variety of inducers may be used, according to the vector construct employed, as is known in the art and described herein. Cells may be grown for shorter periods prior to induction. Cells are usually induced for about 12-50 hours, although longer or shorter induction time may be used.

To improve the production yield and quality of the GM-CSF protein fusions disclosed herein, various fermentation conditions may be modified. For example, to improve the proper assembly and folding of the secreted GM-CSF protein fusions, additional vectors overexpressing chaperone proteins, such as Dsb proteins (DsbA, DsbB, DsbC, DsbD and or DsbG) or FkpA (a peptidylprolyl cis,trans-isomerase with chaperone activity) may be used to co-transform the host prokaryotic cells. The chaperone proteins have been demonstrated to facilitate the proper folding and solubility of heterologous proteins produced in bacterial host cells.

To minimize proteolysis of expressed heterologous proteins (especially those that are proteolytically sensitive), certain host strains deficient for proteolytic enzymes may be used for the present disclosure. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available.

Standard protein purification methods known in the art may be employed. The following procedures are non-limiting examples of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, hydroxylapatite chromatography, gel electrophoresis, dialysis, and affinity chromatography and gel filtration using, for example, Sephadex G-75.

GM-CSF protein fusions may be concentrated using a commercially available protein concentration filter, for example, an Amicon or Millipore Pellicon® ultrafiltration unit.

Protease inhibitors or protease inhibitor cocktails may be included in any of the foregoing steps to inhibit proteolysis of the GM-CSF protein fusions.

In some cases, a GM-CSF protein fusion may not be biologically active upon isolation. Various methods for “refolding” or converting a polypeptide to its tertiary structure and generating disulfide linkages, may be used to restore biological activity. Such methods include exposing the solubilized polypeptide to a pH usually above 7 and in the presence of a particular concentration of a chaotrope. The selection of chaotrope is similar to the choices used for inclusion body solubilization, but usually the chaotrope is used at a lower concentration and is not necessarily the same as chaotropes used for the solubilization. In most cases the refolding/oxidation solution will also contain a reducing agent or the reducing agent plus its oxidized form in a specific ratio to generate a particular redox potential allowing for disulfide shuffling to occur in the formation of the protein's cysteine bridge(s). Some of the commonly used redox couples include cysteine/cystamine, glutathione (GSH)/dithiobis GSH, cupric chloride, dithiothreitol(DTT)/dithiane DTT, and 2-mercaptoethanol(bME)/di-thio-b(ME). In many instances, a cosolvent may be used to increase the efficiency of the refolding, and common reagents used for this purpose include glycerol, polyethylene glycol of various molecular weights, arginine and the like.

Compositions

Disclosed herein are compositions comprising a GM-CSF peptide, such as a GM-CSF molecule comprising a GM-CSF peptide and a scaffold (e.g., a long-acting GM-CSF molecule). For scaffolds comprising a polypeptide sequence, the GM-CSF molecule in some cases may also be referred to as a GM-CSF protein fusion.

The compositions may further comprise one or more pharmaceutically acceptable salts, excipients or vehicles. Pharmaceutically acceptable salts, excipients, or vehicles for use in the present pharmaceutical compositions include carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants.

Neutral buffered saline or saline mixed with serum albumin are exemplary appropriate carriers. The pharmaceutical compositions may include antioxidants such as ascorbic acid; low molecular weight polypeptides; proteins, such as serum albumin, gelatin, or antibodies; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics, or polyethylene glycol (PEG). Also by way of example, suitable tonicity enhancing agents include alkali metal halides (preferably sodium or potassium chloride), mannitol, sorbitol, and the like. Suitable preservatives include benzalkonium chloride, thimerosal, phenethyl alcohol, methylparaben, propylparaben, chlorhexidine, sorbic acid and the like. Hydrogen peroxide also may be used as preservative. Suitable cosolvents include glycerin, propylene glycol, and PEG. Suitable complexing agents include caffeine, polyvinylpyrrolidone, beta-cyclodextrin or hydroxy-propyl-beta-cyclodextrin. Suitable surfactants or wetting agents include sorbitan esters, polysorbates such as polysorbate 80, tromethamine, lecithin, cholesterol, tyloxapal, and the like. The buffers may be conventional buffers such as acetate, borate, citrate, phosphate, bicarbonate, or Tris-HC1. Acetate buffer may be about pH 4-5.5, and Tris buffer may be about pH 7-8.5. Additional pharmaceutical agents are set forth in Remington's Pharmaceutical Sciences, 18th Edition, A. R. Gennaro, ed., Mack Publishing Company, 1990.

The composition may be in liquid form or in a lyophilized or freeze-dried form and may include one or more lyoprotectants, excipients, surfactants, high molecular weight structural additives and/or bulking agents. In one embodiment, a lyoprotectant is included, which is a non-reducing sugar such as sucrose, lactose or trehalose. The amount of lyoprotectant generally included is such that, upon reconstitution, the resulting formulation will be isotonic, although hypertonic or slightly hypotonic formulations also may be suitable. In addition, the amount of lyoprotectant should be sufficient to prevent an unacceptable amount of degradation and/or aggregation of the protein upon lyophilization. In another embodiment, a surfactant is included, such as for example, nonionic surfactants and ionic surfactants such as polysorbates (e.g., polysorbate 20, polysorbate 80); poloxamers (e.g., poloxamer 188); poly(ethylene glycol) phenyl ethers (e.g., Triton); sodium dodecyl sulfate (SDS); sodium laurel sulfate; sodium octyl glycoside; lauryl-, myristyl-, linoleyl-, or stearyl-sulfobetaine; lauryl-, myristyl-, linoleyl-or stearyl-sarcosine; linoleyl, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl ofeyl-taurate; the MONAQUAT™ series (Mona Industries, Inc., Paterson, N.J.), polyethyl glycol, polypropyl glycol, and copolymers of ethylene and propylene glycol (e.g., Pluronics, PF68 etc). Exemplary amounts of surfactant that may be present in the pre-lyophilized formulation are from about 0.001-0.5%. High molecular weight structural additives (e.g., fillers, binders) may include for example, acacia, albumin, alginic acid, calcium phosphate (dibasic), cellulose, carboxymethylcellulose, carboxymethylcellulose sodium, hydroxyethylcellulose, hydroxypropylcellulose, hydroxypropylmethylcellulose, microcrystalline cellulose, dextran, dextrin, dextrates, sucrose, tylose, pregelatinized starch, calcium sulfate, amylose, glycine, bentonite, maltose, sorbitol, ethylcellulose, disodium hydrogen phosphate, disodium phosphate, disodium pyrosulfite, polyvinyl alcohol, gelatin, glucose, guar gum, liquid glucose, compressible sugar, magnesium aluminum silicate, maltodextrin, polyethylene oxide, polymethacrylates, povidone, sodium alginate, tragacanth microcrystalline cellulose, starch, and zein. Exemplary concentrations of high molecular weight structural additives are from 0.1% to 10% by weight. In other embodiments, a bulking agent (e.g., mannitol, glycine) may be included.

Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers' dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present, such as, for example, anti-microbials, anti-oxidants, chelating agents, inert gases and the like. See generally, Remington's Pharmaceutical Science, 16th Ed., Mack Eds., 1980.

Compositions described herein may be formulated for controlled or sustained delivery in a manner that provides local concentration of the product (e.g., bolus, depot effect) and/or increased stability or half-life in a particular local environment. The compositions may comprise the formulation of GM-CSF molecule proteins, polypeptides, nucleic acids, or vectors disclosed herein with particulate preparations of polymeric compounds such as polylactic acid, polyglycolic acid, etc., as well as agents such as a biodegradable matrix, injectable microspheres, microcapsular particles, microcapsules, bioerodible particles beads, liposomes, and implantable delivery devices that provide for the controlled or sustained release of the active agent which then may be delivered as a depot injection. Techniques for formulating such sustained-or controlled-delivery means are known and a variety of polymers have been developed and used for the controlled release and delivery of drugs. Such polymers are typically biodegradable and biocompatible. Polymer hydrogels, including those formed by complexation of enantiomeric polymer or polypeptide segments, and hydrogels with temperature or pH sensitive properties, may be desirable for providing drug depot effect because of the mild and aqueous conditions involved in trapping bioactive protein agents.

A pharmaceutical composition disclosed herein can be administered to a subject by any suitable administration route, including but not limited to, parenteral (intravenous, subcutaneous, intraperitoneal, intramuscular, intravascular, intrathecal, intravitreal, infusion, or local), topical, oral, and/or nasal administration.

Formulations suitable for intramuscular, subcutaneous, peritumoral, or intravenous injection can include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, an active agent can be optionally formulated in aqueous solutions, preferably in physiologically compatible buffers such as Hank's solution, Ringer's solution, or physiological saline buffer.

Parenteral injections optionally involve bolus injection or continuous infusion. Formulations for injection are optionally presented in unit dosage form, e.g., in ampoules or in multi dose containers, with an added preservative. The pharmaceutical composition described herein can be in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an active agent in water soluble form. Additionally, suspensions are optionally prepared as appropriate oily injection suspensions.

Alternatively or additionally, the compositions may be administered locally via implantation into the affected area of a membrane, sponge, or other appropriate material on to which a GM-CSF molecule disclosed herein has been absorbed or encapsulated. Where an implantation device is used, the device may be implanted into any suitable tissue or organ, and delivery of a GM-CSF peptide, nucleic acid, or vector disclosed herein may be directly through the device via bolus, or via continuous administration, or via catheter using continuous infusion.

A pharmaceutical composition comprising a GM-CSF molecule disclosed herein may be formulated for inhalation, such as for example, as a dry powder. Inhalation solutions also may be formulated in a liquefied propellant for aerosol delivery. In yet another formulation, solutions may be nebulized. For pulmonary delivery, the particle size should be suitable for delivery to the distal lung. For example, the particle size may be from 1 μm to 5 μm; however, larger particles may be used, for example, if each particle is fairly porous.

Certain formulations comprising a GM-CSF molecule disclosed herein may be administered orally. Formulations administered in this fashion may be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents may be included to facilitate absorption of a selective binding agent. Diluents, flavorings, low melting point waxes, vegetable oils, lubricants, suspending agents, tablet disintegrating agents, and binders also may be employed.

Another preparation may involve an effective quantity of a GM-CSF molecule in a mixture with non-toxic excipients which are suitable for the manufacture of tablets. By dissolving the tablets in sterile water, or another appropriate vehicle, solutions may be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

“Pharmaceutically acceptable” may refer to approved or approvable by a regulatory agency of the Federal or a state government or listed in the U.S. Pharmacopeia or other generally recognized pharmacopeia for use in animals, including humans.

“Pharmaceutically acceptable salt” may refer to a salt of a compound that is pharmaceutically acceptable and that possesses the desired pharmacological activity of the parent compound.

“Pharmaceutically acceptable excipient, carrier or adjuvant” may refer to an excipient, carrier or adjuvant that may be administered to a subject, together with at least one antibody of the present disclosure, and which does not destroy the pharmacological activity thereof and is nontoxic when administered in doses sufficient to deliver a therapeutic amount of the compound.

“Pharmaceutically acceptable vehicle” may refer to a diluent, adjuvant, excipient, or carrier with which at least one antibody of the present disclosure is administered.

Therapeutic Use

In one aspect, GM-CSF molecules disclosed herein comprise a GM-CSF peptide for treating, alleviating, inhibiting and/or preventing one or more diseases and/or conditions. In some embodiments, the disease or condition is a neurological disease or condition. Non-limiting examples of neurological diseases or conditions include Parkinson's disease, Alzheimer's disease, or diseases or conditions characterized by neuroinflammation. In some embodiments, the disease or condition is an inflammatory disease or condition. Non-limiting examples of inflammatory diseases or conditions include Crohn's disease and colitis. In some embodiments, the disease or condition is an infectious disease. The infectious disease may comprise a cytomegalovirus infection. In some embodiments, the disease or condition comprises Parkinson's disease (PD). In some embodiments, the disease or condition comprises amyotrophic lateral sclerosis (ALS). In some embodiments, the disease or condition comprises Alzheimer's disease (AD). In some embodiments, the disease or condition comprises traumatic brain injury. In some embodiments, the disease or condition comprises inflammatory bowel disease (IBD) including colitis and Crohn's disease (CD). In some embodiments, the disease or condition comprises acute radiation syndrome. In some embodiments, the disease or condition comprises cancer.

In some embodiments, a GM-CSF molecule disclosed herein is administered following induction chemotherapy in a subject. The GM-CSF molecule may shorten the time to neutrophil recovery and/or reduce the incidence of infections. In some cases, the subject comprises acute myelogenous leukemia.

In some embodiments, a GM-CSF molecule disclosed herein is administered to mobilize hematopoietic progenitor cells into peripheral blood for collection by leukapheresis. Mobilization may allow for the collection of increased numbers of progenitor cells capable of engraftment as compared with collection without mobilization. Following chemotherapy, such as myeloablative chemotherapy, the transplantation of an increased number of progenitor cells can lead to more rapid engraftment.

In some embodiments, a GM-CSF molecule disclosed herein is administered for acceleration of myeloid recovery in a subject having non-Hodgkin's lymphoma (NHL). In some embodiments, a GM-CSF molecule disclosed herein is administered for acceleration of myeloid recovery in a subject having acute lymphoblastic leukemia (ALL). In some embodiments, a GM-CSF molecule disclosed herein is administered for acceleration of myeloid recovery in a subject having Hodgkin's disease. In some embodiments, the subject is undergoing autologous bone marrow transplantation (BMT). GM-CSF administration may accelerate myeloid engraftment as compared to myeloid engraftment without GM-CSF administration. GM-CSF administration may decrease median duration of antibiotic administration as compared to duration without GM-CSF administration. Administration may reduce median duration of infectious episodes as compared to no GM-CSF molecule administration. Administration may shorten median duration of hospitalization as compared to no GM-CSF molecule administration. Hematologic response to GM-CSF can be detected by complete blood court.

In some embodiments, a GM-CSF molecule disclosed herein is administered for acceleration of myeloid recovery in subjects undergoing allogeneic BMT from HLA-matched related donors.

In some embodiments, a GM-CSF molecule disclosed herein is administered to a subject who has undergone allogeneic or autologous bone marrow transplant (BMT) where engraftment is delayed or has failed. Administration may prolong survival of the subject. Recombinant human GM-CSF is used in a variety of hematopoietic disorders, including reducing the severity of chemotherapy-induced neutropenia, accelerating hematopoietic recovery following bone marrow transplantation and mobilizing blood progenitor cells for transplantation. However, recombinant GM-CSF has a short half-life in humans and typically is administered by daily injection for 15-21 days following chemotherapy. The requirement for daily administration also limits the attractiveness of GM-CSF to chemotherapy patients and for the treatment of patients that have been exposed to radiation, such as ARS patients. Accordingly, provided are methods of using a long-acting GM-CSF molecule herein for such conditions, including acute radiation syndrome.

Further, preliminary preclinical results demonstrated that GM-CSF may protect and treat cells from the damages or side effects of radiation toxicity. GM-CSF may help radiation-damaged tissue heal faster.

To assess the efficacy of GM-CSF in humans, retrospective analyses of human patients undergoing cancer treatment and who garner cellular damage following radiation therapy have been performed. It was found that for patients that received GM-CSF prior to radiation therapy, the treatment improved the condition, with some showing damaged tissue healing faster than control groups. Accordingly, methods herein include treating a subject who will undergo radiation therapy, is undergoing radiation therapy, or has undergone radiation therapy, with a GM-CSF molecule herein.

In some embodiments, a GM-CSF molecule disclosed herein is administered for Acute Radiation Syndrome. The GM-CSF molecule may increase survival in subjects exposed to myelosuppressive doses of radiation (Hematopoietic Syndrome of Acute Radiation Syndrome, H-ARS). Myelosuppression occurs when radiation damages the bone marrow. Suppression of the bone marrow blocks the production of blood cells. The GM-CSF molecule may facilitate recovery of bone marrow cells that develop into white blood cells. Therefore the GM-CSF may also be used to treat and/or prevent infection.

GM-CSF plays a critical role in immune modulation and hematopoiesis. Without being bound by therapy, experimental evidence indicates that GM-CSF, which is frequently upregulated in multiple types of human cancers, tags cancer cells to be targeted by the immune system. The activation of GM-CSF receptors promotes the survival, growth and differentiation of many different immune cell types, including neutrophils, macrophages and various T cells, in addition to the direct stimulatory effect on multiple immune functions. Preliminary preclinical results have demonstrated that GM-CSF may stimulate the immune system in different ways to stop or delay tumor cell growth. GM-CSF may increase the number of immune cells found in the bone marrow or peripheral blood. GM-CSF may also induce extensive tumor destruction.

To assess the efficacy of GM-CSF for immuno-oncology, retrospective analyses of human patients diagnosed with cancer was performed. It was found that for who received GM-CSF, the treatment improved their condition. For some patients that received GM-CSF, treatment delayed tumor growth, as compared to control groups. In addition, for some patients that received GM-CSF, tumor size decreased as compared to control groups. These results suggest that GM-CSF can create an advantageous environment for tumor antigen presentation. Accordingly, methods herein include treating a subject having cancer with a GM-CSF molecule herein.

In some embodiments, methods herein include treatment of a subject suffering from dementia, such as Alzheimer's disease, vascular dementia, cerebral amyloid angiopathy (CAA).

Preliminary preclinical results have demonstrated that GM-CSF rapidly reduced cerebral amyloid deposition and completely reversed memory deficits in transgenic mouse models of Alzheimer's Disease (AD). A retrospective analysis was performed on a cognition study of human patients undergoing hematopoietic cell transplantation for cancer and who garnered cognitive impairments from the chemotherapy or irradiation (NCT01409915). In the patients that received a colony-stimulating factor (CSF) to stimulate the bone marrow and recover immune system function, it was found that those who received GM-CSF plus G-CSF significantly improved in cognitive function as compared to those who received G-CSF alone. These findings combined with over two decades of accrued safety data using recombinant human GM-CSF in elderly leukopenic patients support use of GM-CSF as a treatment to reverse cerebral amyloid pathology and cognitive impairment in AD.

Methods of treatment may comprise administering to a subject in need thereof a composition comprising one or more GM-CSF molecules disclosed herein. The composition may further comprise a pharmaceutically acceptable carrier. The GM-CSF molecule may be substantially purified (e.g., substantially free from substances that limit its effect or produce undesired side-effects). The subject may be an animal, including but not limited to animals such as cows, pigs, sheep, goats, rabbits, horses, chickens, cats, dogs, mice, etc. The subject may be a mammal. The subject may be a human. The subject may be a non-human primate. The subject may be a bovine. The subject may be an avian, reptile or amphibian.

Provided herein is a method of treating a disease or condition in a subject in need thereof, the method comprising administering to the subject a composition comprising a GM-CSF molecule comprising a GM-CSF peptide connected to a scaffold comprising an antibody variable domain. In some embodiments, the disease or condition is a neurological disease or condition. In some embodiments, the neurological disease or condition comprises Parkinson's disease. In some embodiments, the disease or condition comprises amyotrophic lateral sclerosis (ALS). In some embodiments, the disease or condition comprises Alzheimer's disease (AD). In some embodiments, the disease or condition comprises traumatic brain injury. In some embodiments, the disease or condition comprises inflammatory bowel disease (IBD) including colitis and/or Crohn's disease (CD). In some embodiments, the disease or condition comprises acute radiation syndrome. In some embodiments, the disease or condition comprises cancer. In some embodiments, the GM-CSF molecule is administered once about every 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, weekly, biweekly, or monthly. The antibody variable domain may comprise a first polypeptide and a second polypeptide, wherein the first or second polypeptide comprise or are otherwise connected to the GM-CSF peptide. In some embodiments, the first polypeptide comprises a sequence at least about 90%, 91%, 92%, 93%, 94%, 95%, 96%, 97%, 98%, 99%, or 100% identical to SEQ ID NO: 6. In some cases, the first polypeptide comprises a sequence at least about 90%, 91%, 92%, 93%, 94%, 95%, 96%, 97%, 98%, 99%, or 100% identical to SEQ ID NO: 7. In some cases, the first polypeptide comprises a sequence at least about 90%, 91%, 92%, 93%, 94%, 95%, 96%, 97%, 98%, 99%, or 100% identical to SEQ ID NO: 5. In some embodiments, the second polypeptide comprises a sequence at least about 90%, 91%, 92%, 93%, 94%, 95%, 96%, 97%, 98%, 99%, or 100% identical to SEQ ID NO: 2. In some cases, the second polypeptide comprises a sequence at least about 90%, 91%, 92%, 93%, 94%, 95%, 96%, 97%, 98%, 99%, or 100% identical to SEQ ID NO: 4. In some cases, the second polypeptide comprises a sequence at least about 90%, 91%, 92%, 93%, 94%, 95%, 96%, 97%, 98%, 99%, or 100% identical to SEQ ID NO: 1. In some embodiments, provided is a GM-CSF molecule comprising an antibody variable domain comprising a light chain sequence comprising a first polypeptide comprising a sequence at least about 90%, 91%, 92%, 93%, 94%, 95%, 96%, 97%, 98%, 99%, or 100% identical to SEQ ID NO: 6, and a heavy chain sequence comprising a second polypeptide comprising a sequence at least about 90%, 91%, 92%, 93%, 94%, 95%, 96%, 97%, 98%, 99%, or 100% identical to SEQ ID NO: 2. In some embodiments, the first polypeptide comprises a sequence at least about 90%, 91%, 92%, 93%, 94%, 95%, 96%, 97%, 98%, 99%, or 100% identical to SEQ ID NO: 31. In some cases, the first polypeptide comprises a sequence at least about 90%, 91%, 92%, 93%, 94%, 95%, 96%, 97%, 98%, 99%, or 100% identical to SEQ ID NO: 30. In some cases, the first polypeptide comprises a sequence at least about 90%, 91%, 92%, 93%, 94%, 95%, 96%, 97%, 98%, 99%, or 100% identical to SEQ ID NO: 7. In some embodiments, the second polypeptide comprises a sequence at least about 90%, 91%, 92%, 93%, 94%, 95%, 96%, 97%, 98%, 99%, or 100% identical to SEQ ID NO: 29. In some cases, the second polypeptide comprises a sequence at least about 90%, 91%, 92%, 93%, 94%, 95%, 96%, 97%, 98%, 99%, or 100% identical to SEQ ID NO: 28. In some cases, the second polypeptide comprises a sequence at least about 90%, 91%, 92%, 93%, 94%, 95%, 96%, 97%, 98%, 99%, or 100% identical to SEQ ID NO: 4. In some embodiments, provided is a GM-CSF molecule comprising an antibody variable domain comprising a light chain sequence comprising a first polypeptide comprising a sequence at least about 90%, 91%, 92%, 93%, 94%, 95%, 96%, 97%, 98%, 99%, or 100% identical to SEQ ID NO: 31, and a heavy chain sequence comprising a second polypeptide comprising a sequence at least about 90%, 91%, 92%, 93%, 94%, 95%, 96%, 97%, 98%, 99%, or 100% identical to SEQ ID NO: 29.

Provided herein is a method of treating a disease or condition in a subject in need thereof, the method comprising administering to the subject a composition comprising a GM-CSF molecule comprising a GM-CSF peptide connected to a scaffold comprising an antibody variable domain comprising a light chain sequence comprising a sequence at least about 90% identical to SEQ ID NO: 26 (DIQMTQSPSTLSASVGDRVTITCKCQLSVGYMHWYQQKPGKAPKLLIYDTSKLASGVPSRFSGSGSG TEFTLTISSLQPDDFATYYCFQGS[X1]PFTFGGGTKLEIKR), wherein the light chain sequence comprises X1 and X1 comprises GM-CSF; and a heavy chain sequence comprising a sequence at least about 90%, 91%, 92%, 93%, 94%, 95%, 96%, 97%, 98%, 99%, or 100% identical to SEQ ID NO: 2. In some embodiments, the disease or condition is a neurological disease or condition. In some embodiments, the neurological disease or condition comprises Parkinson's disease. In some embodiments, the neurological disease or condition comprises amyotrophic lateral sclerosis (ALS). In some embodiments, the disease or condition comprises Alzheimer's disease (AD). In some embodiments, the disease or condition comprises traumatic brain injury. In some embodiments, the disease or condition comprises inflammatory bowel disease (IBD) including colitis and/or Crohn's disease (CD). In some embodiments, the disease or condition comprises acute radiation syndrome. In some embodiments, the disease or condition comprises cancer. In some embodiments, the GM-CSF molecule is administered once about every 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, weekly, biweekly, or monthly. The GM-CSF may be human, bovine or murine GM-CSF. The GM-CSF may comprise a sequence at least about 85%, 86%, 87%, 88%, 89%, 90%, 91%, 92%, 93%, 94%, 95%, 96%, 97%, 98%, 99%, or 100% identical to SEQ ID NO: 16. The GM-CSF may comprise a sequence at least about 85%, 86%, 87%, 88%, 89%, 90%, 91%, 92%, 93%, 94%, 95%, 96%, 97%, 98%, 99%, or 100% identical to SEQ ID NO: 77. The GM-CSF may include a variant or homolog of GM-CSF. In some embodiments, the light chain sequence comprises a sequence at least about 91%, 92%, 93%, 94%, 95%, 96%, 97%, 98%, 99%, or 100% identical to SEQ ID NO: 26. In some embodiments, the light chain sequence comprises a sequence at least about 90%, 91%, 92%, 93%, 94%, 95%, 96%, 97%, 98%, 99%, or 100% identical to SEQ ID NO: 27 (DIQMTQSPSTLSASVGDRVTITCKCQLSVGYMHWYQQKPGKAPKLLIYDTSKLASGVPSRFSGSGSG TEFTLTISSLQPDDFATYYCFQGSGGSGAKLAALKAKLAALKGGGGS[X2]GGGGSELAALEAELAAL EAGGSGPFTFGGGTKLEIKR), wherein the light chain sequence comprises X2 and X2 comprises the GM-CSF. In some embodiments, the heavy chain sequence comprises a sequence at least about 91%, 92%, 93%, 94%, 95%, 96%, 97%, 98%, 99%, or 100% identical to SEQ ID NO: 2. In some embodiments, the light chain comprises a sequence at least about 90%, 91%, 92%, 93%, 94%, 95%, 96%, 97%, 98%, 99%, or 100% identical to SEQ ID NO: 7. In some embodiments, the light chain comprises a sequence at least about 90%, 91%, 92%, 93%, 94%, 95%, 96%, 97%, 98%, 99%, or 100% identical to SEQ ID NO: 5. In some embodiments, the heavy chain comprises a sequence at least about 90%, 91%, 92%, 93%, 94%, 95%, 96%, 97%, 98%, 99%, or 100% identical to SEQ ID NO: 4. In some embodiments, the heavy chain comprises a sequence at least about 90%, 91%, 92%, 93%, 94%, 95%, 96%, 97%, 98%, 99%, or 100% identical to SEQ ID NO: 1. In some embodiments, the GM-CSF molecule further comprises a Fc region comprising reduced effector function as compared to human IgG1 (SEQ ID NO: 25). The reduced effector function may comprise reduced antibody-dependent cellular cytotoxicity (ADCC) and reduced complement dependent cytotoxicity (CDC). The Fc region may comprise a sequence at least about 90%, 91%, 92%, 93%, 94%, 95%, 96%, 97%, 98%, 99%, or 100% identical to SEQ ID NO: 3. In some cases, the Fc region comprises a human IgG1 comprising E233P, L234V, L235A, ΔG236, A327G, A330S, P331S, per Kabat numbering.

Provided herein is a method of treating a disease or condition in a subject in need thereof, the method comprising administering to the subject a composition comprising a GM-CSF molecule comprising a GM-CSF peptide connected to a scaffold comprising an antibody variable domain comprising a heavy chain sequence comprising a sequence at least about 90% identical to SEQ ID NO: 42 (EVQLVESGGGLVQPGGSLRLSCAASGFNIKDTYIHWVRQAPGKGLEWVARITYPTNGYTRYADSVKG RFTISADTSKNTAYLQMNSLRAEDTAVYYCSRWGQGTLVTVSS), wherein the heavy chain sequence comprises X6 and X6 comprises the GM-CSF peptide; and a light chain sequence comprising a sequence at least about 90%, 91%, 92%, 93%, 94%, 95%, 96%, 97%, 98%, 99%, or 100% identical to SEQ ID NO: 31. In some embodiments, the disease or condition is a neurological disease or condition. In some embodiments, the neurological disease or condition comprises Parkinson's disease. In some embodiments, the disease or condition comprises amyotrophic lateral sclerosis (ALS). In some embodiments, the disease or condition comprises Alzheimer's disease (AD). In some embodiments, the disease or condition comprises traumatic brain injury. In some embodiments, the disease or condition comprises inflammatory bowel disease (IBD) including colitis and/or Crohn's disease (CD). In some embodiments, the disease or condition comprises acute radiation syndrome. In some embodiments, the disease or condition comprises cancer. In some embodiments, the GM-CSF molecule is administered once about every 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, weekly, biweekly, or monthly. The GM-CSF may be human, bovine or murine GM-CSF. The GM-CSF may comprise a sequence at least about 85%, 86%, 87%, 88%, 89%, 90%, 91%, 92%, 93%, 94%, 95%, 96%, 97%, 98%, 99%, or 100% identical to SEQ ID NO: 16. The GM-CSF may comprise a sequence at least about 85%, 86%, 87%, 88%, 89%, 90%, 91%, 92%, 93%, 94%, 95%, 96%, 97%, 98%, 99%, or 100% identical to SEQ ID NO: 77. The GM-CSF may include a variant or homolog of GM-CSF. In some embodiments, the heavy chain sequence comprises a sequence at least about 91%, 92%, 93%, 94%, 95%, 96%, 97%, 98%, 99%, or 100% identical to SEQ ID NO: 42. In some embodiments, the heavy chain sequence comprises a sequence at least about 90%, 91%, 92%, 93%, 94%, 95%, 96%, 97%, 98%, 99%, or 100% identical to SEQ ID NO: 43 (EVQLVESGGGLVQPGGSLRLSCAASGFNIKDTYIHWVRQAPGKGLEWVARITYPTNGYTRYADSVKG RFTISADTSKNTAYLQMNSLRAEDTAVYYCSRGGSGAKLAALKAKLAALKGGGGSGGGGSEL AALEAELAALEAGGSGDYWGQGTLVTVSS), wherein the heavy chain sequence comprises X6 and X6 comprises the GM-CSF. In some embodiments, the heavy chain sequence comprises a sequence at least about 91%, 92%, 93%, 94%, 95%, 96%, 97%, 98%, 99%, or 100% identical to SEQ ID NO: 43. In some embodiments, the light chain comprises a sequence at least about 90%, 91%, 92%, 93%, 94%, 95%, 96%, 97%, 98%, 99%, or 100% identical to SEQ ID NO: 7. In some embodiments, the heavy chain comprises a sequence at least about 90%, 91%, 92%, 93%, 94%, 95%, 96%, 97%, 98%, 99%, or 100% identical to SEQ ID NO: 29. In some embodiments, the heavy chain comprises a sequence at least about 90%, 91%, 92%, 93%, 94%, 95%, 96%, 97%, 98%, 99%, or 100% identical to SEQ ID NO: 4. In some embodiments, the heavy chain comprises a sequence at least about 90%, 91%, 92%, 93%, 94%, 95%, 96%, 97%, 98%, 99%, or 100% identical to SEQ ID NO: 28. In some embodiments, the GM-CSF molecule further comprises a Fc region comprising reduced effector function as compared to human IgG1 (SEQ ID NO: 25). The reduced effector function may comprise reduced antibody-dependent cellular cytotoxicity (ADCC) and reduced complement dependent cytotoxicity (CDC). The Fc region may comprise a sequence at least about 90%, 91%, 92%, 93%, 94%, 95%, 96%, 97%, 98%, 99%, or 100% identical to SEQ ID NO: 3. In some cases, the Fc region comprises a human IgG1 comprising E233P, L234V, L235A, ΔG236, A327G, A330S, P331S, per Kabat numbering.

Provided herein is a method of treating a disease or condition in a subject in need thereof, the method comprising administering to the subject a composition comprising a GM-CSF molecule comprising a sequence at least about 90%, 91%, 92%, 93%, 94%, 95%, 96%, 97%, 98%, 99%, or 100% identical to SEQ ID NO: 29, and a second polypeptide comprising a sequence at least about 90%, 91%, 92%, 93%, 94%, 95%, 96%, 97%, 98%, 99%, or 100% identical to SEQ ID NO: 31. In some embodiments, the first polypeptide comprises a sequence at least about 95% identical to SEQ ID NO: 29 and the second polypeptide comprises a sequence at least about 95% identical to SEQ ID NO: 31. In some embodiments, the first polypeptide comprises a sequence at least about 96% identical to SEQ ID NO: 29 and the second polypeptide comprises a sequence at least about 96% identical to SEQ ID NO: 31. In some embodiments, the first polypeptide comprises a sequence at least about 97% identical to SEQ ID NO: 29 and the second polypeptide comprises a sequence at least about 97% identical to SEQ ID NO: 31. In some embodiments, the first polypeptide comprises a sequence at least about 98% identical to SEQ ID NO: 29 and the second polypeptide comprises a sequence at least about 98% identical to SEQ ID NO: 31. In some embodiments, the first polypeptide comprises a sequence at least about 99% identical to SEQ ID NO: 29 and the second polypeptide comprises a sequence at least about 99% identical to SEQ ID NO: 31. In some embodiments, the first polypeptide comprises SEQ ID NO: 29 and a second polypeptide comprises SEQ ID NO: 31. In some embodiments, the disease or condition is a neurological disease or condition. In some embodiments, the neurological disease or condition comprises Parkinson's disease. In some embodiments, the disease or condition comprises amyotrophic lateral sclerosis (ALS). In some embodiments, the disease or condition comprises inflammatory bowel disease (IBD) including colitis and Crohn's disease (CD). In some embodiments, the disease or condition comprises Alzheimer's disease (AD). In some embodiments, the disease or condition comprises traumatic brain injury. In some embodiments, the disease or condition comprises acute radiation syndrome. In some embodiments, the disease or condition comprises cancer. In some embodiments, the GM-CSF molecule is administered once about every 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, weekly, biweekly, or monthly.

Provided herein is a method of treating a disease or condition in a subject in need thereof, the method comprising administering to the subject a composition comprising a GM-CSF molecule comprising a sequence at least about 90%, 91%, 92%, 93%, 94%, 95%, 96%, 97%, 98%, 99%, or 100% identical to SEQ ID NO: 28, and a second polypeptide comprising a sequence at least about 90%, 91%, 92%, 93%, 94%, 95%, 96%, 97%, 98%, 99%, or 100% identical to SEQ ID NO: 30. In some embodiments, the first polypeptide comprises a sequence at least about 95% identical to SEQ ID NO: 28 and the second polypeptide comprises a sequence at least about 95% identical to SEQ ID NO: 30. In some embodiments, the first polypeptide comprises a sequence at least about 96% identical to SEQ ID NO: 28 and the second polypeptide comprises a sequence at least about 96% identical to SEQ ID NO: 30. In some embodiments, the first polypeptide comprises a sequence at least about 97% identical to SEQ ID NO: 28 and the second polypeptide comprises a sequence at least about 97% identical to SEQ ID NO: 30. In some embodiments, the first polypeptide comprises a sequence at least about 98% identical to SEQ ID NO: 28 and the second polypeptide comprises a sequence at least about 98% identical to SEQ ID NO: 30. In some embodiments, the first polypeptide comprises a sequence at least about 99% identical to SEQ ID NO: 28 and the second polypeptide comprises a sequence at least about 99% identical to SEQ ID NO: 30. In some embodiments, the first polypeptide comprises SEQ ID NO: 28 and a second polypeptide comprises SEQ ID NO: 30. In some embodiments, the disease or condition is a neurological disease or condition. In some embodiments, the neurological disease or condition comprises Parkinson's disease. In some embodiments, the disease or condition comprises amyotrophic lateral sclerosis (ALS). In some embodiments, the disease or condition comprises Alzheimer's disease (AD). In some embodiments, the disease or condition comprises traumatic brain injury. In some embodiments, the disease or condition comprises inflammatory bowel disease (IBD) including colitis and/or Crohn's disease (CD). In some embodiments, the disease or condition comprises acute radiation syndrome. In some embodiments, the disease or condition comprises cancer. In some embodiments, the GM-CSF molecule is administered once about every 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, weekly, biweekly, or monthly.

Provided herein is a method of treating a disease or condition in a subject in need thereof, the method comprising administering to the subject a composition comprising a GM-CSF molecule comprising a sequence at least about 90%, 91%, 92%, 93%, 94%, 95%, 96%, 97%, 98%, 99%, or 100% identical to SEQ ID NO: 2, and a second polypeptide comprising a sequence at least about 90%, 91%, 92%, 93%, 94%, 95%, 96%, 97%, 98%, 99%, or 100% identical to SEQ ID NO: 6. In some embodiments, the first polypeptide comprises a sequence at least about 95% identical to SEQ ID NO: 2 and the second polypeptide comprises a sequence at least about 95% identical to SEQ ID NO: 6. In some embodiments, the first polypeptide comprises a sequence at least about 96% identical to SEQ ID NO: 2 and the second polypeptide comprises a sequence at least about 96% identical to SEQ ID NO: 6. In some embodiments, the first polypeptide comprises a sequence at least about 97% identical to SEQ ID NO: 2 and the second polypeptide comprises a sequence at least about 97% identical to SEQ ID NO: 6. In some embodiments, the first polypeptide comprises a sequence at least about 98% identical to SEQ ID NO: 2 and the second polypeptide comprises a sequence at least about 98% identical to SEQ ID NO: 6. In some embodiments, the first polypeptide comprises a sequence at least about 99% identical to SEQ ID NO: 2 and the second polypeptide comprises a sequence at least about 99% identical to SEQ ID NO: 6. In some embodiments, the first polypeptide comprises SEQ ID NO: 2 and a second polypeptide comprises SEQ ID NO: 6. In some embodiments, the neurological disease or condition comprises Parkinson's disease. In some embodiments, the disease or condition comprises amyotrophic lateral sclerosis (ALS). In some embodiments, the disease or condition comprises Alzheimer's disease (AD). In some embodiments, the disease or condition comprises traumatic brain injury. In some embodiments, the disease or condition comprises inflammatory bowel disease (IBD) including colitis and/or Crohn's disease (CD). In some embodiments, the disease or condition comprises acute radiation syndrome. In some embodiments, the disease or condition comprises cancer. In some embodiments, the GM-CSF molecule is administered once about every 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, weekly, biweekly, or monthly.

Provided herein is a method of treating a disease or condition in a subject in need thereof, the method comprising administering to the subject a composition comprising a GM-CSF molecule comprising a sequence at least about 90%, 91%, 92%, 93%, 94%, 95%, 96%, 97%, 98%, 99%, or 100% identical to SEQ ID NO: 1, and a second polypeptide comprising a sequence at least about 90%, 91%, 92%, 93%, 94%, 95%, 96%, 97%, 98%, 99%, or 100% identical to SEQ ID NO: 5. In some embodiments, the first polypeptide comprises a sequence at least about 95% identical to SEQ ID NO: 1 and the second polypeptide comprises a sequence at least about 95% identical to SEQ ID NO: 5. In some embodiments, the first polypeptide comprises a sequence at least about 96% identical to SEQ ID NO: 1 and the second polypeptide comprises a sequence at least about 96% identical to SEQ ID NO: 5. In some embodiments, the first polypeptide comprises a sequence at least about 97% identical to SEQ ID NO: 1 and the second polypeptide comprises a sequence at least about 97% identical to SEQ ID NO: 5. In some embodiments, the first polypeptide comprises a sequence at least about 98% identical to SEQ ID NO: 1 and the second polypeptide comprises a sequence at least about 98% identical to SEQ ID NO: 5. In some embodiments, the first polypeptide comprises a sequence at least about 99% identical to SEQ ID NO: 1 and the second polypeptide comprises a sequence at least about 99% identical to SEQ ID NO: 5. In some embodiments, the first polypeptide comprises SEQ ID NO: 1 and a second polypeptide comprises SEQ ID NO: 5. In some embodiments, the disease or condition is a neurological disease or condition. In some embodiments, the neurological disease or condition comprises Parkinson's disease. In some embodiments, the disease or condition comprises amyotrophic lateral sclerosis (ALS). In some embodiments, the disease or condition comprises Alzheimer's disease (AD). In some embodiments, the disease or condition comprises traumatic brain injury. In some embodiments, the disease or condition comprises inflammatory bowel disease (IBD) including colitis and/or Crohn's disease (CD). In some embodiments, the disease or condition comprises acute radiation syndrome. In some embodiments, the disease or condition comprises cancer. In some embodiments, the GM-CSF molecule is administered once about every 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, weekly, biweekly, or monthly.

Administration

In one aspect, GM-CSF molecules comprising a scaffold provided herein are long-acting, having a half-life greater than the half-life of GM-CSF peptide alone. Such GM-CSF molecules may be administered to a subject in need thereof once every 7 days, once every 8 days, once every 9 days, once every 10 days, once every 11 days, once every 12 days, once every 13 days, once every 14 days, once every 15 days, once every 16 days, once every 17 days, once every 18 days, once every 19 days, once every 20 days, once every 21 days, once every 22 days, once every 23 days, once every 24 days, once every 25 days, once every 26 days, once every 27 days, once every 28 days, once every 29 days, once every 30 days, once every 31 days, once every 32 days, or once a month. A GM-CSF molecule provided herein may be administered about once every two weeks. A GM-CSF molecule provided herein may be administered about once every three weeks. A GM-CSF molecule provided herein may be administered about once every four weeks. A GM-CSF molecule provided herein may be administered about once a month. The amount of the compositions described herein which will be effective in the treatment, inhibition and/or prevention of a disease or disorder may be determined by standard clinical techniques. In addition, in vitro assays may optionally be employed to help identify optimal dosage ranges. The precise dose to be employed in the formulation may also depend on the route of administration, and the seriousness of the disease or disorder, and should be decided according to the judgment of the practitioner and each patient's circumstances. Effective doses may be extrapolated from dose-response curves derived from in vitro, animal model test systems or clinical trials. In non-limiting embodiments, a GM-CSF molecule is administered to treat Parkinson's disease.

Pharmacological Properties

In one aspect, disclosed herein are methods of improving one or more pharmacological properties of GM-CSF. GM-CSF includes at least human, bovine, rat and mouse GM-CSF, and GM-CSF comprising at least about 90% identity to SEQ ID NO: 16 or 77. The method may comprise producing a GM-CSF molecule, such as a GM-CSF molecule comprising a GM-CSF peptide connected to a scaffold as disclosed herein. Examples of pharmacological properties may include, but are not limited to, half-life, stability, solubility, immunogenicity, toxicity, bioavailability, absorption, liberation, distribution, metabolization, and excretion. Liberation may refer to the process of releasing of GM-CSF from the pharmaceutical formulation. Absorption may refer to the process of a substance entering the blood circulation. Distribution may refer to the dispersion or dissemination of substances throughout the fluids and tissues of the body. Metabolization (or biotransformation, or inactivation) may refer to the recognition by an organism that a foreign substance is present and the irreversible transformation of parent compounds into daughter metabolites. Excretion may refer to the removal of the substances from the body.

The half-life of a GM-CSF molecule may be at least about 5 hours to about 1000 hours, at least about 10 hours to about 1000 hours, at least about 15 hours to about 1000 hours, at least about 20 hours to about 1000 hours, at least about 25 hours to about 1000 hours, at least about 30 hours to about 1000 hours, at least about 40 hour to about 1000 hours, at least about 50 hours to about 1000 hours, at least about 60 hours to about 1000 hours, at least about 70 hours to about 1000 hours, at least about 80 hours to about 1000 hours, at least about 90 hours to about 1000 hours, at least about 100 hours to about 1000 hours, at least about 125 hours to about 1000 hours, at least about 150 hours to about 1000 hours, at least about 175 hours to about 1000 hours, at least about 200 hours to about 1000 hours, at least about 225 hours to about 1000 hours, at least about 250 hours to about 1000 hours, at least about 275 hours to about 1000 hours, at least about 300 hours to about 1000 hours, at least about 350 hours to about 1000 hours, at least about 400 hours to about 1000 hours, at least about 450 hours to about 1000 hours, or at least about 500 hours to about 1000 hours. In some embodiments, the half-life of a GM-CSF molecule provided herein is between about 100 hours and about 500 hours, between about 150 hours and about 500 hours, between about 200 hours and about 500 hours, between about 100 hours and about 400 hours, between about 150 hours and about 400 hours, between about 200 hours and about 400 hours, or between about 100 hours and about 300 hours. For instance, the half-life is about 100, 125, 150, 175, 200, 225, 250, 275, or 300 hours. The half-life may be measured in human, rat or mouse blood, serum and/or plasma. The half-life may be measured using a method provided in the examples herein. The half-life may be measured after administration of a GM-CSF molecule to a subject. The half-life may be measured after a GM-CSF molecule is incubated in a biological sample isolated from a subject.

The half-life of a GM-CSF molecule comprising a scaffold may be at least about 2, 5, 10, 20, 30, 40, 50, 60, 70, 80, 90, 100, 120, 140, 160, 180, 200, 225, 250, 275, 300, 325, 350, 375, 400, 425, 450, 475, or 500-fold greater than the half-life of GM-CSF peptide alone (e.g., sargramostim). The half-life of the GM- CSF molecule may be at least about 50-fold greater than the half-life of GM-CSF peptide alone. The half-life of the GM-CSF molecule may be at least about 100-fold greater than the half-life of GM-CSF peptide alone. The half-life of the GM-CSF molecule may be at least about 200-fold greater than the half-life of GM-CSF peptide alone. The half-life of the GM-CSF molecule may be at least about 300-fold greater than the half-life of GM-CSF peptide alone. The half-life of the GM-CSF molecule may be at least about 400-fold greater than the half-life of GM-CSF peptide alone.

Kits

Further disclosed herein are kits which comprise one or more GM-CSF molecules provided herein or compositions thereof. The GM-CSF molecules may be packaged in a manner which facilitates their use to practice methods of the present disclosure. For example, a kit comprises a GM-CSF molecule described herein packaged in a container with a label affixed to the container or a package insert that describes use of the composition in practicing the method. Suitable containers include, for example, bottles, vials, syringes, etc. The containers may be formed from a variety of materials such as glass or plastic. The container may have a sterile access port (for example the container may be an intravenous solution bag or a vial having a stopper pierceable by a hypodermic injection needle). The kit may comprise a container with the GM-CSF molecule contained therein. The kit may further comprise a package insert indicating that the GM-CSF molecule may be used to treat a particular condition. Alternatively, or additionally, the kit may further comprise a second (or third) container comprising a pharmaceutically-acceptable buffer (e.g., bacteriostatic water for injection (BWFI), phosphate-buffered saline, Ringer's solution and dextrose solution). It may further comprise other materials desirable from a commercial and user standpoint, including, but not limited to, other buffers, diluents, filters, needles, and syringes. The GM-CSF molecule may be packaged in a unit dosage form. The kit may further comprise a device suitable for administering the antibody fusion protein according to a specific route of administration. The kit may contain a label that describes use of the GM-CSF molecule.

### EXAMPLES

The activity data provided in the following examples are generally obtained using the molecules defined in the example and exemplified by the provided SEQ ID. It is to be understood that the activities of any molecule disclosed herein may be enhanced or attenuated depending on conditions not relating to the primary sequence, for example, expression and purification conditions.

### Example 1: Generation of Long-Acting GM-CSF

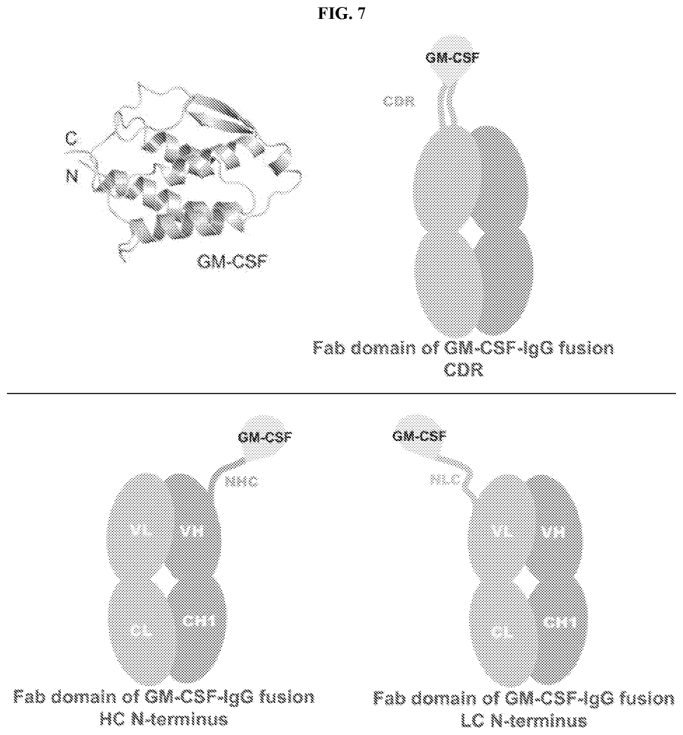

Expression construct: The genes encoding GM-CSF were synthesized by IDT (Coralville, Iowa) and amplified by polymerase chain reaction using PfuUltra II DNA polymerase (Agilent Technologies, CA). The DNA fragments encoding antibody heavy and light chains of palivizumab with reduced RSV-F binding and trastuzumab, along with linkers were also synthesized by IDT and amplified by PCR. The fusion gene fragments were assembled into the pFuse backbone (Invivogen, CA) using Gibson assembly Master Mix (New England Biolabs, MA. The sequences of the resulting mammalian expression vectors were confirmed by DNA sequencing (GENEWIZ, CA). Constructs encoding for the fusions in Table 1 were generated in this manner. FIG. 7 provides schematics of the Fab domains of various long-acting GM-CSF molecules, where GM-CSF is positioned at an amino-terminus or CDR of an IgG scaffold to generate an IgG fusion.

The heavy chain of the long-acting GM-CSF molecules comprise the human IgG1 heavy chain constant region with mutations (E233P, L234V, L235A, ΔG236, A327G, A330S, P331S) to reduce complement-dependent and antibody-dependent cell-mediated cytotoxicities.

Expression and purification: The genes containing the heavy and light chains of each GM-CSF molecule were co-expressed according to Table 1 by transient transfection in HEK 293F cells (Life Technologies, CA). HEK293F cells were cultured in shaker flasks containing FreeStyle medium (Life Technologies, CA), and were shaken at 125 rpm, 37° C., with 5% CO2. For transfection, 293F cells were grown up to a density of one million cells per mL, and were transfected with the light chain, the heavy chain plasmid, and 293fectin at a ratio of 1:2:6 following the manufacturer's instructions. The expression media were harvested at day 5 after transfection to collect the secreted proteins. The fusion antibodies were purified by Protein A chromatography (Thermo Fisher, Ill.) and analyzed by SDS-PAGE and mass spectrometry analysis.

TABLE 1
Long-acting GM-CSF Molecules

| | Heavy Chain | Light Chain | Yield |
|---|---|---|---|
| LA-GMCSF Molecule | (SEQ ID NO) | (SEQ ID NO) | (mg/L) |
| Her-hGMCSF CDR | 28 | 30 | 30 |
| Syn-hGMCSF CDR | 1 | 5 | 11.5-20 |
| Syn-hGMCSF NT (N- | 47 | 48 | 13.8 |
| terminal HC fusion) | | | |
| Syn-hGMCSF NT (N- | 63 | 64 | 12.2 |
| terminal LC fusion) | | | |
| Her-mGMCSF CDR | 55 | 56 | 30 |
| Syn-mGMCSF CDR | 59 | 60 | 12.3 |
| Syn-mGMCSF NT (N- | 51 | 52 | 20.7 |
| terminal HC fusion) | | | |
| hGMCSF-hIgG1 Fc | 67 | — | 3 |
| mGMCSF-hIgG1 Fc | 68 | — | 3 |

Confirmation of Fc null caused by seven-point mutations: To confirm that the seven point mutations in the Fc of protein fusions can significantly reduce the undesired ADCC and CDC effects mediated by Fcγ receptor interactions, an antibody comprising the therapeutic peptide (INVKCSLPQQCIKPCKDAGMRFGKCMNKKCRCYS, SEQ ID NO: 77) positioned within an antibody heavy chain CDR3 having the mutated Fc (SEQ ID NO: 25, Fc null) or wildtype IgG1 Fc labeled with Alexa Fluor 488 were incubated with THP-1, a human monocytic cell line with high expression of Fcγ receptor. The Fc-null antibody fusion did not bind to THP-1 at concentrations up to 100 nM, while the antibody fusion with wildtype IgG1 Fc bound significantly to THP-1 at concentrations as low as 1 nM.

### Example 2: Characterization and in Vitro Activities of Long-Acting GM-CSF Molecules

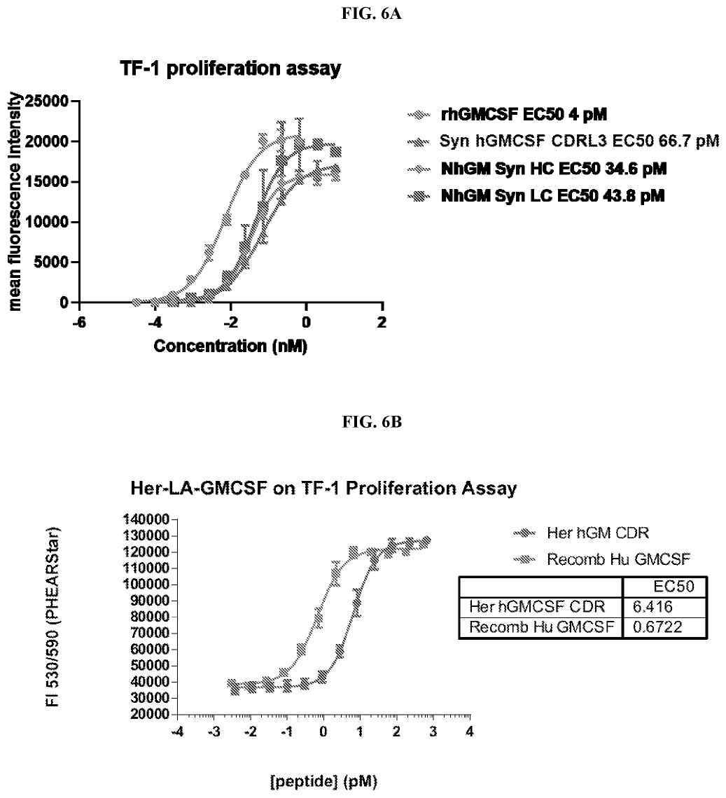

The biological activity of GM-CSF molecules generated in Example 1 in a TF-1 proliferation assay was performed. TF-1 human leukocytes (ATCC, CRL-2003) were cultured in RPM1640 media, ATCC formulation, supplemented with 10% fetal calf serum (FBS) at 37° C. under 5% CO2. For proliferation assay, cells were washed with PBS for 5 times to remove FBS. GM-CSF fusions or commercial GM-CSF standard (R&D systems) were added to cells in 96-well plates at a density of 5,000 cells/well in 150 uL of RPM1640 media with 2% FBS. After 72 hours of incubation, 15uL of AlamarBlue (Invitrogen, DAL1025) was added to each well and fluorescent signal at 565/595 nm was measured after 4 hours of incubation at 37° C. with 5% CO2. Corresponding data is shown in FIGS. 6A-B and Table 2.

TABLE 2
Characterization of Long-acting GM-CSF molecules

| | | | Molecular weight |
|---|---|---|---|
| GM-CSF molecule | EC₅₀ (pM) | Produced by | (Da) |
| Her-hGMCSF CDR | 6.4 | FreeStyle 293 | HC 66508.22 |
| (Her hGMCSF CDR in | (recombinant human | | LC 23443.1 |
| FIG. 6B) | GM-CSF “rhGM- | | |
| | CSF” 0.7) | | |
| Syn-hGMCSF CDR | 60-69 | FreeStyle 293 | HC 49065.6 |
| (Syn hGMCSF CDR3L in | (12.9X as compared to | | LC 41355.7 |
| FIG. 6A) | rhGM-CSF) | | |
| Syn-hGMCSF NT (N- | 34.6 | FreeStyle 293 | HC 64528 |
| terminal HC fusion) | (8.7X as compared to | | LC 23182 |
| (NhGM Syn HC in FIG. | rhGM-CSF) | | |
| 6A) | | | |
| Syn-hGMCSF NT (N- | 43.8 | FreeStyle 293 | HC 49065.6 |
| terminal LC fusion) | (10.9X as compared to | | LC 38644.4 |
| (NhGM Syn LC in FIG. | rhGM-CSF) | | |
| 6A) | | | |
| hGMCSF-hIgG1 Fc | 25 (8.9X as compared | FreeStyle 293 | 41431.82 |
| | to rhGM-CSF) | | |
| Her-mGMCSF CDR | 21.3 (10.X as | FreeStyle 293 | HC 66142.87 |
| | compared to rhGM- | | LC 23443.1 |
| | CSF) | | |
| Syn-mGMCSF CDR | 40.1 (5.9X as | FreeStyle 293 | HC 49065.61 |
| | compared to rmGM- | | LC 40990.34 |
| | CSF) | | |
| Syn-mGMCSF NT (N- | 47.5 (7.0X as | FreeStyle 293 | HC 64162.69 |
| terminal HC fusion) | compared to rmGM- | | LC 23181.98 |
| | CSF) | | |
| Syn-mGMCSF NT (N- | 131 (14.7X as | FreeStyle 293 | HC 49065.61 |
| terminal LC fusion) | compared to rmGM- | | LC 38279.06 |
| | CSF) | | |
| mGMCSF-hIgG1 Fc | 44.7 (6.6X as | FreeStyle 293 | 41066.47 |
| | compared to rmGM- | | |
| | CSF) | | |
| rh GM-CSF | 0.7-1.7 | E. coli-derived | |
| | | recombinant human | |
| | | GM-CSF protein | |
| | | from R&D system | |
| rm GM-CSF | 3.1-15.7 | E. coli-derived | |
| | | recombinant mouse | |
| | | GM-CSF protein | |
| | | from R&D system | |

### Example 3: Pharmacokinetic (PK), Pharmacodynamic (PD), and in Vivo Efficacy of Long-Acting GM-CSF in Rat

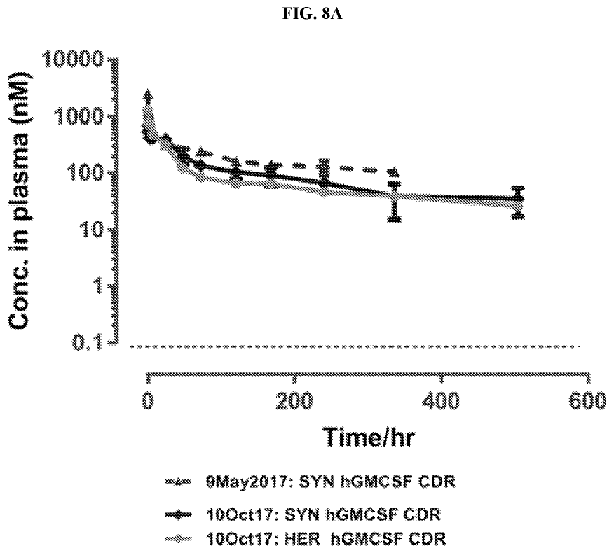

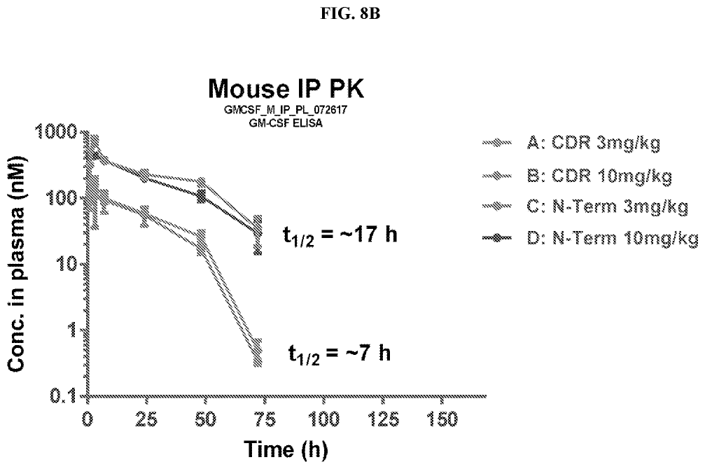

Pharmacokinetic assay in rats: Samples of long-acting GM-CSF proteins were injected intravenously into Sprague-Dawley rats at a single dose of 10 mg/kg, or intraperitoneally into mice at 3 mg/kg or 10 mg/kg. Blood samples were withdrawn at the various time points. The samples were stored in heparinized collection tubes, spun down and stored at −80° C. until further processing. After thawing and proper dilution of sample, the amount of antibody in each blood sample was quantified by ELISA. Data was analyzed by the PK modeling program (WinNonlin, Certara, N.J.) to determine the pharmacokinetic parameters. The experiments were performed using molecules of Table 1, with data presented in FIGS. 8A-B and Table 3. FIG. 8A shows the concentrations of Syn-hGMCSF CDR and Her-hGMCSF CDR in rat plasma over time. FIG. 8B shows the concentrations of Syn-mGMCSF CDR and Syn-mGMCSF NT (N-terminal HC fusion) in mouse plasma over time. Syn-hGMCSF CDR and Her-hGMCSF CDR exhibited half-life extension compared to recombinant GM-CSF (t_{1/2} is about 200 hr vs sargramostim t_{1/2}<0.5 hr). hGMCSF CDR fusion exhibited a Cₘₐₓ of 2,481 nM and AUC∞ (hr*nM) of 63,257.

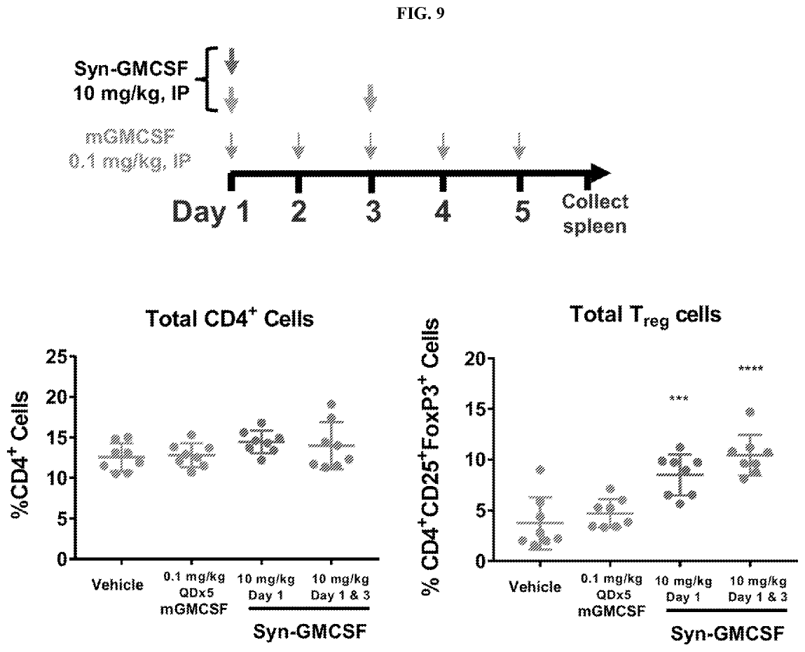

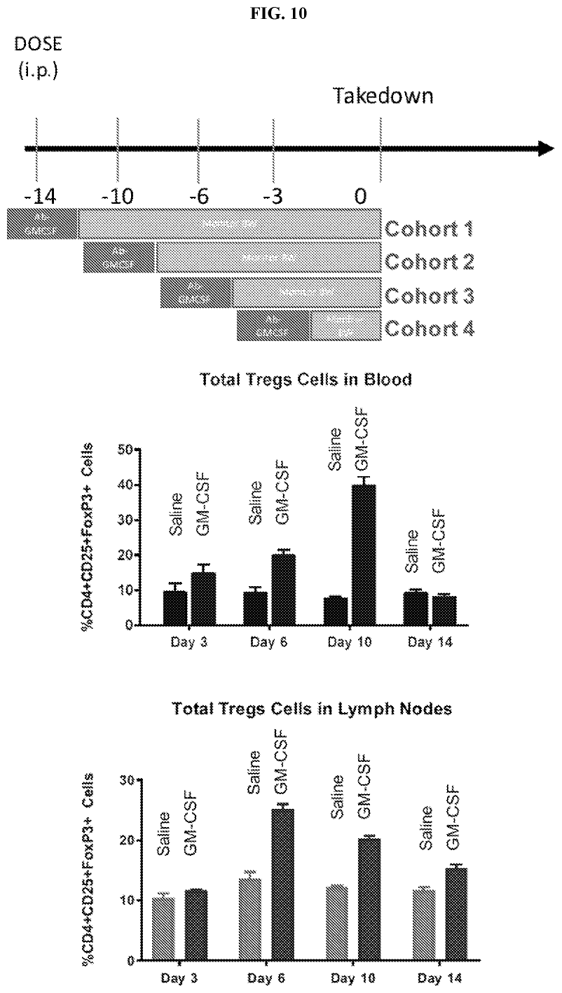

T_{reg} assay: FIG. 9 shows that sub-chronic treatment with Syn-mGMCSF CDR (shown as Syn-GMCSF) significantly increases T_{reg} expansion in mice. FIG. 10 shows that a single long-acting GM-CSF Her-hGMCSF CDR increased T_{reg} expansion for up to 14 days.

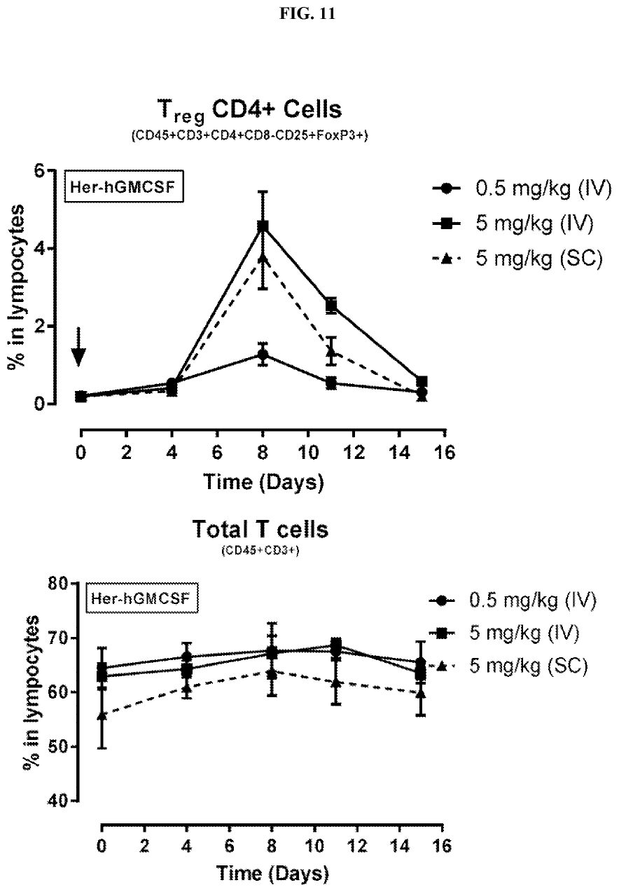

Cynomolgus PK/PD assay: Samples of Her-hGMCSF CDR were injected intravenously (IV) or subcutaneously (SC) into Cynomolgus monkeys (n=4, ˜3 kg, 2-5 years old) at a single dose of 1 mg/kg (IV), 5-10 mg/kg (IV), or 5-10 mg/kg (SC). For plasma PK, 13 time points were collected: pre-dose, 10 min (for SC=30 min), 1, 3, 10, 24, 48, 72, 120, 168, 240, 336, and 504 hours. Blood samples were collected for flow cytometry at 6 time points: pre-dose, 72, 120, 240, 336, and 504 hours. There were no obvious negative clinical signs. As shown in FIG. 11, Her-hGMCSF CDR dose-dependently increases circulating T_{reg}, with peak T_{reg} increase at day 8, and up to 20-fold increase in the 5 mg/kg treated groups.

In vivo efficacy: Assay is described further in Example 4.

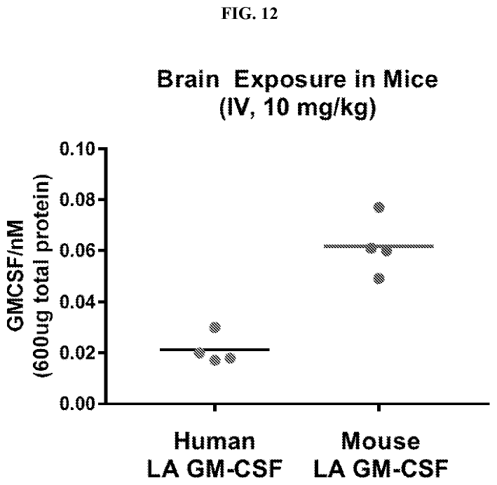

Brain exposure in mice: Long-acting GMCSF (Her-hGMCSF CDR and Her-mGMCSF CDR) is actively transported into the brain of mice as shown in FIG. 12. Samples of long-acting GM-CSF proteins were injected intravenously into CD1 mice (n=4, female, 12 weeks old) at a single dose of 10 mg/kg. Brain tissues were harvested 3 hours post dosing. Brain samples were smashed on dry ice and transferred into homogenization tubes pre-loaded with 1.4 mm ceramic beads. Tissues were homogenized in RIPA buffer supplemented with protease inhibitors. After spinning at 4° C. for 30 mins at 1,7000 g, supernatant was collected and protein concentration was measured. For ELISA assay, plates were coated with 2.5 μg/mL anti-human IgG Fc antibody at 37° C. for 2 hours followed by washing and blocking with 2% nonfat milk in 0.5% PBST for 1 hour. After adding standard protein and diluted samples, plates were incubated at 4° C. for overnight and washed with 0.5% PBST. Then, 1 μg/mL anti human or mouse GMCSF antibody was added to wells, and incubated at room temperature for 2 hours. After washing, 0.5 μg/mL HRP-linked anti-mouse IgG antibody was added followed by 1 hour incubation at room temperature. Pre-warmed TMB substrate was added to wells and incubated in dark for 10 mins at room temperature. Absorbance at 450 nM was measured by the microplate reader.

TABLE 3
Pharmacokinetics, pharmacodynamics, and in vivo efficacy of long-acting GM-CSF

| GMCSF Molecule | Rat PK t_{1/2} (hr) | In vivo PD (T_{reg}) | In vivo efficacy |
|---|---|---|---|
| Her-hGMCSF CDR | ~200 hr | Increased T_{reg} | n/a |
| Syn-hGMCSF CDR | ~200 hr | Cynomolgus req. | n/a |
| Syn-hGMCSF NT (N- | n/a | Cynomolgus req. | n/a |
| terminal HC fusion) | | | |
| Her-mGMCSF CDR | n/a | Increased T_{reg} | Neuroprotection |
| Syn-mGMCSF CDR | 7-17 hr | Increased T_{reg} | Neuroprotection |
| | (see FIG. 8B | | |
| | “CDR”) | | |
| Syn-mGMCSF NT (N- | 7-17 hr | Increased T_{reg} | n/a |
| terminal HC fusion) | (see FIG. 8B “N- | | |
| | Term”) | | |
| rGM-CSF | <0.5 hr | Slightly increased T_{reg} | Neuroprotective in |
| | | | MPTP-Tx mice |

n/a—not evaluated

### Example 4: Neuroprotective and Anti-Inflammatory Capacities of Long-Acting GM-CSF

Parkinson's disease (PD) is characterized by the loss of dopaminergic neurons along the nigrostriatal axis. Immune dysfunction and neuroinflammation are associated with disease progression and neuronal loss. Dysfunctional innate immunity in PD is associated with increased pro-inflammatory cytokine production, microgliosis, reactive oxygen species levels, and neurotoxins, all possessing the capacity to influence neuronal cell death. Likewise, dysfunctions in the adaptive immune response in PD include decreased CD4+ T cell levels, the presence of effector memory T cells, decreased levels of regulatory T cells (T_{reg}), and increased frequencies of Th1 and Th17 T effector T cells (Teff). Without being bound by theory, these immune alterations may contribute to the pathogenesis of disease. Similar adaptive immune aberrations have been observed in amyotrophic lateral sclerosis (ALS), Alzheimer's disease (AD), and Crohn's disease (CD), suggesting a critical role in T_{reg} in suppressing inflammation.

Following treatment with a long-acting GM-CSF in mice, complete blood analyses and blood chemistry profiles were determined by flow cytometric analysis. The neuroprotective and anti-inflammatory capacities of long-acting GM-CSF was assessed in a 1-methyl-4-phenyl-1,2,3,6-tetrahydropyridine (MPTP) model of Parkinson's disease. Treatment with long-acting GM-CSF resulted in T_{reg} induction and enhanced T_{reg} immunosuppressive function

Use of a long-acting GM-CSF composition elicited a neuroprotective immune transformation within the periphery. The observed phenotypic shift and neuroprotective response was greater than observed with recombinant GM-CSF (rGM-CSF) suggesting long-acting GM-CSF as a candidate for the treatment of PD and other neurodegenerative diseases.

Methods

Animals, Antibody GM-CSF Treatment, and MPTP Intoxication

Male, C57BL/6 mice (6-8 weeks old) were obtained from Jackson Laboratories. After acclimation, mice were injected intraperitoneally (i.p.) with a long-acting GM-CSF Her-mGMCSF CDR (SEQ ID NOS: 55, 56). For dose response studies, mice were injected with a single injection at doses ranging from 0 mg/kg-30.0 mg/kg. For rGM-CSF (recombinant granulocyte-macrophage colony-stimulating factor) protein injections, mice were administered either a single injection (1×) or 5 daily injections (5×) prior to either sacrifice or MPTP intoxication at a dose of 0.1 mg/kg. For neuroprotection experiments, mice were injected with either vehicle (DPBS, 10 ml/kg body weight) or 1-methyl-4-phenyl-1,2,3,6-tetrahydropyridine hydrochloride (MPTP-HC1) reconstituted in PBS obtained from Sigma-Aldrich, St. Louis, Mo. Mice received 4 subcutaneous injections of MPTP-HC1 (16 mg/kg, MPTP free base); one injection at 2-hour intervals. MPTP safety precautions were performed in accordance with the MPTP safety and handling protocol. On days 2 and 7 post-MPTP intoxication, mice were sacrificed, and brains were harvested for processing. All animals were housed and maintained in accordance with the National Institutes of Health Institutional guidelines and approved by the Institutional Animal Care and Use Committee (IACUC) of the University of Nebraska Medical Center.

Perfusions and Immunohistochemistry

While under terminal anesthesia (Fatal Plus, pentobarbital), mice were perfused via cardiac puncture with DPBS followed by 4% paraformaldehyde (Sigma-Aldrich) in DPBS. Following perfusion, whole brains were harvested and processed to assess dopaminergic neuron survival in the substantia nigra and the striatum. Frozen midbrain sections were cut to 30 μm, immunostained with anti-tyrosine hydroxylase (TH) (anti-TH, 1:2000, EMD Millipore), and counterstained for Nissl substance. To assess dopaminergic termini, striatal sections were also labeled with anti-TH (1:1000, EMD Millipore). For microglial labeling, midbrain sections were immunostained with anti-macrophage antigen complex-1 (Mac-1) (anti-CD11b, 1:1000, AbD Serotech). To visualize all antibody-labeled tissues, sections were incubated in streptavidin-horseradish peroxidase (HRP) solution (ABC Elite Vector Kit, Vector Laboratories), color was generated using a glucose oxidase color generation system and incubated with diaminobenzidine (DAB) chromogen (ACROS Organics) for visualization. Within the SN, total numbers of Mac-1+cells, TH+Niss1+ (dopaminergic neurons), and TH-Niss1+ (non-dopaminergic neurons) were estimated by stereological analysis using Stereo Investigator software under the optical fractionator module (MBF Bioscience). Densitometry analysis of dopaminergic neuron termini in the striatum was determined using Image J software (National Institutes of Health).

CD4+ T Cell Isolations

Five days after a single dose of Her-mGMCSF CDR or 5 doses of rGM-CSF administration, donor mice were sacrificed and single-cell suspensions were obtained from spleens. For the genomics study, total CD4+ cells were isolated using EasySep Mouse CD4+T Cell Isolation Kit (StemCell) per the manufacturer's instructions. For proliferation assays, CD4+CD25+T_{reg} and CD4+CD25- conventional responder T cells (Tresp) were isolated from spleen using EasySep Mouse CD4+CD25+ Regulatory T Cell Isolation Kit II (StemCell) per the manufacturer's instructions. Isolated cell purity was assessed via flow cytometric analysis and was determined to be >90% for all isolations.

Flow Cytometric Assessments

Five days after treatment, whole blood and spleens were collected to determine T cell and B cell profiles via flow cytometric analysis. Whole blood (50 μl) and splenocytes (1×106) were fluorescently labeled using antibodies against extracellular markers for CD3, CD4, CD25, CD8, CD19, and the intracellular marker for FoxP3. Mouse blood and splenocytes were labeled with PerCP-Cy5.5-anti-CD3 (eBioscience), PE-Cy7-anti-CD4 (eBioscience), PE-anti-CD25 (eBioscience), FITC-anti-CD8 (eBioscience), and PE-anti-CD19 (eBioscience). For intracellular staining, cells were permeabilized for 45 min at 4° C. using FoxP3/Transcription Factor Staining Buffer Set (eBioscience). Cells were then labeled with APC-anti-FoxP3 (eBioscience) followed by fixation. Samples were analyzed with an LSRII flow cytometer and FACSDiva Software (BD Biosciences, San Jose, Calif.). All cell frequencies were determined from the total lymphocyte population.

Blood Chemistry and Peripheral Blood Assessments

At the time of sacrifice, 250 μl whole blood was collected into K2EDTA blood collection tubes for complete blood count (CBC) levels or into heparinized blood collection tubes for blood chemistry and metabolite levels. Following isolation, heparinized blood was centrifuged and plasma was collected. Complete metabolic panels were carried out using VetScan Chemistry Comprehensive Test cartridges (Abaxis) on a VetScan VS2 machine. For CBC analysis, whole blood collected from K2EDTA tubes was immediately assayed on a VetScan HM5 machine.

Statistical Analyses

All values are expressed as mean ±SEM. Differences in between-group means were analyzed using ANOVA followed by Newman-Keuls post hoc test (GraphPad Software, Inc., La Jolla, CA). Comparisons of slope and elevation for CFSE inhibition assays were evaluated using linear regression. Slopes for all lines were significantly non-zero and linear regression analysis values are expressed on corresponding graphs or noted in the figure legends.

Results

Long-acting GM-CSF treatment significantly increases CD4+CD25+FoxP3+ levels in blood and spleen and enhances immunosuppressive function.

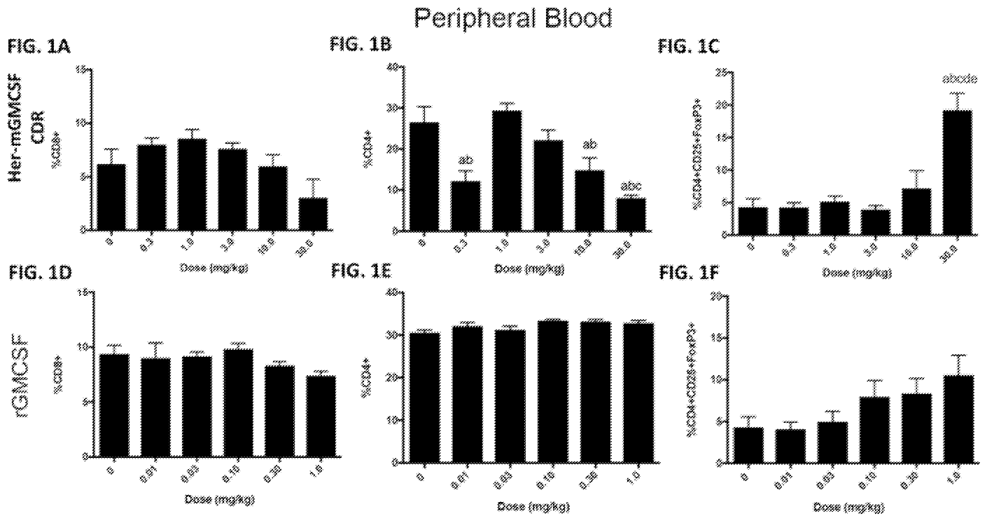

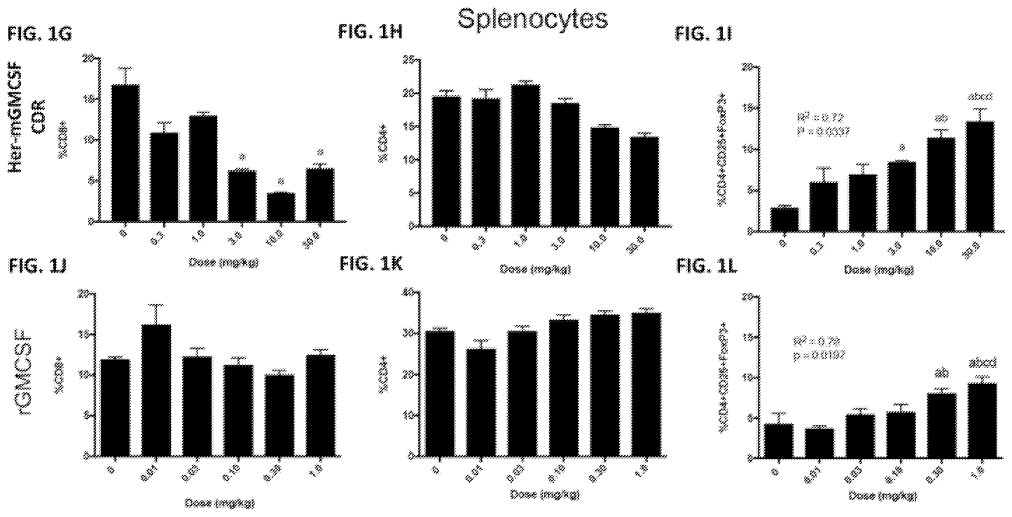

Flow cytometric analysis of lymphocyte populations in peripheral blood revealed that Her-mGMCSF CDR treatment does not affect CD8+ levels (FIG. 1A). However, CD4+ levels were significantly decreased (FIG. 1B). In contrast, only treatment with 30.0 mg/kg Her-mGMCSF CDR significantly increased CD4+CD25+Foxp3+ cell frequency over control levels (FIG. 1C). Treatment with increased dosing of rGM-CSF did not alter CD8+ or CD4+ levels (FIG. 1D and FIG. 1E), but did result in a modest rise in CD4+CD25+FoxP3 levels that was insignificant (FIG. 1F). Flow cytometric analysis of splenic lymphocyte populations following treatment with Her-mGMCSF CDR revealed slightly different results. Her-mGMCSF CDR treatment resulted in a significant decrease in CD8+ levels with 3.0 mg/kg, 10.0 mg/kg, and 30.0 mg/kg treatment (FIG. 1G) and no significant changes in CD4+ levels (FIG. 1H). A dose-dependent increase in CD4+CD25+FoxP3+ levels was also observed (R2=0.72, p=0.0337) (FIG. 1I). Similar to blood observations, rGM-CSF treatment did not alter CD8 or CD4 levels (FIG. 1J and FIG. 1K), but also resulted in a dose-dependent increase in CD4+CD25+FoxP3+ levels (R2=0.78, p=0.0197) (FIG. 1L).

Long-Acting GM-CSF Pretreatment Attenuates Microgliosis and is Neuroprotective in MPTP-Intoxicated Mice.

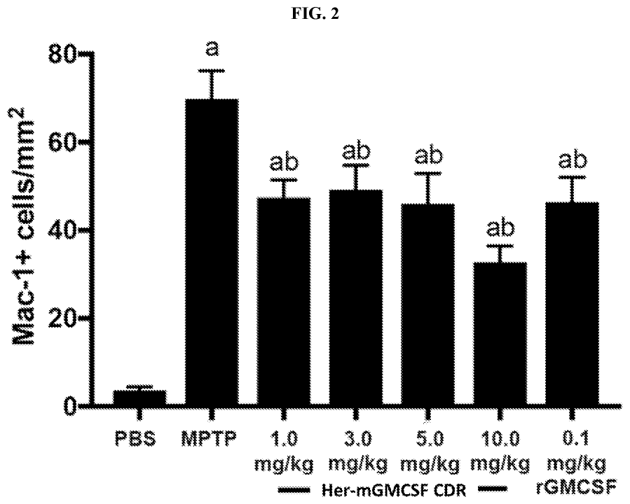

Following T_{reg}-induction assessments, the ability of Her-mGMCSF CDR treatment to attenuate the neuroinflammatory response associated with MPTP intoxication was assessed by quantifying levels of reactive microglia. Two days post-MPTP intoxication, after pretreatment with Her-mGMCSF CDR or rGM-CSF, ventral midbrains were harvested at a time of peak inflammation. Reactivity was assessed by the presence of Mac-1+ microglia with an amoeboid morphology (FIG. 2). MPTP intoxication significantly elevated reactive microglial counts from 4±0.8 cells/mm2 to 70±6.4 cells/mm2 compared to PBS controls (FIG. 2). Treatment with Her-mGMCSF CDR significantly reduced reactive microglia counts to 47±4 cells/mm2 for 1.0 mg/kg, 49±5.5 cells/mm2 for 3.0 mg/kg, 46±6.8 cells/mm2 for 5.0 mg/kg, and 33±3.6 cells/mm2 for 10.0 mg/kg compared to MPTP intoxication alone. Treatment with 0.1 mg/kg rGM-CSF also diminished the neuroinflammatory response by decreasing counts to 46±5.6 cells/mm2.

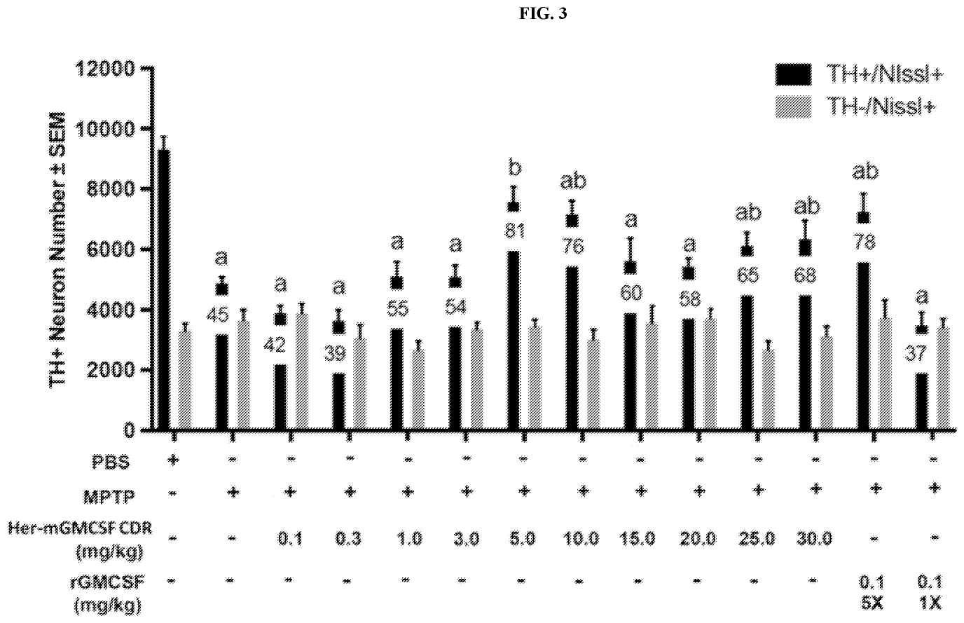

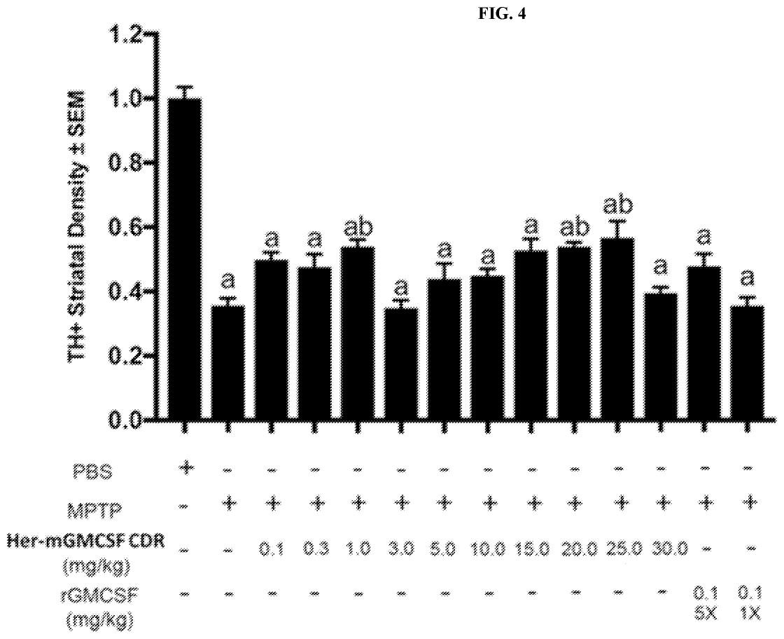

The neuroprotective capacity of Her-mGMCSF CDR pretreatment in the MPTP mouse model was assessed (FIG. 3). Following MPTP intoxication, dopaminergic neuron numbers dropped significantly from 9313±418 to 4873±211 compared to PBS controls (FIG. 3). Pretreatment with a single dose of 0.1 mg/kg, 0.3 mg/kg, 1.0 mg/kg, or 3.0 mg/kg Her-mGMCSF CDR provided no significant neuronal survival, resulting in 3891±248, 3631±364, 5104±488, and 5089±378 neurons after treatment. However, treatment with 5.0 mg/kg and 10.0 mg/kg Her-mGMCSF CDR resulted in significant neuroprotection compared to MPTP intoxication alone, resulting in 81% and 76% neuronal sparing, respectively. In comparison, treatment with 5 consecutive doses of 0.1 mg/kg rGM-CSF also significantly spared dopaminergic neuron numbers, increasing survival from 45% to 78%, respectively. However, use of a single injection of 0.1 mg/kg rGM-CSF did not result in neuroprotection, indicating the requirement for consecutive dosing when utilizing non-long-acting rGM-CSF. Striatal termini survival was also assessed by immunohistochemistry for TH+termini. Digital image analysis indicated that treatment with MPTP significantly diminished striatal termini density compared to PBS-treated controls (FIG. 4). Pretreatment with 1.0 mg/kg, 20.0 mg/kg, and 25.0 mg/kg Her-mGMCSF CDR modestly spared striatal termini reductions; however, levels were still significantly lower than PBS controls. Treatment with all other doses, including Her-mGMCSF CDR and rGM-CSF, did not protect striatal termini, supporting the notion of increased susceptibility of striatal dopaminergic termini.

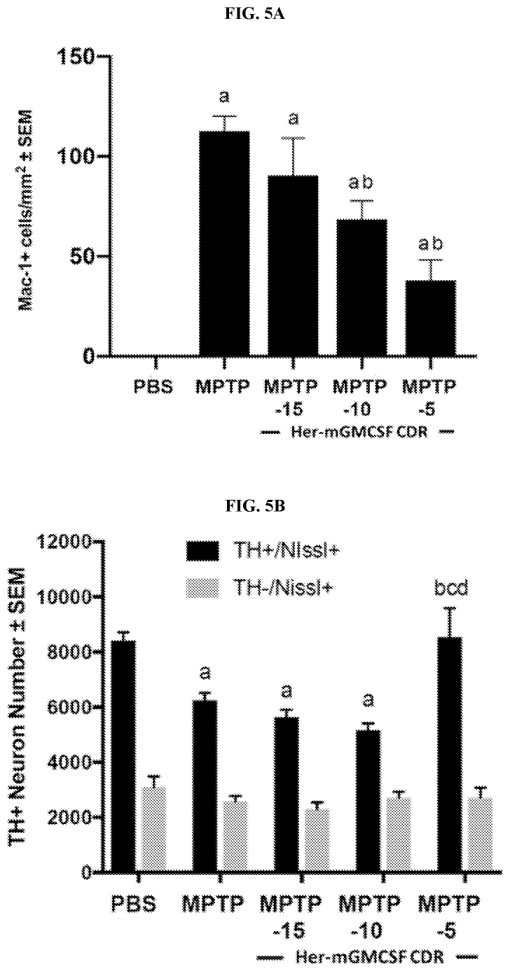

The long-acting therapeutic potential of a single injection of Her-mGMCSF CDR was assessed. Mice were MPTP intoxicated and sacrificed two days or seven days post intoxication to assess the inflammatory response and dopaminergic neuron survival (FIG. 5A and FIG. 5B). MPTP intoxication alone resulted in a significantly increased number of reactive Mac-1+ microglia within the ventral midbrain (FIG. 5A). Treatment with Her-mGMCSF CDR 15 days prior to MPTP intoxication did not attenuate the observed microgliosis. Treating with Her-mGMCSF CDR 10 days or 5 days prior significantly dropped reactive microglial numbers from 113±7 cells/mm2 to 69±9 and 38±10 cells/mm2. Similarly, MPTP intoxication significantly decreased TH+/Niss1+ dopaminergic neuron counts from 8418±130 to 6252±292 (FIG. 5B). However, only Her-mGMCSF CDR treatment 5 days prior to MPTP intoxication resulted in significant neuronal sparing, bringing neuron counts back to control levels. Taken together, these findings indicate a potential Her-mGMCSF CDR therapeutic treatment duration up to 10 days.

These experiments show that peripheral Her-mGMCSF CDR treatment increased T_{reg} frequency and activity, attenuated the neuroinflammatory response, and selectively mitigated MPTP-induced neurotoxicity.

Long-Acting GM-CSF Treatment Results in an Anti-Inflammatory CD4+ T Cell Phenotype.

The effect of treatment with 10 mg/kg Her-mGMCSF CDR on peripheral adaptive immune populations, specifically CD4+ T cells was examined. Transcriptomic analysis of CD4+ T cells after treatment with Her-mGMCSF CDR resulted in significant dysregulation of numerous genes associated with T cell differentiation normalized to PBS-treated controls. In comparison, treatment with 0.1 mg/kg rGM-CSF yielded only minor fold changes in gene expression when normalized to PBS controls

Use of Long-Acting GM-CSF for Neuroinflammatory Diseases

GM-CSF treatment has the potential to modify neuroinflammatory diseases based on its known anti-inflammatory capacity and ability to induce T_{reg} populations. However, an obstacle for clinical translation is its relatively short half-life and limited bioavailability. The examples presented here characterizes the neuroprotective potential of a long-acting GM-CSF formulation, Her-mGMCSF CDR.

Treatment with Her-mGMCSF CDR resulted in a dose-dependent increase in T_{reg} numbers that could be sustained up to 10 days after a single injection and also resulted in increased cellular function within the peripheral blood and spleen above those observed with rGM-CSF treatment. Elevations in T_{reg} numbers following a single injection are also extended for up to 14 days post injection, supporting its long-acting immune-modulating potential. Likewise, T_{reg} isolated from Her-mGMCSF CDR-treated mice displayed increased anti-proliferative effects and were able to suppress Tresp proliferation to a greater extent than T_{reg} isolated from rGM-CSF-treated animals.

Daily dosing of rGM-CSF has the potential to increase neuronal survival and decrease microgliosis in models of neurodegenerative disease. With the long-acting nature of Her-mGMCSF CDR, nearly identical levels of neuroprotection in the MPTP mouse model were achieved using a single injection scheme. Treatment with Her-mGMCSF CDR significantly spared dopaminergic neuronal cell bodies along with their projections into the striatum in MPTP-lesioned mice. Her-mGMCSF CDR treatment also resulted in a larger anti-inflammatory response than rGM-CSF treatment alone as indicated by decreased microgliosis within the lesion site. The resulting decrease in reactive microglial populations may be due to the overall anti-inflammatory phenotype shift observed in the overall CD4+ T cell population following Her-mGMCSF CDR treatment. MPTP intoxication generally results in about 10% neuronal death from the neurotoxin itself. However, for an MPTP-induced lesion to progress and manifest, CD4+ T cells are required to maintain the inflammatory microenvironment within the brain. Following MPTP intoxication, CD4+ T cells cross the blood-brain barrier (BBB), interact with microglia, shift their phenotype, and enhance production of proinflammatory and neurotoxic mediators.

The experiments performed in the examples demonstrate that long-acting GM-CSF molecules described herein may provide decreased dosing frequency and increased GM-CSF bioavailability. Her-mGMCSF CDR treatment selectively induced T_{reg} populations more than 2-fold and enhanced T_{reg} immunosuppressive and anti-proliferative cellular functions. Her-mGMCSF CDR treatment spared dopaminergic neurons within the SN following MPTP intoxication and decreased the corresponding immune-mediated inflammatory response. The decrease in microgliosis and resulting neuroprotection was linked to the anti-inflammatory and regulatory T cell phenotype afforded by Her-mGMCSF CDR treatment. Along with its robust neuroprotective and anti-inflammatory profile, all resulting positive changes were achieved using a single dose of Her-mGMCSF CDR, rather than five consecutive doses, supporting the notion that long-acting GM-CSF treatment is translationally appealing and clinically beneficial for the treatment of PD.

List of abbreviations: AD—Alzheimer's disease, ALS—amyotrophic lateral sclerosis, CFSE-5(6)-carboxyfluorescein N-hydroxysuccinimidyl ester, DAB-3,3′-diaminobenzidine, DPBS-Dulbecco's phosphate-buffered saline, GM-CSF—granulocyte-macrophage colony-stimulating factor, HRP—horse radish peroxidase, IPA—ingenuity pathway analysis, MPTP—1-methyl-4-phenyl-1,2,3,6-tetrahydropyridine, PBS—phosphate-buffered saline, PD—Parkinson's disease , rGM-CSF—recombinant granulocyte-macrophage colony-stimulating factor, RT-PCR—reverse transcription polymerase chain reaction, SEM—standard error of the mean, TBI—traumatic brain injury, TH—tyrosine hydroxylase, UPDRS—Unified Parkinson's Disease Rating Scale.

### Example 5: Parkinson's Disease Clinical Trial

A randomized, double-blind, placebo-controlled, single/multiple ascending dose study of a long-acting GM-CSF from Table 1 (e.g., Her-hGMCSF CDR, Syn-hGMCSF CDR) is performed on healthy subjects and subjects with Parkinson's disease.

Inclusion Criteria:

Indication: Idiopathic Parkinson's disease (moderate idiopathic PD—Hoehn and Yahr 1-3 with bradykinesia plus another PD sign (resting tremor or rigidity), receiving PD medications vs. washout.

Healthy subjects vs. Parkinson's disease patients: Ph1a: healthy->Ph1b PD patients or Ph1a/b: PD patients.

Number of subjects per group (10), 6 ascending-dose cohorts and randomly assigned to receive long-acting GM-CSF or placebo.

Duration of treatment (8 week run-in, 8-26 weeks of treatment), administration every 2-4 weeks of long-acting GM-CSF or placebo and monitored during observational period.

Exclusion Criteria: abnormal MRI, significant lab abnormalities.

End Points:

Safety and tolerability assessment: physical and neurological examinations, laboratory tests, vital signs, and adverse events.

Pharmacokinetic parameters (serum and CSF): maximum long-acting GM-CSF concentration, area under the curve, and half-life.

Pharmacodynamics: T_{reg}, WBC, motor control and mobility improvements, UPDRS scores.

Immunogenicity (ADA).

### Example 6: Bone Marrow Transplant Clinical Trial

Subjects are treated with placebo or GM-CSF molecule exemplified herein after autologous or allogeneic bone marrow or Peripheral Blood Progenitor Cell (PBPC) transplantation. Pediatric and adult patients receive bi-weekly intravenous infusions of the GM-CSF molecule or placebo for 30 days.

### Example 7: Treatment of ALS with Long-Acting GM-CSF in Mouse Model

Previous experiments have demonstrated that SOD1 mouse models of ALS are predictive of the success of therapy in humans (Cleveland and Rothstein (2001), Nat Rev Neurosci, 2, 806-19). Primary endpoints in such analyses are both onset of motor signs, and mortality. For example, the onset of motor signs can be defined as the first day that a mouse cannot remain on the rotarod for 7 min at a speed of 20 rpm (Li, et al (2000), Science, 288, 335-9). Mortality is scored as the day of death, or the day where deficits are so severe that the mouse has to be sacrificed (e.g., apathy and inability to right itself). Additional parameters are determined by the measurement of motor strength by grip strength tests, counts of motor neurons in the spinal cord, nerve thickness (e.g., sciatic nerve, phrenic nerve), and the presence of apoptotic stainings in spinal cord motor neurons. GM-CSF molecules of Table 1 are infused via an osmotic pump into the cerebral ventricles at a pre-determined dose, e.g., at 60 ug/kg body weight/day. Alternatively, GM-CSF molecules are given via i.v. or i.p. injection at a dose of 60 ug/kg body weight per day, or higher doses. Treatment is started at day 60 in the late presymptomatic stage of the SOD1 G93A mutant. In nontreated familial ALS mice, motor impairments appear at 12-14 weeks of age, whereas paralysis is not observed before 20 weeks of age. Life expectancy is 140-170 days. Mice are monitored for effective treatment, including prolonged life as compared to the control group by more than 15% (Cleveland and Rothstein (2001), Nat. Rev. Neurosci., 2, 806-19). As a control group for treatment, both vehicle and zVADfmk (a potent caspase inhibitor that has shown efficacy in this model) treated animals are used. Each group comprises 10 animals each.

### Example 8: Long-acting GM-CSF for the Treatment of ALS

ALS patients are treated with a GM-CSF molecule of Table 1 and monitored for improvement in motor function.

### Example 9: Treatment of Acute Radiation Syndrome with Long-Acting GM-CSF

GM-CSF molecules of Table 1 are administered to mice after irradiation to demonstrate efficacy in increasing survival after radiation exposure.

Briefly, mus musculus/C57BL/6 mice are used for these studies. An LD50/30 is the dose of radiation expected to cause death to 50% of an exposed population within 30 days. An LD 70/30 is the dose of radiation expected to cause death to 70% of an exposed population within 30 days. A dose of radiation equal to the LD50/30 or LD70/30 is delivered as a single uniform total body dose of gamma radiation. The efficacy of the GM-CSF molecules or control (vehicle) to increase 30 day survival is tested at different doses of GM-CSF and different doses of radiation. Mice are monitored for survival until day 30. Endpoints for the study include 30 day overall survival, mean survival time (MST) and Complete Blood Count (CBC) analyses. Increased CBC may indicate accelerated hempatopoietic recovery in GM-CSF treated mice compared to control mice not treated with GM-CSF. This experiment is repeated to determine whether administration of GM-CSF molecules prior to radiation exposure will improve survival.

Experiments similar to those described here are performed to determine whether administration of the molecules of Table 1 improves survival and accelerates hematopoietic recovery in other animal species such as dogs and monkeys following lethal irradiation. Radiation doses can be determined that correspond to LD50 to LD70 doses. Survival can be followed for 30-60 days following irradiation. The dosing frequency and/or dose of the proteins can be adjusted to reflect the differences in clearance rates of the molecules between species. The optimum dose and dosing regimen can be determined for each molecule.

### Example 10: Treatment of Radiation Toxicity with Long-Acting GM-CSF

Patients undergoing radiation therapy are treated with a GM-CSF molecule of Table 1 before radiation therapy, during radiation therapy, or after radiation therapy. Patients are monitored for reduction of cellular damage following the radiation therapy.

### Example 10: Long-Acting GM-CSF for Treatment of Cancer

Patients diagnosed with cancer are treated with a GM-CSF molecule of Table 1. Patients are monitored for delay of tumor growth and tumor size decrease.

### Example 11: Treatment of Alzheimer's Disease with Long-Acting GM-CSF in Mouse Model

Mice are treated with a GM-CSF molecule of Table 1. It is determined if untreated mice show impairment compared to treatment groups.

Radial Arm Water Maze (RAWM) Testing to Evaluate Short-Term Memory

The experiment is performed as generally described (Arendash et al. (2001); Ethell et al. (2006); Arendash et al. (2007)).

Briefly, an aluminum insert is placed into a circular pool to create 6 radially distributed swim arms emanating from a central circular swim area. An assortment of 2-D and 3-D visual cues surround the pool. The number of errors prior to locating which one of the 6 swim arms contain a submerged escape platform is determined for 5 trials/day over 8 days of pre-treatment testing and 4 days of post-treatment testing. During each trial (60 s maximum), the mouse is returned to that trial's start arm upon swimming into an incorrect arm and the number of seconds required to locate the submerged platform is recorded. If the mouse does not find the platform within a 60-s trial, it is guided to the platform for the 30-s stay. The numbers of errors and escape latency are both considered indices of working memory and are temporally similar to the standard registration/recall testing of specific items used clinically in evaluating AD patients.

Cognitive Interference Task

The experiment is performed as described (Loewenstein et al. (2004).

Following post-treatment completion of RAWM testing (4 days), all mice are further evaluated in a novel cognitive interference task for 6 days. This task involves two radial arm water maze set-ups in two different rooms, and 3 different sets of visual cues. The task requires animals to remember a set of visual cues, so that following interference with a different set of cues, the initial set of cues can be recalled to successfully solve the radial arm water maze task.

### Example 12: Treatment of Alzheimer's Disease with Long-Acting GM-CSF

Clinical Trial to assess the safety and efficacy of GM-CSF molecules of Table 1 in treating patients with mild cognitive impairment due to AD.

A GM-CSF molecule of Table 1 or placebo is administered to subjects up to 24 weeks. Florbetapir is administered for PET scans to examine baseline brain imaging pathology and changes on treatment.

Primary outcome measures: Change from baseline in standardized uptake value ratio as measured by PET using florbetapir F18 (Amyvid) [Time Frame: From baseline to Week 24]

Secondary outcome measures: Number of patients experiencing treatment-emergent adverse events (TEAEs) [Time Frame: Week 24]; Change from baseline in CSF analysis [Time Frame: Prior to first injection on Day 1 to serve as a baseline for any necessary follow-up, and optional assessment at Day 155]; MRI to assess for emergence of amyloid related imaging abnormalities (ARIA) [Time Frame: At Screening and Days 43, 85, and 155]; Measurement of antidrug antibody levels [Time Frame: At Days 1, 29, 57, 85, and 155]

Inclusion Criteria:

Men and women ≥40 years and ≤80 years with a diagnosis of MCI due to AD according to the National Institutes of Aging Alzheimer's Association (NIA-AA) criteria (intermediate or high likelihood) with sporadic or familial inheritance pattern. Mild cognitive impairment AD is defined as: evidence of concern about change in cognition, in comparison with person's previous level (subjective memory complaint/decline during the past year for more than 6 months and/or confirmed by informant and/or clinician), and objective impairment of memory function documented by an error score on the delayed recall section of the Alzheimer's Disease Assessment Scale-cognitive subscale (ADAS-cog) ≥1.5 standard deviations (SD) from the age-stratified mean; i.e., Age 55-69 years: ≥6 errors, Age 70-74 years: ≥7 errors, Age 75+ years: ≥8 errors; but no definite impairment(s) in activities of daily living (ADLs), in the Investigator's view as assessed by the Alzheimer's Disease Cooperative Study (ADCS) ADL adapted to MCI, and evidence of elevated cortical amyloid by positron emission tomography (PET) using florbetapir F18 (Amyvid) (a positive scan) by qualitative assessment according to the product label.

Have a dedicated partner/caregiver informant who can assist the patient with the study procedures and administration of study medication, and is in the company of the patient at least 12 hours a week.

Willing and able to provide signed informed consent.

Exclusion criteria:

Prior treatment with an investigational anti-amyloid therapy.

Contraindication for lumbar puncture, or contraindication or inability to complete magnetic resonance imaging (MRI) or having past or planned exposure to ionizing radiation that would, together with the radiation resulting from the administrations of the PET tracer used in this study, exceed applicable institutional, local, or national recommendations for annual or lifetime exposure.

Modified Hachinski Ischemic Score≥4.

Other neurological or psychiatric condition (other than AD) which can impair cognition; or, computerized tomography (CT)/MRI evidence of potentially significant intracranial abnormalities not related to AD (e.g., evidence of major stroke or lacune in an area critical to cognition, infections, cancer, hydrocephalus, multiple sclerosis etc.); or abnormal cerebrospinal fluid (CSF) not consistent with AD.

MRI evidence of >4 microhemorrhages: patients who may be prone to spontaneous amyloid-related imaging abnormalities (ARIA-H) and/or may be more susceptible to adverse effects of the ARIA-H.

Untreated or unstable medical condition that could interfere with the study assessments in the opinion of the Investigator or may require immune-stimulating, immune-suppressive, or immune-modulating treatment(s) during the conduct of the study; e.g., immunoglobulin, therapeutic vaccines, cytokines, anti-cytokine monoclonal antibodies. History of asplenia, hyposplenia, or splenectomy (whatever the surgical reasons).

Current mood or anxiety disorder, and/or a psychotic disorder, and/or a substance-related disorder according to Diagnostic and Statistical Manual of Psychiatric Disorders, Edition W, text revision (DSM-IV-TR) or DSM-V; or considered suicidal or shows suicidal ideation as assessed by the Investigator.

Laboratory abnormalities indicative of an untreated medical or hematologic condition that could increase risk or interfere with study assessments including untreated hypo- or hyperthyroidism, vitamin B12 deficiency, hyperleukocytic syndrome (including but not restricted to chronic myelogenous leukemia, Hodgkin and non-Hodgkin lymphoma), monoclonal gammopathy, and thrombocythemia.

Known renal dysfunction or serum creatinine>150 μmol/L.

Known hepatic dysfunction (apart from Gilbert's syndrome) or serum alanine aminotransferase (ALT) ≥3 times the upper limit of normal (ULN).

Pregnant or breastfeeding woman.

Presence or history of drug hypersensitivity; or known hypersensitivity to sargramostim, yeast-derived products, any other component of the product, or benzyl alcohol (present in bacteriostatic water or saline for infusion).

Evidence of fluid retention (clinical or radiological), respiratory symptoms (e.g., dyspnea), cardiovascular symptoms or electrocardiographic evidence of cardiac disease which warrant therapeutic intervention (e.g., supraventricular arrhythmia).

History of deep vein thrombosis (DVT) or pulmonary embolism or familial predisposition for DVT or pulmonary embolism.

Women and female partners of childbearing potential and not protected by highly effective contraceptive methods of birth control (i.e., oral or depot contraceptives or intrauterine device [IUD] or subject was surgically sterilized) and/or unwilling or unable to be tested for pregnancy; or are pregnant or lactating.

Recipient of an investigational drug within prior 60 days, or within 5 times the elimination half-life of that drug, whichever is the longest.

History of latex allergy or yeast allergy.

Any patient who:

Is likely to be noncompliant, leave the area, or separate from the designated caregiver/informant for more than 3 days during the study,

Unable to cooperate because of a language problem or poor mental development, or

Oversees or implements any aspect of the study.

The preceding merely illustrates the principles of this disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of this disclosure and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of the present disclosure is embodied by the appended claims.

TABLE 4
Antibody Sequences

| Name | SEQ ID NO | Sequence |
|---|---|---|
| GMCSF CDR3L | 1 | QVTLRESGPALVKPTQTLTLTCTFSGFSLSTSGMSVGWIROPPG |
| HC amino acid | | KALEWLADIWWDDKKDYNPSLKSRLTISKDTSKNQVVLKVTN |
| | | 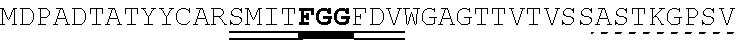 |
| | | 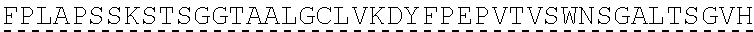 |
| | | 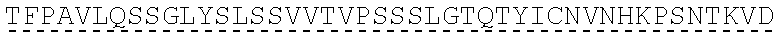 |
| | | 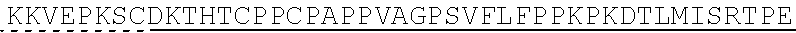 |
| | | VTCVVVDVSHEDPEVKFNWYVDGVEVHNAKTKPREEQYNST |
| | | YRVVSVLTVLHQDWLNGKEYKCKVSNKGLPSSIEKTISKAKGQ |
| | | PREPQVYTLPPSRDELTKNQVSLTCLVKGFYPSDIAVEWESNGQ |
| | | PENNYKTTPPVLDSDGSFFLYSKLTVDKSRWQQGNVFSCSVMH |
| | | EALHNHYTQKSLSLSPGK |
| GMCSF CDR3L | 2 | QVTLRESGPALVKPTQTLTLTCTFSGFSLSTSGMSVGWIRQPPG |
| VH amino acid | | KALEWLADIWWDDKKDYNPSLKSRLTISKDTSKNQVVLKVTN |
| | | MDPADTATYYCARSMITFGGFDVWGAGTTVTVSS |
| GMCSF | 3 | DKTHTCPPCPAPPVAGPSVFLFPPKPKDTLMISRTPEVTCVVVD |
| CDR3L/CDR3H | | VSHEDPEVKFNWYVDGVEVHNAKTKPREEQYNSTYRVVSVLT |
| Fc amino acid | | VLHQDWLNGKEYKCKVSNKGLPSSIEKTISKAKGQPREPQVYT |
| | | LPPSRDELTKNQVSLTCLVKGFYPSDIAVEWESNGOPENNYKT |
| | | TPPVLDSDGSFFLYSKLTVDKSRWQQGNVFSCSVMHEALHNH |
| | | YTQKSLSLSPGK |
| GMCSF | 4 | 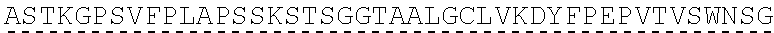 |
| CDR3L/CDR3H | | 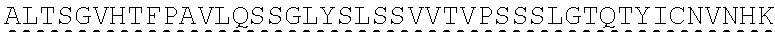 |
| CHI amino acid | | 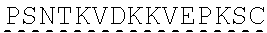 |
| GMCSF CDR3L | 5 | DIQMTQSPSTLSASVGDRVTITCKCQLSVGYMHWYOQKPGKA |
| LC amino acid | | PKLLIYDTSKLASGVPSRFSGSGSGTEFTLTISSLOPDDFATYYCF |
| | | 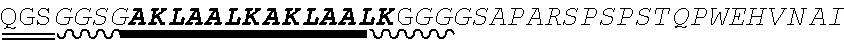 |
| | | QEARRLLNLSRDTAAEMNETVEVISEMFDLQEPTCLQTRLELYKQG |
| | | LRGSLTKLKGPLTMMASHYKQHCPPTPETSCATQIITFESFKENLK |
| | | 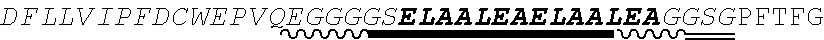 |
| | | GGTKLEIKRTVAAPSVFIFPPSDEOLKSGTASVVCLLNNFYPREA |
| | | KVQWKVDNALQSGNSQESVTEQDSKDSTYSLSSTLTLSKADYE |
| | | KHKVYACEVTHQGLSSPVTKSFNRGEC |
| GMCSF CDR3L | 6 | DIQMTQSPSTLSASVGDRVTITCKCQLSVGYMHWYQQKPGKA |
| VL amino acid | | PKLLIYDTSKLASGVPSRFSGSGSGTEFTLTISSLQPDDFATYYCF |
| | | 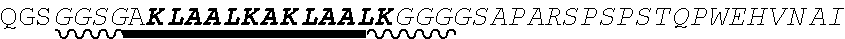 |
| | | QEARRLLNLSRDTAAEMNETVEVISEMFDLQEPTCLQTRLELYKQG |
| | | LRGSLTKLKGPLTMMASHYKQHCPPTPETSCATQIITFESFKENLK |
| | | 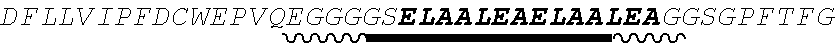 |
| | | GGTKLEIKR |
| GMCSF | 7 | TVAAPSVFIFPPSDEQLKSGTASVVCLLNNFYPREAKVQWKVD |
| CDR3L/CDR3H | | NALQSGNSQESVTEQDSKDSTYSLSSTLTLSKADYEKHKVYAC |
| CL amino acid | | EVTHQGLSSPVTKSFNRGEC |
| Linker c1 | 8 | 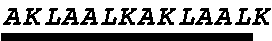 |
| Linker c2 | 9 | 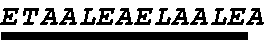 |
| Linker g1 | 10 | 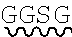 |
| Linker g2 | 11 | 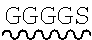 |
| Linker a | 12 | 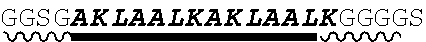 |
| Linker b | 13 | 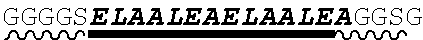 |
| GMCSF CDR3L | 14 | DIQMTQSPSTLSASVGDRVTITC |
| VL N-term aa | | |
| (FR1) | | |
| GMCSF CDR3L | 15 | FGGGTKLEIK |
| VL C-term aa | | |
| (FR4) | | |
| Human | 16 | APARSPSPSTQPWEHVNAIQEARRLLNLSRDTAAEMNETVEVISEMF |
| GMCSF | | DLQEPTCLQTRLELYKQGLRGSLTKLKGPLTMMASHYKQHCPPTP |
| | | ETSCATQIITFESFKENLKDFLLVIPFDCWEPVQE |
| Unmodified VL | 17 | DIQMTQSPSTLSASVGDRVTITCKCQLSVGYMHWYQQKPGKA |
| | | PKLLIYDTSKLASGVPSRFSGSGSGTEFTLTISSLQPDDFATYYCF |
| | | QGSGYPFTFGGGTKLEIKR |
| GMCSF CDR3L | 18 | 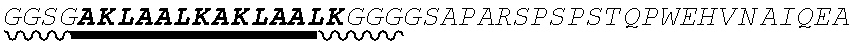 |
| insert amino | | RRLLNLSRDTAAEMNETVEVISEMFDLQEPTCLQTRLELYKQGLRG |
| acid | | SLTKLKGPLTMMASHYKQHCPPTPETSCATQIITFESFKENLKDFLL |
| | | 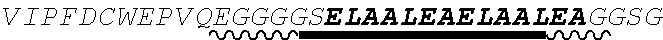 |
| GMCSF CDR3L | 19 | GFSLSTSGMSVG |
| HC CDR1 | | |
| GMCSF CDR3L | 20 | DIWWDDKKDYNPSLKS |
| HC CDR2 | | |
| GMCSF CDR3L | 21 | 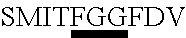 |
| HC CDR3 | | |
| GMCSF CDR3L | 22 | KCQLSVGYMH |
| LC CDR1 | | |
| GMCSF CDR3L | 23 | DTSKLAS |
| LC CDR2 | | |
| GMCSF CDR3L | 24 | 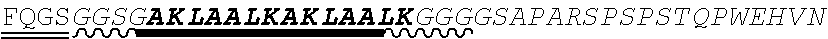 |
| LC CDR3 | | AIQEARRLLNLSRDTAAEMNETVEVISEMFDLQEPTCLQTRLELYKQ |
| | | GLRGSLTKLKGPLTMMASHYKQHCPPTPETSCATQIITFESFKENL |
| | | 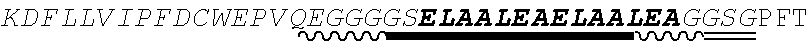 |
| Human IgGl | 25 | ASTKGPSVFPLAPSSKSTSGGTAALGCLVKDYFPEPVTVSWNSG |
| constant regions | | ALTSGVHTFPAVLQSSGLYSLSSVVTVPSSSLGTQTYICNVNHK |
| CH1-CH3 | | PSNTKVDKKVEPKSCDKTHTCPPCPAPELLGGPSVFLFPPKPKD |
| | | TLMISRTPEVTCVVVDVSHEDPEVKFNWYVDGVEVHNAKTKP |
| | | REEQYNSTYRVVSVLTVLHQDWLNGKEYKCKVSNKALPAPIE |
| | | KTISKAKGQPREPQVYTLPPSRDELTKNQVSLTCLVKGFYPSDI |
| | | AVEWESNGQPENNYKTTPPVLDSDGSFFLYSKLTVDKSRWQQ |
| | | GNVFSCSVMHEALHNHYTQKSLSLSPGK |
| CDR3L fusion 1 | 26 | DIQMTQSPSTLSASVGDRVTITCKCQLSVGYMHWYQQKPGKA |
| | | PKLLIYDTSKLASGVPSRFSGSGSGTEFTLTISSLQPDDFATYYCF |
| | | 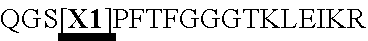 |
| CDR3L fusion 2 | 27 | DIQMTQSPSTLSASVGDRVTITCKCQLSVGYMHWYQQKPGKA |
| | | PKLLIYDTSKLASGVPSRFSGSGSGTEFTLTISSLQPDDFATYYCF |
| | | 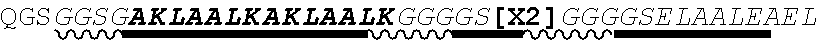 |
| | | 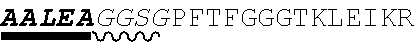 |
| GMCSF | 28 | EVQLVESGGGLVQPGGSLRLSCAASGFNIKDTYIHWVRQAPGK |
| CDR3H HC | | GLEWVARIYPTNGYTRYADSVKGRFTISADTSKNTAYLQMNSL |
| amino acid | | 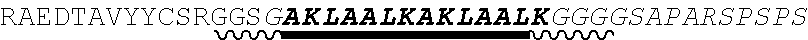 |
| | | TQPWEHVNAIQEARRLLNLSRDTAAEMNETVEVISEMFDLQEPTCL |
| | | QTRLELYKQGLRGSLTKLKGPLIMMASHYKQHCPPTPETSCATQII |
| | | 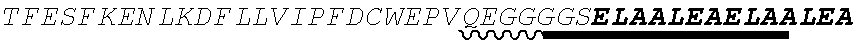 |
| | | 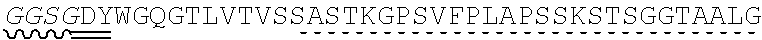 |
| | | 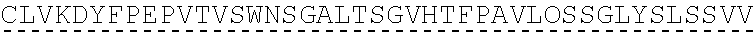 |
| | | 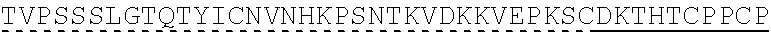 |
| | | APPVAGPSVFLFPPKPKDTLMISRTPEVTCVVVDVSHEDPEVKF |
| | | NWYVDGVEVHNAKTKPREEQYNSTYRVVSVLTVLHQDWLNG |
| | | KEYKCKVSNKGLPSSIEKTISKAKGQPREPQVYTLPPSRDELTK |
| | | NQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| | | K |
| GMCSF | 29 | EVQLVESGGGLVQPGGSLRLSCAASGFNIKDTYIHWVRQAPGK |
| CDR3H VH | | GLEWVARIYPTNGYTRYADSVKGRFTISADTSKNTAYLQMNSL |
| amino acid | | 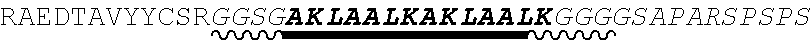 |
| | | TQPWEHVNAIQEARRLLNLSRDTAAEMNETVEVISEMFDLQEPTCL |
| | | QTRLELYKQGLRGSLTKLKGPLTMMASHYKQHCPPTPETSCATQII |
| | | 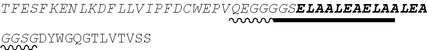 |
| GMCSF | 30 | DIQMTQSPSSLSASVGDRVTITCRASQDVNTAVAWYQQKPGKA |
| CDR3H LC | | PKLLIYSASFLYSGVPSRFSGSRSGTDFTLTISSLQPEDFATYYCQ |
| amino acid | | QHYTTPPTFGQGTKVEIKRTVAAPSVFTFPPSDEQLKSGTASVVC |
| | | LLNNFYPREAKVQWKVDNALQSGNSQESVTEQDSKDSTYSLSS |
| | | TLTLSKADYEKHKVYACEVTHQGLSSPVTKSFNRGEC |
| GMCSF | 31 | DIQMTQSPSSLSASVGDRVTITCRASQDVNTAVAWYQQKPGKA |
| CDR3H VL | | PKLLIYSASFLYSGVPSRFSGSRSGTDFTLTISSLQPEDFATYYCQ |
| amino acid | | QHYTTPPTFGQGTKVEIKR |
| GMCSF | 32 | EVQLVESGGGLVQPGGSLRLSCAAS |
| CDR3H VH N- | | |
| term aa (FR1) | | |
| GMCSF | 33 | WGQGTLVTVSS |
| CDR3H VH C- | | |
| term aa (FR4) | | |
| GMCSF | 34 | GFNIKDTYIH |
| CDR3H HC | | |
| CDR1 | | |
| GMCSF | 35 | RIYPTNGYTRYADSVKG |
| CDR3H HC | | |
| CDR2 | | |
| GMCSF | 36 | 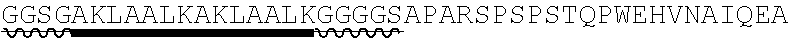 |
| CDR3H HC | | RRLLNLSRDTAAEMNETVEVISEMFDLQEPTCLQTRLELYKQGLRG |
| CDR3 | | SLTKLKGPLTMMASHYKQHCPPTPETSCATQIITFESFKENLKDFLL |
| | | 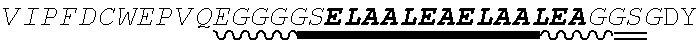 |
| GMCSF | 37 | RASQDVNTAVA |
| CDR3H LC | | |
| CDR1 | | |
| GMCSF | 38 | SASFLYS |
| CDR3H LC | | |
| CDR2 | | |
| GMCSF | 39 | QQHYTTPPT |
| CDR3H LC | | |
| CDR3 | | |
| CDR3H fusion 1 | 40 | EVQLVESGGGLVQPGGSLRLSCAASGFNIKDTYIHWVRQAPGK |
| | | GLEWVARIYPTNGYTRYADSVKGRFTISADTSKNTAYLQMNSL |
| | | RAEDTAVYYCSR[[X3]]DYWGQGTLVTVSS |
| CDR3H fusion 2 | 41 | EVQLVESGGGLVQPGGSLRLSCAASGFNIKDTYIHWVRQAPGK |
| | | GLEWVARIYPTNGYTRYADSVKGRFTISADTSKNTAYLQMNSL |
| | | 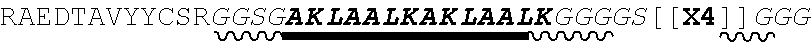 |
| | | 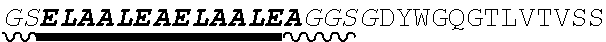 |
| CDR3H HC | 42 | EVQLVESGGGLVQPGGSLRLSCAASGFNIKDTYIHWVRQAPGK |
| scaffold amino | | GLEWVARIYPTNGYTRYADSVKGRFTISADTSKNTAYLQMNSL |
| acid | | RAEDTAVYYCSR[[X5]]WGQGTLVTVSS |
| CDR3H HC | 43 | EVQLVESGGGLVQPGGSLRLSCAASGFNIKDTYIHWVRQAPGK |
| scaffold amino | | GLEWVARIYPTNGYTRYADSVKGRFTISADTSKNTAYLQMNSL |
| acid | | 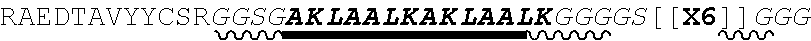 |
| | | 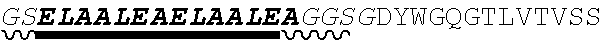 |
| Unmodified VH | 44 | EVQLVESGGGLVQPGGSLRLSCAASGFNIKDTYIHWVRQAPGK |
| | | GLEWVARIYPTNGYTRYADSVKGRFTISADTSKNTAYLQMNSL |
| | | RAEDTAVYYCSRWGGDGFYAMDYWGQGTLVTVSS |
| Syn-hGMCSF | 45 | 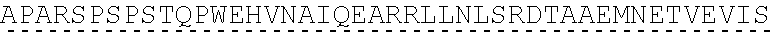 |
| NT VH(HC | | 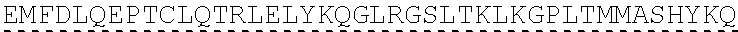 |
| fusion) | | 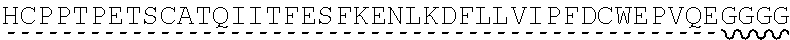 |
| | | 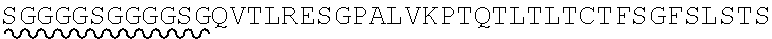 |
| | | GMSVGWIRQPPGKALEWLADIWWDDKKDYNPSLKSRLTISKD |
| | | TSKNQVVLKVTNMDPADTATYYCARSMITFGGFDVWGAGTT |
| | | VTVSS |
| Syn-hGMCSF | 46 | DIQMTQSPSTLSASVGDRVTITCKCQLSVGYMHWYQQKPGKA |
| NT VL(HC | | PKLLIYDTSKLASGVPSRFSGSGSGTAFTLTISSLQPDDFATYYC |
| fusion) | | FQGNGYPFTFGGGTKLEIKR |
| Syn-hGMCSF | 47 | 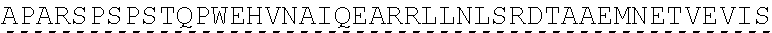 |
| NT heavy chain | | 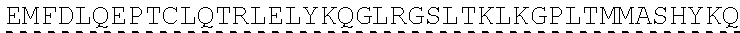 |
| (HC fusion) | | 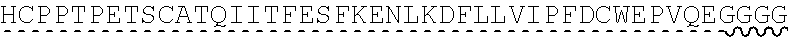 |
| | | 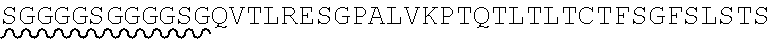 |
| | | GMSVGWIRQPPGKALEWLADIWWDDKKDYNPSLKSRLTISKD |
| | | TSKNQVVLKVTNMDPADTATYYCARSMITFGGFDVWGAGTT |
| | | VTVSSASTKGPSVFPLAPSSKSTSGGTAALGCLVKDYFPEPVTV |
| | | SWNSGALTSGVHTFPAVLQSSGLYSLSSVVTVPSSSLGTQTYIC |
| | | NVNHKPSNTKVDKKVEPKSCDKTHTCPPCPAPPVAGPSVFLFPP |
| | | KPKDTLMISRTPEVTCVVVDVSHEDPEVKFNWYVDGVEVHNA |
| | | KTKPREEQYNSTYRVVSVLTVLHQDWLNGKEYKCKVSNKGLP |
| | | SSIEKTISKAKGQPREPQVYTLPPSRDELTKNQVSLTCLVKGFYP |
| | | SDIAVEWESNGQPENNYKTTPPVLDSDGSFFLYSKLTVDKSRW |
| | | QQGNVFSCSVMHEALHNHYTQKSLSLSPGK |
| Syn-hGMCSF | 48 | DIQMTQSPSTLSASVGDRVTITCKCQLSVGYMHWYQQKPGKA |
| NT light chain | | PKLLIYDTSKLASGVPSRFSGSGSGTAFTLTISSLQPDDFATYYC |
| (HC fusion) | | FQGNGYPFTFGGGTKLEIKRTVAAPSVFIFPPSDEQLKSGTASVV |
| | | CLLNNFYPREAKVQWKVDNALQSGNSQESVTEQDSKDSTYSL |
| | | SSTLTLSKADYEKHKVYACEVTHQGLSSPVTKSFNRGEC |
| Syn-mGMCSF | 49 | 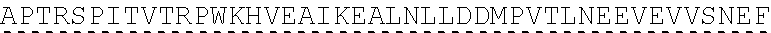 |
| NT VH(HC | | 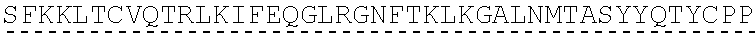 |
| fusion) | | 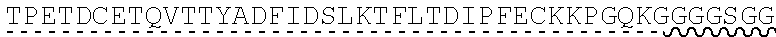 |
| | | 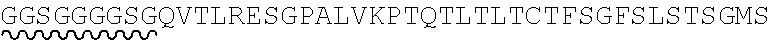 |
| | | VGWIRQPPGKALEWLADIWWDDKKDYNPSLKSRLTISKDTSK |
| | | NQVVLKVTNMDPADTATYYCARSMITFGGFDVWGAGTTVTV |
| | | SS |
| Syn-mGMCSF | 50 | DIQMTQSPSTLSASVGDRVTITCKCQLSVGYMHWYQQKPGKA |
| NT VL(HC | | PKLLIYDTSKLASGVPSRFSGSGSGTAFTLTISSLQPDDFATYYC |
| fusion) | | FQGNGYPFTFGGGTKLEIKR |
| Syn-mGMCSF | 51 | 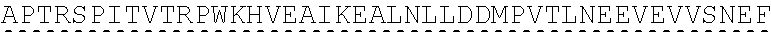 |
| NT heavy chain | | 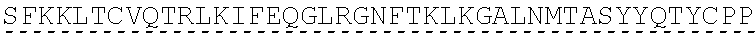 |
| (HC fusion) | | 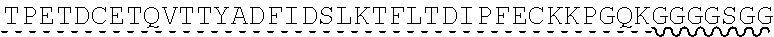 |
| | | 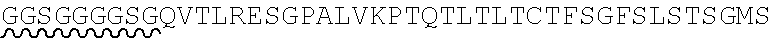 |
| | | VGWIRQPPGKALEWLADIWWDDKKDYNPSLKSRLTISKDTSK |
| | | NQVVLKVTNMDPADTATYYCARSMITFGGFDVWGAGTTVTV |
| | | SSASTKGPSVFPLAPSSKSTSGGTAALGCLVKDYFPEPVTVSWN |
| | | SGALTSGVHTFPAVLQSSGLYSLSSWTVPSSSLGTQTYICNVN |
| | | HKPSNTKVDKKVEPKSCDKTHTCPPCPAPPVAGPSVFLFPPKPK |
| | | DTLMISRTPEVTCVVVDVSHEDPEVKFNWYVDGVEVHNAKTK |
| | | PREEQYNSTYRVVSVLTVLHQDWLNGKEYKCKVSNKGLPSSIE |
| | | KTISKAKGQPREPQVYTLPPSRDELTKNQVSLTCLVKGFYPSDI |
| | | AVEWESNGQPENNYKTTPPVLDSDGSFFLYSKLTVDKSRWQQ |
| | | GNVFSCSVMHEALHNHYTQKSLSLSPGK |
| Syn-mGMCSF | 52 | DIQMTQSPSTLSASVGDRVTITCKCQLSVGYMHWYQQKPGKA |
| NT light chain | | PKLLIYDTSKLASGVPSRFSGSGSGTAFTLTISSLQPDDFATYYC |
| (HC fusion) | | FQGNGYPFTFGGGTKLEIKRTVAAPSVFIFPPSDEQLKSGTASVV |
| | | CLLNNFYPREAKVQWKVDNALQSGNSQESVTEQDSKDSTYSL |
| | | SSTLTLSKADYEKHKVYACEVTHQGLSSPVTKSFNRGEC |
| Her-mGMCSF | 53 | EVQLVESGGGLVQPGGSLRLSCAASGFNIKDTYIHWVRQAPGK |
| CDRVH | | GLEWVARIYPTNGYTRYADSVKGRFTISADTSKNTAYLQMNSL |
| | | 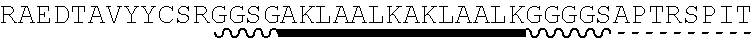 |
| | | 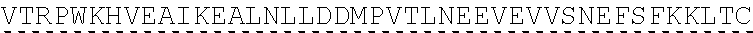 |
| | | 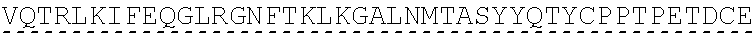 |
| | | 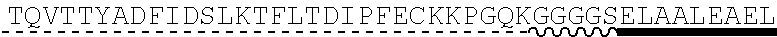 |
| | | 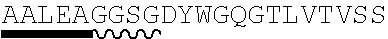 |
| Her-mGMCSF | 54 | DIQMTQSPSSLSASVGDRVTITCRASQDVNTAVAWYQQKPGKA |
| CDRVL | | PKLLIYSASFLYSGVPSRFSGSRSGTDFTLTISSLQPEDFATYYCQ |
| | | QHYTTPPTFGQGTKVEIKR |
| Her-mGMCSF | 55 | EVQLVESGGGLVQPGGSLRLSCAASGFNIKDTYIHWVRQAPGK |
| CDR heavy | | GLEWVARIYPTNGYTRYADSVKGRFTISADTSKNTAYLQMNSL |
| chain | | 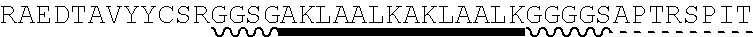 |
| | | 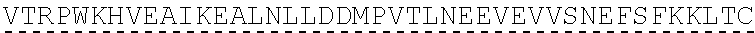 |
| | | 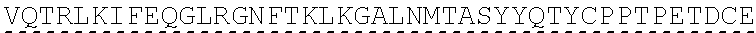 |
| | | 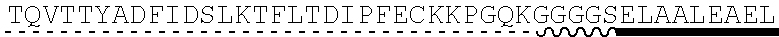 |
| | | 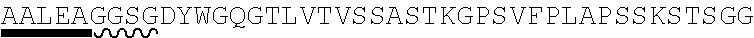 |
| | | TAALGCLVKDYFPEPVTVSWNSGALTSGVHTFPAVLQSSGLYS |
| | | LSSVVTVPSSSLGTQTYICNVNHKPSNTKVDKKVEPKSCDKTHT |
| | | CPPCPAPPVAGPSVFLFPPKPKDTLMISRTPEVTCVVVDVSHEDP |
| | | EVKFNWYVDGVEVHNAKTKPREEQYNSTYRVVSVLTVLHQD |
| | | WLNGKEYKCKVSNKGLPSSIEKTISKAKGQPREPQVYTLPPSRD |
| | | ELTKNQVSLTCLVKGFYPSDIAVEWESNGQPENNYKTTPPVLD |
| | | SDGSFFLYSKLTVDKSRWQQGNVFSCSVMHEALHNHYTQKSL |
| | | SLSPGK |
| Her-mGMCSF | 56 | DIQMTQSPSSLSASVGDRVTITCRASQDVNTAVAWYQQKPGKA |
| CDR light chain | | PKTIIYSASFLYSGVPSRFSGSRSGTDFTTTISSLQPEDFATYYCQ |
| | | QHYTTPPTFGQGTKVEIKRTVAAPSVFIFPPSDEQLKSGTASVVC |
| | | LLNNFYPREAKVQWKVDNALQSGNSQESVTEQDSKDSTYSLSS |
| | | TLTLSKADYEKHKVYACEVTHQGLSSPVTKSFNRGEC |
| Syn-mGMCSF | 57 | QVTLRESGPALVKPTQTLTLTCTFSGFSLSTSGMSVGWIRQPPG |
| CDR VH | | KALEWLADIWWDDKKDYNPSLKSRLTISKDTSKNQVVLKVTN |
| | | 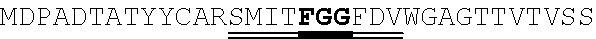 |
| Syn-mGMCSF | 58 | DIQMTQSPSTLSASVGDRVTITCKCQLSVGYMHWYQQKPGKA |
| CDR VL | | PKLLIYDTSKLASGVPSRFSGSGSGTAFTLTISSLQPDDFATYYC |
| | | 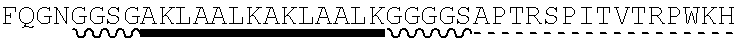 |
| | | 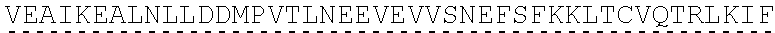 |
| | | 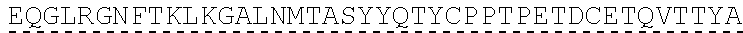 |
| | | 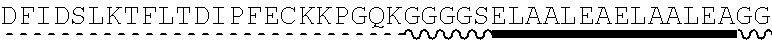 |
| | | 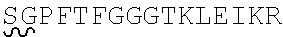 |
| Syn-mGMCSF | 59 | QVTLRESGPALVKPTQTLTLTCTFSGFSLSTSGMSVGWIRQPPG |
| CDR heavy | | KALEWLADIWWDDKKDYNPSLKSRLTISKDTSKNQVVLKVTN |
| chain | | 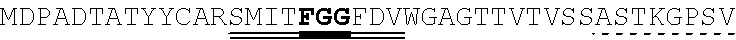 |
| | | 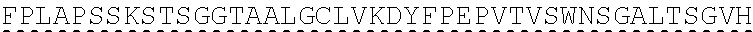 |
| | | 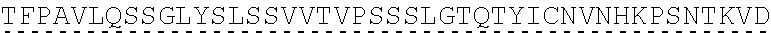 |
| | | 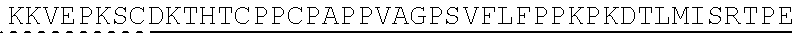 |
| | | VTCVVVDVSHEDPEVKFNWYVDGVEVHNAKTKPREEOYNST |
| | | YRVVSVLTVLHQDWLNGKEYKCKVSNKGLPSSIEKTISKAKGQ |
| | | PREPQVYTLPPSRDELTKNQVSLTCLVKGFYPSDIAVEWESNGQ |
| | | PENNYKTTPPVLDSDGSFFLYSKLTVDKSRWQQGNVFSCSVMH |
| | | EALHNHYTQKSLSLSPGK |
| Syn-mGMCSF | 60 | DIQMTQSPSTLSASVGDRVTITCKCQLSVGYMHWYQQKPGKA |
| CDR light chain | | PKLLIYDTSKLASGVPSRFSGSGSGTAFTLTISSLQPDDFATYYC |
| | | 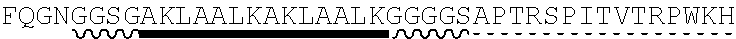 |
| | | 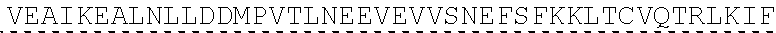 |
| | | 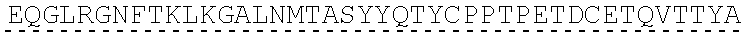 |
| | | 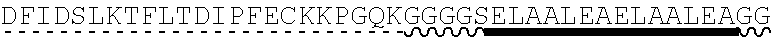 |
| | | 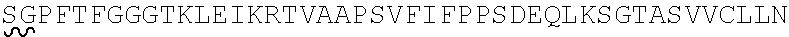 |
| | | NFYPREAKVQWKVDNALQSGNSQESVTEQDSKDSTYSLSSTLT |
| | | LSKADYEKHKVYACEVTHQGLSSPVTKSFNRGEC |
| Syn-hGMCSF | 61 | QVTLRESGPALVKPTQTLTLTCTFSGFSLSTSGMSVGWIRQPPG |
| NT VH(LC | | KALEWLADIWWDDKKDYNPSLKSRLTISKDTSKNQVVLKVTN |
| fusion) | | 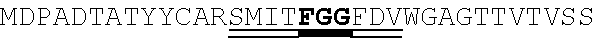 |
| Syn-hGMCSF | 62 | 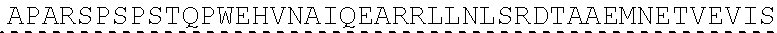 |
| NT VL(LC | | 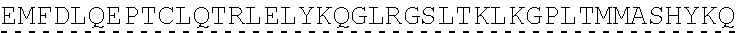 |
| fusion) | | 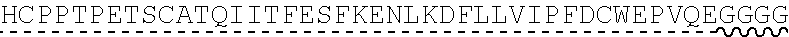 |
| | | 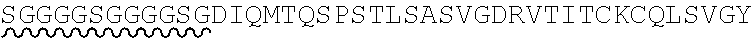 |
| | | MHWYQQKPGKAPKLLIYDTSKLASGVPSRFSGSGSGTAFTLTIS |
| | | SLQPDDFATYYCFQGNGYPFTFGGGTKLEIKR |
| Syn-hGMCSF | 63 | QVTLRESGPALVKPTQTLTLTCTFSGFSLSTSGMSVGWIRQPPG |
| NT heavy chain | | KALEWLADIWWDDKKDYNPSLKSRLTISKDTSKNQVVLKVTN |
| (LC fusion) | | 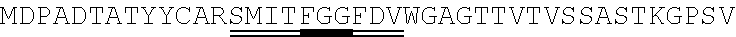 |
| | | 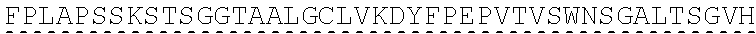 |
| | | 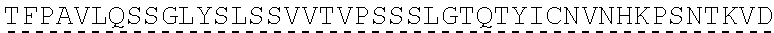 |
| | | 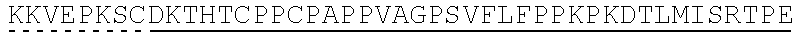 |
| | | VTCVVVDVSHEDPEVKFNWYVDGVEVHNAKTKPREEQYNST |
| | | YRVVSVLTVLHQDWLNGKEYKCKVSNKGLPSSIEKTISKAKGQ |
| | | PREPQVYTLPPSRDELTKNOVSLTCLVKGFYPSDIAVEWESNGQ |
| | | PENNYKTTPPVLDSDGSFFLYSKLTVDKSRWQQGNVFSCSVMH |
| | | EALHNHYTQKSLSLSPGK |
| Syn-hGMCSF | 64 | 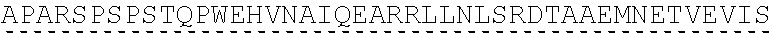 |
| NT light chain | | 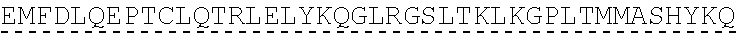 |
| (LC fusion) | | 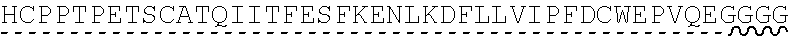 |
| | | 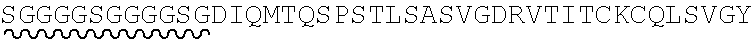 |
| | | MHWYQQKPGKAPKLLIYDTSKLASGVPSRFSGSGSGTAFTLTIS |
| | | SLQPDDFATYYCFQGNGYPFTFGGGTKLEIKRTVAAPSVFIFPPS |
| | | DEQLKSGTASVVCLLNNFYPREAKVQWKVDNALQSGNSQESV |
| | | TEQDSKDSTYSLSSTLTLSKADYEKHKVYACEVTHQGLSSPVT |
| | | KSFNRGEC |
| Unmodified VH | 65 | QVTLRESGPALVKPTQTLTLTCTFSGFSLSTSGMSVGWIRQPPG |
| | | KALEWLADIWWDDKKDYNPSLKSRLTISKDTSKNQVVLKVTN |
| | | MDPADTATYYCARSMITNWYFDVWGAGTTVTVSS |
| Palivizumab HC | 66 | SMITX(i)X(ii)X(iii)FDV |
| CDR3 | | wherein X(i) is selected from F, A, G, and P; |
| | | X(ii) is selected from G, A, S, T, and P; |
| | | and X(iii) is selected from G, A, V, L, and P |
| hGMCSF-hlgGl | 67 | 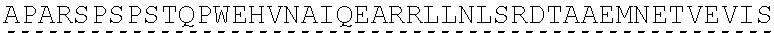 |
| Fc | | 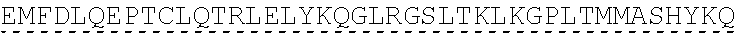 |
| | | 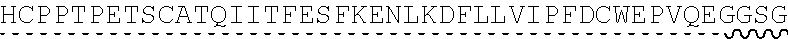 |
| | | 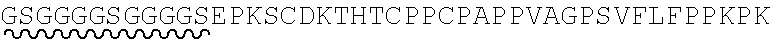 |
| | | DTLMISRTPEVTCVVVDVSHEDPEVKFNWYVDGVEVHNAKTK |
| | | PREEQYNSTYRVVSVLTVLHQDWLNGKEYKCKVSNKGLPSSIE |
| | | KTISKAKGQPREPQVYTLPPSRDELTKNQVSLTCLVKGFYPSDI |
| | | AVEWESNGQPENNYKTTPPVLDSDGSFFLYSKLTVDKSRWQQ |
| | | GNVFSCSVMHEALHNHYTQKSLSLSPGK |
| mGMCSF- | 68 | 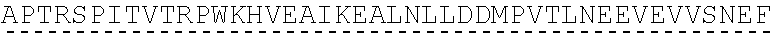 |
| hIgG1 Fc | | 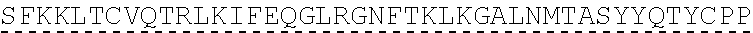 |
| | | 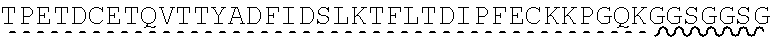 |
| | | 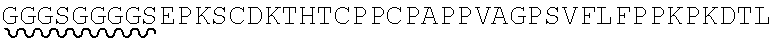 |
| | | MISRTPEVTCVVVDVSHEDPEVKFNWYVDGVEVHNAKTKPRE |
| | | EQYNSTYRVVSVLTVLHQDWLNGKEYKCKVSNKGLPSSIEKTI |
| | | SKAKGQPREPQVYTLPPSRDELTKNQVSLTCLVKGFYPSDIAVE |
| | | WESNGQPENNYKTTPPVLDSDGSFFLYSKLTVDKSRWQQGNV |
| | | FSCSVMHEALHNHYTQKSLSLSPGK |
| nucleotide | 69 | GAGGTGCAGCTGGTGGAGTCTGGAGGAGGCTTGGTCCAGCC |
| sequences of | | TGGGGGGTCCCTGAGACTCTCCTGTGCAGCCTCTGGGTTCAA |
| Herception | | TATTAAGGACACTTACATCCACTGGGTCCGCCAGGCTCCAGG |
| hGMCSF CDR | | GAAGGGGCTGGAGTGGGTCGCACGTATTTATCCTACCAATG |
| heavy chain | | GTTACACACGCTACGCAGACTCCGTGAAGGGCCGATTCACC |
| (HC fusion) | | ATCTCCGCAGACACTTCCAAGAACACGGCGTATCTTCAAATG |
| | | AACAGCCTGAGAGCCGAGGACACGGCCGTGTATTACTGTTC |
| | | GAGAGGCGGAAGCGGAGCAAAGCTCGCCGCACTGAAAGCC |
| | | AAGCTGGCCGCTCTGAAGGGAGGTGGCGGGAGCGCACCCGC |
| | | CCGCTCGCCCAGCCCCAGCACGCAGCCCTGGGAGCATGTGA |
| | | ATGCCATCCAGGAGGCCCGGCGTCTCCTGAACCTGAGTAGA |
| | | GACACTGCTGCTGAGATGAATGAAACAGTAGAAGTCATCTC |
| | | AGAAATGTTTGACCTCCAGGAGCCGACCTGCCTACAGACCC |
| | | GCCTGGAGCTGTACAAGCAGGGCCTGCGGGGCAGCCTCACC |
| | | AAGCTCAAGGGCCCCTTGACCATGATGGCCAGCCACTACAA |
| | | GCAGCACTGCCCTCCAACCCCGGAAACTTCCTGTGCAACCCA |
| | | GATTATCACCTTTGAAAGTTTCAAAGAGAACCTGAAGGACTT |
| | | TCTGCTTGTCATCCCCTTTGACTGCTGGGAGCCAGTCCAGGA |
| | | GGGCGGAGGTGGGAGTGAACTGGCCGCACTGGAAGCTGAGC |
| | | TGGCTGCCCTCGAAGCTGGAGGCTCTGGAGACTACTGGGGC |
| | | CAAGGAACCCTGGTCACCGTCTCCTCAGCCTCCACCAAGGG |
| | | CCCATCGGTCTTCCCCCTGGCACCCTCCTCCAAGAGCACCTC |
| | | TGGGGGCACAGCGGCCCTGGGCTGCCTGGTCAAGGACTACT |
| | | TCCCCGAACCGGTGACGGTGTCGTGGAACTCAGGCGCCCTG |
| | | ACCAGCGGCGTGCACACCTTCCCGGCTGTCCTACAGTCCTCA |
| | | GGACTCTACTCCCTCAGCAGCGTGGTGACTGTGCCCTCTAGC |
| | | AGCTTGGGCACCCAGACCTACATCTGCAACGTGAATCACAA |
| | | GCCCAGCAACACCAAGGTGGACAAGAAAGTTGAACCCAAAT |
| | | CTTGCGACAAAACTCACACATGCCCACCGTGCCCAGCACCTC |
| | | CAGTCGCCGGACCGTCAGTCTTCCTCTTCCCTCCAAAACCCA |
| | | AGGACACCCTCATGATCTCCCGGACCCCTGAGGTCACATGC |
| | | GTGGTGGTGGACGTGAGCCACGAAGACCCTGAGGTCAAGTT |
| | | CAACTGGTACGTGGACGGCGTGGAGGTGCATAATGCCAAGA |
| | | CAAAGCCGCGGGAGGAGCAGTACAACAGCACGTACCGTGTG |
| | | GTCAGCGTCCTCACCGTCCTGCACCAGGACTGGCTGAATGGC |
| | | AAGGAGTACAAGTGCAAGGTCTCCAACAAAGGCCTCCCAAG |
| | | CTCCATCGAGAAAACCATCTCCAAAGCCAAAGGGCAGCCCC |
| | | GAGAACCACAGGTGTACACCCTGCCTCCATCCCGGGATGAG |
| | | CTGACCAAGAACCAGGTCAGCCTGACCTGCCTGGTCAAAGG |
| | | CTTCTATCCCAGCGACATCGCCGTGGAGTGGGAGAGCAATG |
| | | GGCAGCCGGAGAACAACTACAAGACCACGCCTCCCGTGCTG |
| | | GACTCCGACGGCTCCTTCTTCCTCTACAGCAAGCTCACCGTG |
| | | GACAAGAGCAGGTGGCAGCAGGGGAACGTCTTCTCATGCTC |
| | | CGTGATGCATGAGGCTCTGCACAACCACTACACGCAGAAGA |
| | | GCCTCTCCCTGTCTCCGGGTAAA |
| nucleotide | 70 | GACATCCAGATGACCCAGTCTCCATCCTCCCTGTCTGCATCT |
| sequences of | | GTAGGAGACAGAGTCACCATCACTTGCCGGGCAAGTCAGGA |
| Herception | | TGTGAATACCGCGGTCGCATGGTATCAGCAGAAACCAGGGA |
| hGMCSF CDR | | AAGCCCCTAAGCTCCTGATCTATTCTGCATCCTTCTTGTATA |
| light chain(HC | | GTGGGGTCCCATCAAGGTTCAGTGGCAGTAGATCTGGGACA |
| fusion) | | GATTTCACTCTCACCATCAGCAGTCTGCAACCTGAAGATTTT |
| | | GCAACTTACTACTGTCAACAGCATTACACTACCCCTCCGACG |
| | | TTCGGCCAAGGTACCAAGGTGGAGATCAAACGAACTGTGGC |
| | | TGCACCATCTGTCTTCATCTTCCCGCCATCTGATGAGCAGTT |
| | | GAAATCTGGAACTGCCTCTGTCGTGTGCCTGCTGAATAACTT |
| | | CTATCCCAGAGAGGCCAAAGTACAGTGGAAGGTGGATAACG |
| | | CCCTCCAATCGGGTAACTCCCAGGAGAGTGTCACAGAGCAG |
| | | GACAGCAAGGACAGCACCTACAGCCTCAGCAGCACCCTGAC |
| | | GCTGAGCAAAGCAGACTACGAGAAACACAAAGTCTACGCCT |
| | | GCGAAGTCACCCATCAGGGCCTGTCCTCGCCCGTCACAAAG |
| | | AGCTTCAACAGGGGAGAGTGT |
| nucleotide | 71 | CAGGTGACCCTGCGCGAGTCCGGCCCTGCACTGGTGAAGCC |
| sequences of | | CACCCAGACCCTGACCCTGACCTGCACCTTCTCCGGCTTCTC |
| Synagis | | CCTGTCCACCTCCGGCATGTCCGTGGGCTGGATCCGGCAGCC |
| hGMCSF CDR | | TCCCGGCAAGGCCCTGGAGTGGCTGGCTGACATCTGGTGGG |
| heavy chain | | ACGACAAGAAGGACTACAACCCCTCCCTGAAGTCCCGCCTG |
| (LC fusion) | | ACCATCTCCAAGGACACCTCCAAGAACCAGGTGGTGCTGAA |
| | | GGTGACCAACATGGACCCCGCCGACACCGCCACCTACTACT |
| | | GCGCCCGCTCAATGATTACCTTCGGGGGCTTCGACGTGTGGG |
| | | GAGCCGGTACCACCGTGACCGTGTCTTCCGCCTCCACCAAGG |
| | | GCCCATCGGTCTTCCCCCTGGCACCCTCCTCCAAGAGCACCT |
| | | CTGGGGGCACAGCGGCCCTGGGCTGCCTGGTCAAGGACTAC |
| | | TTCCCCGAACCGGTGACGGTGTCGTGGAACTCAGGCGCCCT |
| | | GACCAGCGGCGTGCACACCTTCCCGGCTGTCCTACAGTCCTC |
| | | AGGACTCTACTCCCTCAGCAGCGTGGTGACTGTGCCCTCTAG |
| | | CAGCTTGGGCACCCAGACCTACATCTGCAACGTGAATCACA |
| | | AGCCCAGCAACACCAAGGTGGACAAGAAAGTTGAACCCAA |
| | | ATCTTGCGACAAAACTCACACATGCCCACCGTGCCCAGCAC |
| | | CTCCAGTCGCCGGACCGTCAGTCTTCCTCTTCCCTCCAAAAC |
| | | CCAAGGACACCCTCATGATCTCCCGGACCCCTGAGGTCACAT |
| | | GCGTGGTGGTGGACGTGAGCCACGAAGACCCTGAGGTCAAG |
| | | TTCAACTGGTACGTGGACGGCGTGGAGGTGCATAATGCCAA |
| | | GACAAAGCCGCGGGAGGAGCAGTACAACAGCACGTACCGT |
| | | GTGGTCAGCGTCCTCACCGTCCTGCACCAGGACTGGCTGAAT |
| | | GGCAAGGAGTACAAGTGCAAGGTCTCCAACAAAGGCCTCCC |
| | | AAGCTCCATCGAGAAAACCATCTCCAAAGCCAAAGGGCAGC |
| | | CCCGAGAACCACAGGTGTACACCCTGCCTCCATCCCGGGAT |
| | | GAGCTGACCAAGAACCAGGTCAGCCTGACCTGCCTGGTCAA |
| | | AGGCTTCTATCCCAGCGACATCGCCGTGGAGTGGGAGAGCA |
| | | ATGGGCAGCCGGAGAACAACTACAAGACCACGCCTCCCGTG |
| | | CTGGACTCCGACGGCTCCTTCTTCCTCTACAGCAAGCTCACC |
| | | GTGGACAAGAGCAGGTGGCAGCAGGGGAACGTCTTCTCATG |
| | | CTCCGTGATGCATGAGGCTCTGCACAACCACTACACGCAGA |
| | | AGAGCCTCTCCCTGTCTCCGGGTAAA |
| nucleotide | 72 | GACATCCAGATGACCCAGTCCCCCTCCACCCTGTCCGCCTCC |
| sequences of | | GTGGGCGACCGCGTGACCATCACCTGCAAGTGCCAGCTGTC |
| Synagis | | CGTGGGCTACATGCACTGGTACCAGCAGAAGCCCGGCAAGG |
| hGMCSF CDR | | CCCCCAAGCTGCTGATCTACGACACCTCCAAGCTGGCCTCCG |
| light chain(LC | | GCGTGCCCTCCCGCTTCTCCGGCTCCGGCTCCGGCACCGAGT |
| fusion) | | TCACCCTGACCATCTCCTCCCTGCAGCCCGACGACTTCGCCA |
| | | CCTACTACTGCTTCCAGGGCTCCGGCGGAAGCGGAGCAAAG |
| | | CTCGCCGCACTGAAAGCCAAGCTGGCCGCTCTGAAGGGAGG |
| | | TGGCGGGAGCGCACCCGCCCGCTCGCCCAGCCCCAGCACGC |
| | | AGCCCTGGGAGCATGTGAATGCCATCCAGGAGGCCCGGCGT |
| | | CTCCTGAACCTGAGTAGAGACACTGCTGCTGAGATGAATGA |
| | | AACAGTAGAAGTCATCTCAGAAATGTTTGACCTCCAGGAGC |
| | | CGACCTGCCTACAGACCCGCCTGGAGCTGTACAAGCAGGGC |
| | | CTGCGGGGCAGCCTCACCAAGCTCAAGGGCCCCTTGACCAT |
| | | GATGGCCAGCCACTACAAGCAGCACTGCCCTCCAACCCCGG |
| | | AAACTTCCTGTGCAACCCAGATTATCACCTTTGAAAGTTTCA |
| | | AAGAGAACCTGAAGGACTTTCTGCTTGTCATCCCCTTTGACT |
| | | GCTGGGAGCCAGTCCAGGAGGGCGGAGGTGGGAGTGAACT |
| | | GGCCGCACTGGAAGCTGAGCTGGCTGCCCTCGAAGCTGGAG |
| | | GCTCTGGACCCTTCACCTTCGGCGGCGGCACCAAGCTGGAG |
| | | ATCAAACGAACTGTGGCTGCACCATCTGTCTTCATCTTCCCG |
| | | CCATCTGATGAGCAGTTGAAATCTGGAACTGCCTCTGTCGTG |
| | | TGCCTGCTGAATAACTTCTATCCCAGAGAGGCCAAAGTACA |
| | | GTGGAAGGTGGATAACGCCCTCCAATCGGGTAACTCCCAGG |
| | | AGAGTGTCACAGAGCAGGACAGCAAGGACAGCACCTACAG |
| | | CCTCAGCAGCACCCTGACGCTGAGCAAAGCAGACTACGAGA |
| | | AACACAAAGTCTACGCCTGCGAAGTCACCCATCAGGGCCTG |
| | | TCCTCGCCCGTCACAAAGAGCTTCAACAGGGGAGAGTGT |
| nucleotide | 73 | GAGGTGCAGCTGGTGGAGTCTGGAGGAGGCTTGGTCCAGCC |
| sequences of | | TGGGGGGTCCCTGAGACTCTCCTGTGCAGCCTCTGGGTTCAA |
| Herception | | TATTAAGGACACTTACATCCACTGGGTCCGCCAGGCTCCAGG |
| hGMCSF CDR | | GAAGGGGCTGGAGTGGGTCGCACGTATTTATCCTACCAATG |
| VH(HC | | GTTACACACGCTACGCAGACTCCGTGAAGGGCCGATTCACC |
| fusion) | | ATCTCCGCAGACACTTCCAAGAACACGGCGTATCTTCAAATG |
| | | AACAGCCTGAGAGCCGAGGACACGGCCGTGTATTACTGTTC |
| | | GAGAGGCGGAAGCGGAGCAAAGCTCGCCGCACTGAAAGCC |
| | | AAGCTGGCCGCTCTGAAGGGAGGTGGCGGGAGCGCACCCGC |
| | | CCGCTCGCCCAGCCCCAGCACGCAGCCCTGGGAGCATGTGA |
| | | ATGCCATCCAGGAGGCCCGGCGTCTCCTGAACCTGAGTAGA |
| | | GACACTGCTGCTGAGATGAATGAAACAGTAGAAGTCATCTC |
| | | AGAAATGTTTGACCTCCAGGAGCCGACCTGCCTACAGACCC |
| | | GCCTGGAGCTGTACAAGCAGGGCCTGCGGGGCAGCCTCACC |
| | | AAGCTCAAGGGCCCCTTGACCATGATGGCCAGCCACTACAA |
| | | GCAGCACTGCCCTCCAACCCCGGAAACTTCCTGTGCAACCCA |
| | | GATTATCACCTTTGAAAGTTTCAAAGAGAACCTGAAGGACTT |
| | | TCTGCTTGTCATCCCCTTTGACTGCTGGGAGCCAGTCCAGGA |
| | | GGGCGGAGGTGGGAGTGAACTGGCCGCACTGGAAGCTGAGC |
| | | TGGCTGCCCTCGAAGCTGGAGGCTCTGGAGACTACTGGGGC |
| | | CAAGGAACCCTGGTCACCGTCTCCTCA |
| nucleotide | 74 | GACATCCAGATGACCCAGTCTCCATCCTCCCTGTCTGCATCT |
| sequences of | | GTAGGAGACAGAGTCACCATCACTTGCCGGGCAAGTCAGGA |
| Herception | | TGTGAATACCGCGGTCGCATGGTATCAGCAGAAACCAGGGA |
| hGMCSF CDR | | AAGCCCCTAAGCTCCTGATCTATTCTGCATCCTTCTTGTATA |
| VL chain(HC | | GTGGGGTCCCATCAAGGTTCAGTGGCAGTAGATCTGGGACA |
| fusion) | | GATTTCACTCTCACCATCAGCAGTCTGCAACCTGAAGATTTT |
| | | GCAACTTACTACTGTCAACAGCATTACACTACCCCTCCGACG |
| | | TTCGGCCAAGGTACCAAGGTGGAGATCAAACGA |
| nucleotide | 75 | CAGGTGACCCTGCGCGAGTCCGGCCCTGCACTGGTGAAGCC |
| sequences of | | CACCCAGACCCTGACCCTGACCTGCACCTTCTCCGGCTTCTC |
| Synagis | | CCTGTCCACCTCCGGCATGTCCGTGGGCTGGATCCGGCAGCC |
| hGMCSF CDR | | TCCCGGCAAGGCCCTGGAGTGGCTGGCTGACATCTGGTGGG |
| VH(LC | | ACGACAAGAAGGACTACAACCCCTCCCTGAAGTCCCGCCTG |
| fusion) | | ACCATCTCCAAGGACACCTCCAAGAACCAGGTGGTGCTGAA |
| | | GGTGACCAACATGGACCCCGCCGACACCGCCACCTACTACT |
| | | GCGCCCGCTCAATGATTACCTTCGGGGGCTTCGACGTGTGGG |
| | | GAGCCGGTACCACCGTGACCGTGTCTTCC |
| nucleotide | 76 | GACATCCAGATGACCCAGTCCCCCTCCACCCTGTCCGCCTCC |
| sequences of | | GTGGGCGACCGCGTGACCATCACCTGCAAGTGCCAGCTGTC |
| Synagis | | CGTGGGCTACATGCACTGGTACCAGCAGAAGCCCGGCAAGG |
| hGMCSF CDR | | CCCCCAAGCTGCTGATCTACGACACCTCCAAGCTGGCCTCCG |
| VL(LC fusion) | | GCGTGCCCTCCCGCTTCTCCGGCTCCGGCTCCGGCACCGAGT |
| | | TCACCCTGACCATCTCCTCCCTGCAGCCCGACGACTTCGCCA |
| | | CCTACTACTGCTTCCAGGGCTCCGGCGGAAGCGGAGCAAAG |
| | | CTCGCCGCACTGAAAGCCAAGCTGGCCGCTCTGAAGGGAGG |
| | | TGGCGGGAGCGCACCCGCCCGCTCGCCCAGCCCCAGCACGC |
| | | AGCCCTGGGAGCATGTGAATGCCATCCAGGAGGCCCGGCGT |
| | | CTCCTGAACCTGAGTAGAGACACTGCTGCTGAGATGAATGA |
| | | AACAGTAGAAGTCATCTCAGAAATGTTTGACCTCCAGGAGC |
| | | CGACCTGCCTACAGACCCGCCTGGAGCTGTACAAGCAGGGC |
| | | CTGCGGGGCAGCCTCACCAAGCTCAAGGGCCCCTTGACCAT |
| | | GATGGCCAGCCACTACAAGCAGCACTGCCCTCCAACCCCGG |
| | | AAACTTCCTGTGCAACCCAGATTATCACCTTTGAAAGTTTCA |
| | | AAGAGAACCTGAAGGACTTTCTGCTTGTCATCCCCTTTGACT |
| | | GCTGGGAGCCAGTCCAGGAGGGCGGAGGTGGGAGTGAACT |
| | | GGCCGCACTGGAAGCTGAGCTGGCTGCCCTCGAAGCTGGAG |
| | | GCTCTGGACCCTTCACCTTCGGCGGCGGCACCAAGCTGGAG |
| | | ATCAAACGA |

What is claimed is:

1. A composition comprising a first polypeptide comprising a granulocyte macrophage colony stimulating factor (GM-CSF) and a second polypeptide comprising a sequence at least 98% identical to SEQ ID NO: 2.

2. The composition of claim 1, wherein GM-CSF comprises a sequence at least about 85%, 86%, 87%, 88%, 89%, 90%, 91%, 92%, 93%, 94%, 95%, 96%, 97%, 98%, or 99% identical to SEQ ID NO: 16 or 77.

3. The composition of claim 1 or claim 2, wherein the GM-CSF comprises human GM-CSF or murine GM-CSF.

4. The composition of any one of claims 1-3, wherein the first polypeptide comprises a modified light chain of an antibody variable region.

5. The composition of claim 4, wherein the modified light chain of the antibody variable domain comprises the GM-CSF positioned between a first amino acid sequence of the antibody variable region and a second amino acid sequence of the antibody variable region.

6. The composition of claim 5, wherein the first amino acid sequence comprises a sequence at least about 90%, 91%, 92%, 93%, 94%, 95%, 96%, 97%, 98%, or 99% identical to SEQ ID NO: 14.

7. The composition of claim 6, wherein the first amino acid sequence comprises SEQ ID NO: 14.

8. The composition of any one of claims 5-7, wherein the second amino acid sequence comprises a sequence at least about 90%, 91%, 92%, 93%, 94%, 95%, 96%, 97%, 98%, or 99% identical to SEQ ID NO: 15.

9. The composition of claim 8, wherein the second amino acid sequence comprises SEQ ID NO: 15.

10. The composition of any one of claims 4-9, wherein the GM-CSF is positioned within a complementarity determining region (CDR) of the modified light chain.

11. The composition of claim 10, wherein the GM-CSF is position within light chain CDR1, CDR2, or CDR3.

12. The composition of claim 11, wherein the GM-CSF is positioned within light chain CDR3.

13. The composition of any one of claims 4-12, wherein the modified light chain is modified from a variable light chain comprising SEQ ID NO: 17.

14. The composition of any one of claims 1-13, wherein the first polypeptide further comprises a first linker peptide.

15. The composition of claim 14, wherein the first linker peptide comprises SEQ ID NO: 10.

16. The composition of claim 14 or claim 15, wherein the first linker peptide comprises SEQ ID NO: 8.

17. The composition of any one of claims 14-16, wherein the first linker peptide comprises SEQ ID NO: 11.

18. The composition of any one of claims 14-17, wherein the first linker peptide comprises a sequence at least about 90%, 91%, 92%, 93%, 94%, 95%, 96%, 97%, 98%, 99% or 100% identical to SEQ ID NO: 12.

19. The composition of any one of claims 1-18, wherein the first polypeptide further comprises a second linker peptide.

20. The composition of claim 19, wherein the second linker peptide comprises SEQ ID NO: 10.

21. The composition of claim 19 or claim 20, wherein the second linker peptide comprises SEQ ID NO: 9.

22. The composition of any one of claims 19-21, wherein the second linker peptide comprises SEQ ID NO: 11.

23. The composition of any one of claims 19-22, wherein the second linker peptide comprises a sequence at least about 90%, 91%, 92%, 93%, 94%, 95%, 96%, 97%, 98%, 99%, or 100% identical to SEQ ID NO: 13.

24. The composition of any one of claims 1-23, wherein the first polypeptide comprises a sequence at least about 90%, 91%, 92%, 93%, 94%, 95%, 96%, 97%, 98%, 99%, or 100% identical to SEQ ID NO: 18.

25. The composition of any one of claims 1-24, wherein the first polypeptide comprises a sequence at least about 90%, 91%, 92%, 93%, 94%, 95%, 96%, 97%, 98%, 99%, or 100% identical to SEQ ID NO: 6.

26. The composition of any one of claims 1-25, wherein the first polypeptide comprises a sequence at least about 90%, 91%, 92%, 93%, 94%, 95%, 96%, 97%, 98%, 99%, or 100% identical to SEQ ID NO: 7.

27. The composition of any one of claims 1-26, wherein the first polypeptide comprises a sequence at least about 90%, 91%, 92%, 93%, 94%, 95%, 96%, 97%, 98%, 99%, or 100% identical to SEQ ID NO: 5.

28. The composition of any one of claims 1-27, wherein the second polypeptide comprises a heavy chain of an antibody variable region.

29. The composition of any one of claims 1-28, wherein the second polypeptide comprises SEQ ID NO: 2.

30. The composition of any one of claims 1-29, wherein the second polypeptide further comprises a sequence at least about 90%, 91%, 92%, 93%, 94%, 95%, 96%, 97%, 98%, 99%, or 100% identical to SEQ ID NO: 4.

31. The composition of any one of claims 1-31, wherein the second polypeptide comprises a sequence at least about 90%, 91%, 92%, 93%, 94%, 95%, 96%, 97%, 98%, 99%, or 100% identical to SEQ ID NO: 1.

32. The composition of any one of claims 1-31, wherein the first polypeptide and the second polypeptide are connected via one or more disulfide bonds.

33. The composition of any one of claims 1-32, wherein the first polypeptide and the second polypeptide form an antibody variable domain.

34. The composition of claim 33, wherein the antibody variable domain does not bind to an antigen with an equilibrium dissociation constant (K_{D}) lower than about 10⁻² M, 10⁻³ M, or 10⁻⁴ M.

35. The composition of claim 33 or claim 34, wherein the antibody variable domain comprises a modified palivizumab variable domain.

36. The composition of claim 35, wherein the modified palivizumab variable domain comprises a heavy chain CDR1 comprising SEQ ID NO: 19.

37. The composition of claim 35 or claim 36, wherein the modified palivizumab variable domain comprises a heavy chain CDR2 comprising SEQ ID NO: 20.

38. The composition of any one of claims 35-37, wherein the modified palivizumab variable domain comprises a heavy chain CDR3 comprising SEQ ID NO: 21.

39. The composition of any one of claims 35-38, wherein the modified palivizumab variable domain comprises a light chain CDR1 comprising SEQ ID NO: 22.

40. The composition of any one of claims 35-39, wherein the modified palivizumab variable domain comprises a light chain CDR2 comprising SEQ ID NO: 23.

41. The composition of any one of claims 35-40, wherein the modified palivizumab variable domain comprises a light chain CDR3 comprising SEQ ID NO: 24, 77 or 16.

42. The composition of any one of claims 35-41, wherein the modified palivizumab variable domain does not bind to Respiratory Syncytial Virus (RSV) with a K_{D} lower than about 10⁻² M, 10⁻³ M, or 10⁻⁴ M.

43. The composition of any one of claims 1-42, further comprising a Fc region comprising reduced effector function as compared to human IgG1.

44. The composition of claim 43, wherein the human IgG1 comprises SEQ ID NO: 25.

45. The composition of claim 43 or claim 44, wherein the reduced effector function comprises reduced antibody-dependent cellular cytotoxicity (ADCC).

46. The composition of any one of claims 43-45, wherein the reduced effector function comprises reduced complement dependent cytotoxicity (CDC).

47. The composition of any one of claims 1-46, wherein the first polypeptide further comprises a sequence at least about 90%, 91%, 92%, 93%, 94%, 95%, 96%, 97%, 98%, 99%, or 100% identical to SEQ ID NO: 3; and/or the first polypeptide comprises a Fc region comprising a human IgG1 comprising E233P, L234V, L235A, ΔG236, A327G, A330S, P331S, per Kabat numbering.

48. A composition comprising an antibody variable domain comprising a light chain sequence comprising a first polypeptide comprising a sequence at least about 90% identical to SEQ ID NO: 6, and a heavy chain sequence comprising a second polypeptide comprising a sequence at least about 90% identical to SEQ ID NO: 2.

49. The composition of claim 48, wherein the first polypeptide comprises a sequence at least about 91%, 92%, 93%, 94%, 95%, 96%, 97%, 98%, 99%, or 100% identical to SEQ ID NO: 6.

50. The composition of claim 48 or claim 49, wherein the second polypeptide comprises a sequence at least about 91%, 92%, 93%, 94%, 95%, 96%, 97%, 98%, 99%, or 100% identical to SEQ ID NO: 2.

51. The composition of any one of claims 48-50, comprising GM-CSF.

52. The composition of claim 51, wherein the GM-CSF is human GM-CSF or murine GM-CSF.

53. The composition of claim 51 or claim 52, wherein GM-CSF comprises a sequence at least about 85%, 86%, 87%, 88%, 89%, 90%, 91%, 92%, 93%, 94%, 95%, 96%, 97%, 98%, or 99% identical to SEQ ID NO: 16 or 77.

54. The composition of any one of claims 48-53, wherein the light chain comprises a sequence at least about 90%, 91%, 92%, 93%, 94%, 95%, 96%, 97%, 98%, 99%, or 100% identical to SEQ ID NO: 7.

55. The composition of any one of claims 48-54, wherein the light chain comprises a sequence at least about 90%, 91%, 92%, 93%, 94%, 95%, 96%, 97%, 98%, 99%, or 100% identical to SEQ ID NO: 5.

56. The composition of any one of claims 48-55, wherein the heavy chain comprises a sequence at least about 90%, 91%, 92%, 93%, 94%, 95%, 96%, 97%, 98%, 99%, or 100% identical to SEQ ID NO: 4.

57. The composition of any one of claims 48-56, wherein the heavy chain comprises a sequence at least about 90%, 91%, 92%, 93%, 94%, 95%, 96%, 97%, 98%, 99%, or 100% identical to SEQ ID NO: 1.

58. The composition of any one of claims 48-57, further comprising a Fc region comprising reduced effector function as compared to human IgG1.

59. The composition of claim 58, wherein the human IgG1 comprises SEQ ID NO: 25.

60. The composition of claim 58 or claim 59, wherein the reduced effector function comprises reduced antibody-dependent cellular cytotoxicity (ADCC).

61. The composition of any one of claims 58-60, wherein the reduced effector function comprises reduced complement dependent cytotoxicity (CDC).

62. The composition of any one of claims 48-61, wherein the heavy chain further comprises a sequence at least about 90%, 91%, 92%, 93%, 94%, 95%, 96%, 97%, 98%, 99%, or 100% identical to SEQ ID NO: 3; and/or the heavy chain comprises a Fc region comprising a human IgG1 comprising E233P, L234V, L235A, ΔG236, A327G, A330S, P331S, per Kabat numbering.

63. A composition comprising an antibody variable domain comprising a light chain sequence comprising a sequence at least about 90% identical to SEQ ID NO: 26 (DIQMTQSPSTLSASVGDRVTITCKCQLSVGYMHWYQQKPGKAPKLLIYDTSKLASGVPSRFS GSGSGTEFTLTISSLQPDDFATYYCFQGS[X1]PFTFGGGTKLEIKR), wherein the light chain sequence comprises X1 and X1 comprises GM-CSF; and a heavy chain sequence comprising a sequence at least about 90% identical to SEQ ID NO: 2.

64. The composition of claim 63, wherein the GM-CSF is human GM-CSF or murine GM-CSF.

65. The composition of claim 63 or claim 64, wherein GM-CSF comprises a sequence at least about 85%, 86%, 87%, 88%, 89%, 90%, 91%, 92%, 93%, 94%, 95%, 96%, 97%, 98%, or 99% identical to SEQ ID NO: 16 or 77.

66. The composition of any one of claims 63-65, wherein the light chain sequence comprises a sequence at least about 91%, 92%, 93%, 94%, 95%, 96%, 97%, 98%, 99%, or 100% identical to SEQ ID NO: 26.

67. The composition of any one of claims 63-66, wherein the light chain sequence comprises a sequence at least about 90%, 91%, 92%, 93%, 94%, 95%, 96%, 97%, 98%, 99%, or 100% identical to SEQ ID NO: 27 (DIQMTQSPSTLSASVGDRVTITCKCQLSVGYMHWYQQKPGKAPKLLIYDTSKLASGVPSRFS GSGSGTEFTLTISSLQPDDFATYYCFQGSGGSGAKLAALKAKLAALKGGGGS[X2]GGGGSEL AALEAELAALEAGGSGPFTFGGGTKLEIKR), wherein the light chain sequence comprises X2 and X2 comprises the GM-CSF.

68. The composition of any one of claims 63-67, wherein the heavy chain sequence comprises a sequence at least about 91%, 92%, 93%, 94%, 95%, 96%, 97%, 98%, 99%, or 100% identical to SEQ ID NO:

2.

69. The composition of any one of claims 63-68, wherein the light chain comprises a sequence at least about 90%, 91%, 92%, 93%, 94%, 95%, 96%, 97%, 98%, 99%, or 100% identical to SEQ ID NO: 7.

70. The composition of any one of claims 63-69, wherein the light chain comprises a sequence at least about 90%, 91%, 92%, 93%, 94%, 95%, 96%, 97%, 98%, 99%, or 100% identical to SEQ ID NO: 5.

71. The composition of any one of claims 63-70, wherein the heavy chain comprises a sequence at least about 90%, 91%, 92%, 93%, 94%, 95%, 96%, 97%, 98%, 99%, or 100% identical to SEQ ID NO: 4.

72. The composition of any one of claims 63-71, wherein the heavy chain comprises a sequence at least about 90%, 91%, 92%, 93%, 94%, 95%, 96%, 97%, 98%, 99%, or 100% identical to SEQ ID NO: 1.

73. The composition of any one of claims 63-72, further comprising a Fc region comprising reduced effector function as compared to human IgG1.

74. The composition of claim 73, wherein the human IgG1 comprises SEQ ID NO: 25.

75. The composition of claim 73 or claim 74, wherein the reduced effector function comprises reduced antibody-dependent cellular cytotoxicity (ADCC).

76. The composition of any one of claims 73-75, wherein the reduced effector function comprises reduced complement dependent cytotoxicity (CDC).

77. The composition of any one of claims 63-76, wherein the heavy chain further comprises a sequence at least about 90%, 91%, 92%, 93%, 94%, 95%, 96%, 97%, 98%, 99%, or 100% identical to SEQ ID NO: 3; and/or the heavy chain comprises a Fc region comprising a human IgG1 comprising E233P, L234V, L235A, ΔG236, A327G, A330S, P331S, per Kabat numbering.

78. A composition comprising a sequence at least about 90% identical to SEQ ID NO: 18.

79. The composition of claim 78, wherein the sequence is at least about 91%, 92%, 93%, 94%, 95%, 96%, 97%, 98%, 99%, or 100% identical to SEQ ID NO: 18.

80. The composition of claim 78 or claim 79, wherein the sequence is connected to an antibody domain.

81. The composition of claim 80, wherein the antibody domain is an antibody variable domain.

82. The composition of claim 80 or claim 81, wherein the sequence is positioned within the antibody domain.

83. The composition of claim 81, wherein the sequence is positioned within a CDR of the antibody variable domain.

84. The composition of claim 83, wherein the sequence is positioned within the CDR of a modified trastuzumab antibody variable domain.

85. The composition of claim 83, wherein the sequence is positioned within the CDR of a modified palivizumab antibody variable domain.

86. The composition of any one of claims 78-83, comprising a region at least about 90%, 91%, 92%, 93%, 94%, 95%, 96%, 97%, 98%, 99%, or 100% identical to SEQ ID NO: 42, wherein the region comprises the X5, and the X5 comprises the sequence.

87. The composition of claim 86, further comprising a region at least about 90%, 91%, 92%, 93%, 94%, 95%, 96%, 97%, 98%, 99%, or 100% identical to SEQ ID NO: 31.

88. The composition of any one of claims 78-83, comprising a region at least about 90%, 91%, 92%, 93%, 94%, 95%, 96%, 97%, 98%, 99%, or 100% identical to SEQ ID NO: 43, wherein the region comprises the X6, and the X6 comprises the sequence.

89. The composition of claim 88, further comprising a region at least about 90%, 91%, 92%, 93%, 94%, 95%, 96%, 97%, 98%, 99%, or 100% identical to SEQ ID NO: 2.

90. The composition of any one of claims 78-89, further comprising a Fc region comprising reduced effector function as compared to human IgG1.

91. The composition of claim 90, wherein the human IgG1 comprises SEQ ID NO: 25.

92. The composition of claim 90 or claim 91, wherein the reduced effector function comprises reduced antibody-dependent cellular cytotoxicity (ADCC).

93. The composition of any one of claims 90-92, wherein the reduced effector function comprises reduced complement dependent cytotoxicity (CDC).

94. The composition of any one of claims 90-93, wherein the Fc region comprises a sequence at least about 90%, 91%, 92%, 93%, 94%, 95%, 96%, 97%, 98%, 99%, or 100% identical to SEQ ID NO: 3; and/or the Fc region comprises a human IgG1 comprising E233P, L234V, L235A, ΔG236, A327G, A330S, P331S, per Kabat numbering.

95. A composition comprising a first polypeptide comprising: (i) SEQ ID NOS: 22, 23, and 16, and a second polypeptide comprising SEQ ID NOS: 19-21, or (ii) SEQ ID NOS: 22, 23, and 77, and a second polypeptide comprising SEQ ID NOS: 19-21.

96. The composition of claim 95, wherein the first polypeptide is a light chain of an antibody variable domain.

97. The composition of claim 95 or claim 96, wherein the second polypeptide is a heavy chain of an antibody variable domain.

98. The composition of any one of claims 95-97, wherein the first polypeptide comprises SEQ ID NO: 24.

99. The composition of any one of claims 95-98, further comprising a Fc region comprising reduced effector function as compared to human IgG1.

100. The composition of claim 99, wherein the human IgG1 comprises SEQ ID NO: 25.

101. The composition of claim 99 or claim 100, wherein the reduced effector function comprises reduced antibody-dependent cellular cytotoxicity (ADCC).

102. The composition of any one of claims 99-101, wherein the reduced effector function comprises reduced complement dependent cytotoxicity (CDC).

103. The composition of any one of claims 95-102, wherein the second polypeptide further comprises a sequence at least about 90%, 91%, 92%, 93%, 94%, 95%, 96%, 97%, 98%, 99%, or 100% identical to SEQ ID NO: 3; and/or the second polypeptide comprises a Fc region comprising a human IgG1 comprising E233P, L234V, L235A, ΔG236, A327G, A330S, P331S, per Kabat numbering.

104. A composition comprising a first polypeptide comprising: (i) SEQ ID NOS: 37-39, and a second polypeptide comprising SEQ ID NOS: 34, 35, 16, or (ii) SEQ ID NOS: 37-39, and a second polypeptide comprising SEQ ID NOS: 34, 35, 77.

105. The composition of claim 104, wherein the first polypeptide is a light chain of an antibody variable domain.

106. The composition of claim 104 or claim 105, wherein the second polypeptide is a heavy chain of an antibody variable domain.

107. The composition of any one of claims 104-106, wherein the first polypeptide comprises SEQ ID NO: 36.

108. The composition of any one of claims 104-107, further comprising a Fc region comprising reduced effector function as compared to human IgG1.

109. The composition of claim 108, wherein the human IgG1 comprises SEQ ID NO: 25.

110. The composition of claim 108 or claim 109, wherein the reduced effector function comprises reduced antibody-dependent cellular cytotoxicity (ADCC).

111. The composition of any one of claims 108-110, wherein the reduced effector function comprises reduced complement dependent cytotoxicity (CDC).

112. The composition of any one of claims 104-111, wherein the second polypeptide further comprises a sequence at least about 90%, 91%, 92%, 93%, 94%, 95%, 96%, 97%, 98%, 99%, or 100% identical to SEQ ID NO: 3; and/or the second polypeptide comprises a Fc region comprising a human IgG1 comprising E233P, L234V, L235A, ΔG236, A327G, A330S, P331S, per Kabat numbering.

113. A composition comprising a first polypeptide comprising SEQ ID NO: 31, and a second polypeptide comprising a granulocyte macrophage colony stimulating factor (GM-CSF).

114. The composition of claim 113, wherein the GM-CSF comprises a sequence at least about 85%, 86%, 87%, 88%, 89%, 90%, 91%, 92%, 93%, 94%, 95%, 96%, 97%, 98%, or 99% identical to SEQ ID NO: 16 or 77.

115. The composition of claim 113 or claim 114, wherein the GM-CSF comprises human GM-CSF or murine GM-CSF.

116. The composition of any one of claims 113-115, wherein the second polypeptide comprises a modified heavy chain of an antibody variable region.

117. The composition of claim 116, wherein the modified heavy chain of the antibody variable domain comprises the GM-CSF positioned between a first amino acid sequence of the antibody variable region and a second amino acid sequence of the antibody variable region.

118. The composition of claim 117, wherein the first amino acid sequence comprises a sequence at least about 90%, 91%, 92%, 93%, 94%, 95%, 96%, 97%, 98%, or 99% identical to SEQ ID NO: 32.

119. The composition of claim 118, wherein the first amino acid sequence comprises SEQ ID NO: 32.

120. The composition of any one of claims 117-119, wherein the second amino acid sequence comprises a sequence at least about 90%, 91%, 92%, 93%, 94%, 95%, 96%, 97%, 98%, or 99% identical to SEQ ID NO: 33.

121. The composition of claim 120, wherein the second amino acid sequence comprises SEQ ID NO: 33.

122. The composition of any one of claims 116-121, wherein the GM-CSF is positioned within a complementarity determining region (CDR) of the modified heavy chain.

123. The composition of claim 122, wherein the GM-CSF is position within heavy chain CDR1, CDR2, or CDR3.

124. The composition of claim 123, wherein the GM-CSF is positioned within heavy chain CDR3.

125. The composition of any one of claims 116-124, wherein the modified heavy chain is modified from a variable heavy chain comprising SEQ ID NO: 44.

126. The composition of any one of claims 113-125, wherein the second polypeptide further comprises a first linker peptide.

127. The composition of claim 126, wherein the first linker peptide comprises SEQ ID NO: 10.

128. The composition of claim 126 or claim 127, wherein the first linker peptide comprises SEQ ID NO: 8.

129. The composition of any one of claims 126-128, wherein the first linker peptide comprises SEQ ID NO: 11.

130. The composition of any one of claims 126-129, wherein the first linker peptide comprises a sequence at least about 90%, 91%, 92%, 93%, 94%, 95%, 96%, 97%, 98%, 99% or 100% identical to SEQ ID NO: 12.

131. The composition of any one of claims 113-130, wherein the second polypeptide further comprises a second linker peptide.

132. The composition of claim 131, wherein the second linker peptide comprises SEQ ID NO: 10.

133. The composition of claim 131 or claim 132, wherein the second linker peptide comprises SEQ ID NO: 9.

134. The composition of any one of claims 131-133, wherein the second linker peptide comprises SEQ ID NO: 11.

135. The composition of any one of claims 131-134, wherein the second linker peptide comprises a sequence at least about 90%, 91%, 92%, 93%, 94%, 95%, 96%, 97%, 98%, 99%, or 100% identical to SEQ ID NO: 13.

136. The composition of any one of claims 113-135, wherein the second polypeptide comprises a sequence at least about 90%, 91%, 92%, 93%, 94%, 95%, 96%, 97%, 98%, 99%, or 100% identical to SEQ ID NO: 18.

137. The composition of any one of claims 113-136, wherein the second polypeptide comprises a sequence at least about 90%, 91%, 92%, 93%, 94%, 95%, 96%, 97%, 98%, 99%, or 100% identical to SEQ ID NO: 29.

138. The composition of any one of claims 113-137, wherein the second polypeptide comprises a sequence at least about 90%, 91%, 92%, 93%, 94%, 95%, 96%, 97%, 98%, 99%, or 100% identical to SEQ ID NO: 4.

139. The composition of any one of claims 113-138, wherein the second polypeptide comprises a sequence at least about 90%, 91%, 92%, 93%, 94%, 95%, 96%, 97%, 98%, 99%, or 100% identical to SEQ ID NO: 28.

140. The composition of any one of claims 113-139, wherein the first polypeptide comprises a light chain of an antibody variable region.

141. The composition of any one of claims 113-140, wherein the first polypeptide comprises SEQ ID NO: 31.

142. The composition of any one of claims 113-141, wherein the first polypeptide further comprises a sequence at least about 90%, 91%, 92%, 93%, 94%, 95%, 96%, 97%, 98%, 99%, or 100% identical to SEQ ID NO: 7.

143. The composition of any one of claims 113-142, wherein the second polypeptide comprises a sequence at least about 90%, 91%, 92%, 93%, 94%, 95%, 96%, 97%, 98%, 99%, or 100% identical to SEQ ID NO: 30.

144. The composition of any one of claims 113-143, wherein the first polypeptide and the second polypeptide are connected via one or more disulfide bonds.

145. The composition of any one of claims 113-144, wherein the first polypeptide and the second polypeptide form an antibody variable domain.

146. The composition of claim 145, wherein the antibody variable domain does not bind to an antigen with an equilibrium dissociation constant (K_{D}) lower than about 10⁻² M, 10⁻³ M, or 10⁻⁴ M.

147. The composition of claim 145 or claim 146, wherein the antibody variable domain comprises a modified trastuzumab variable domain.

148. The composition of claim 147, wherein the modified trastuzumab variable domain comprises a heavy chain CDR1 comprising SEQ ID NO: 34.

149. The composition of claim 147 or claim 148, wherein the modified trastuzumab variable domain comprises a heavy chain CDR2 comprising SEQ ID NO: 35.

150. The composition of any one of claims 147-149, wherein the modified trastuzumab variable domain comprises a heavy chain CDR3 comprising SEQ ID NO: 36, 77 or 16.

151. The composition of any one of claims 147-150, wherein the modified trastuzumab variable domain comprises a light chain CDR1 comprising SEQ ID NO: 37.

152. The composition of any one of claims 147-151, wherein the modified trastuzumab variable domain comprises a light chain CDR2 comprising SEQ ID NO: 38.

153. The composition of any one of claims 147-152, wherein the modified trastuzumab variable domain comprises a light chain CDR3 comprising SEQ ID NO: 39.

154. The composition of any one of claims 147-153, wherein the modified trastuzumab variable domain does not bind to human epidermal growth factor receptor 2 (Her2) with a K_{D} lower than about 10⁻² M, 10⁻³ M, or 10⁻⁴ M.

155. The composition of any one of claims 113-154, further comprising a Fc region comprising reduced effector function as compared to human IgG1.

156. The composition of claim 155, wherein the human IgG1 comprises SEQ ID NO: 25.

157. The composition of claim 155 or claim 156, wherein the reduced effector function comprises reduced antibody-dependent cellular cytotoxicity (ADCC).

158. The composition of any one of claims 155-157, wherein the reduced effector function comprises reduced complement dependent cytotoxicity (CDC).

159. The composition of any one of claims 113-158, wherein the second polypeptide further comprises a sequence at least about 90%, 91%, 92%, 93%, 94%, 95%, 96%, 97%, 98%, 99%, or 100% identical to SEQ ID NO: 3; and/or the second polypeptide comprises a Fc region comprising a human IgG1 comprising E233P, L234V, L235A, ΔG236, A327G, A330S, P331S, per Kabat numbering.

160. A composition comprising an antibody variable domain comprising a light chain sequence comprising a first polypeptide comprising a sequence at least about 90% identical to SEQ ID NO: 31, and a heavy chain sequence comprising a second polypeptide comprising a sequence at least about 90% identical to SEQ ID NO: 29.

161. The composition of claim 160, wherein the first polypeptide comprises a sequence at least about 91%, 92%, 93%, 94%, 95%, 96%, 97%, 98%, 99%, or 100% identical to SEQ ID NO: 31.

162. The composition of claim 160 or claim 161, wherein the second polypeptide comprises a sequence at least about 91%, 92%, 93%, 94%, 95%, 96%, 97%, 98%, 99%, or 100% identical to SEQ ID NO: 29.

163. The composition of any one of claims 160-162, comprising GM-CSF.

164. The composition of claim 163, wherein the GM-CSF is human GM-CSF or murine GM-CSF.

165. The composition of claim 163 or claim 164, wherein GM-CSF comprises a sequence at least about 85%, 86%, 87%, 88%, 89%, 90%, 91%, 92%, 93%, 94%, 95%, 96%, 97%, 98%, or 99% identical to SEQ ID NO: 16 or 77.

166. The composition of any one of claims 160-165, wherein the light chain comprises a sequence at least about 90%, 91%, 92%, 93%, 94%, 95%, 96%, 97%, 98%, 99%, or 100% identical to SEQ ID NO: 7.

167. The composition of any one of claims 160-166, wherein the light chain comprises a sequence at least about 90%, 91%, 92%, 93%, 94%, 95%, 96%, 97%, 98%, 99%, or 100% identical to SEQ ID NO: 30.

168. The composition of any one of claims 160-167, wherein the heavy chain comprises a sequence at least about 90%, 91%, 92%, 93%, 94%, 95%, 96%, 97%, 98%, 99%, or 100% identical to SEQ ID NO: 4.

169. The composition of any one of claims 160-168, wherein the heavy chain comprises a sequence at least about 90%, 91%, 92%, 93%, 94%, 95%, 96%, 97%, 98%, 99%, or 100% identical to SEQ ID NO: 28.

170. The composition of any one of claims 160-169, further comprising a Fc region comprising reduced effector function as compared to human IgG1.

171. The composition of claim 170, wherein the human IgG1 comprises SEQ ID NO: 25.

172. The composition of claim 170 or claim 171, wherein the reduced effector function comprises reduced antibody-dependent cellular cytotoxicity (ADCC).

173. The composition of any one of claims 170-172, wherein the reduced effector function comprises reduced complement dependent cytotoxicity (CDC).

174. The composition of any one of claims 160-173, wherein the heavy chain further comprises a sequence at least about 90%, 91%, 92%, 93%, 94%, 95%, 96%, 97%, 98%, 99%, or 100% identical to SEQ ID NO: 3; and/or the heavy chain comprises a Fc region comprising a human IgG1 comprising E233P, L234V, L235A, ΔG236, A327G, A330S, P331S, per Kabat numbering.

175. A composition comprising an antibody variable domain comprising a heavy chain sequence comprising a sequence at least about 90% identical to SEQ ID NO: 42 (EVQLVESGGGLVQPGGSLRLSCAASGFNIKDTYIHWVRQAPGKGLEWVARIYPTNGYTRYA DSVKGRFTISADTSKNTAYLQMNSLRAEDTAVYYCSR[[X5]]WGQGTLVTVSS), wherein the heavy chain sequence comprises X6 and X6 comprises GM-CSF; and a light chain sequence comprising a sequence at least about 90% identical to SEQ ID NO: 31.

176. The composition of claim 175, wherein the GM-CSF is human GM-CSF or murine GM-CSF.

177. The composition of claim 175 or claim 176, wherein GM-CSF comprises a sequence at least about 85%, 86%, 87%, 88%, 89%, 90%, 91%, 92%, 93%, 94%, 95%, 96%, 97%, 98%, or 99% identical to SEQ ID NO: 16 or 77.

178. The composition of any one of claims 175-177, wherein the heavy chain sequence comprises a sequence at least about 91%, 92%, 93%, 94%, 95%, 96%, 97%, 98%, 99%, or 100% identical to SEQ ID NO: 42.

179. The composition of any one of claims 175-178, wherein the heavy chain sequence comprises a sequence at least about 90%, 91%, 92%, 93%, 94%, 95%, 96%, 97%, 98%, 99%, or 100% identical to SEQ ID NO: 43 (EVQLVESGGGLVQPGGSLRLSCAASGFNIKDTYIHWVRQAPGKGLEWVARIYPTNGYTRYA DSVKGRFTISADTSKNTAYLQMNSLRAEDTAVYYCSRGGSGAKLAALKAKLAALKGGGGS [[X6]]GGGGSELAALEAELAALEAGGSGDYWGQGTLVTVSS), wherein the heavy chain sequence comprises X6 and X6 comprises the GM-CSF.

180. The composition of any one of claims 175-179, wherein the heavy chain sequence comprises a sequence at least about 91%, 92%, 93%, 94%, 95%, 96%, 97%, 98%, 99%, or 100% identical to SEQ ID NO: 43.

181. The composition of any one of claims 175-180, wherein the light chain comprises a sequence at least about 90%, 91%, 92%, 93%, 94%, 95%, 96%, 97%, 98%, 99%, or 100% identical to SEQ ID NO: 7.

182. The composition of any one of claims 175-181, wherein the heavy chain comprises a sequence at least about 90%, 91%, 92%, 93%, 94%, 95%, 96%, 97%, 98%, 99%, or 100% identical to SEQ ID NO: 29.

183. The composition of any one of claims 175-182, wherein the heavy chain comprises a sequence at least about 90%, 91%, 92%, 93%, 94%, 95%, 96%, 97%, 98%, 99%, or 100% identical to SEQ ID NO: 4.

184. The composition of any one of claims 175-183, wherein the heavy chain comprises a sequence at least about 90%, 91%, 92%, 93%, 94%, 95%, 96%, 97%, 98%, 99%, or 100% identical to SEQ ID NO: 28.

185. The composition of any one of claims 175-184, further comprising a Fc region comprising reduced effector function as compared to human IgG1.

186. The composition of claim 185, wherein the human IgG1 comprises SEQ ID NO: 25.

187. The composition of claim 185 or claim 186, wherein the reduced effector function comprises reduced antibody-dependent cellular cytotoxicity (ADCC).

188. The composition of any one of claims 185-187, wherein the reduced effector function comprises reduced complement dependent cytotoxicity (CDC).

189. The composition of any one of claims 175-188, wherein the heavy chain further comprises a sequence at least about 90%, 91%, 92%, 93%, 94%, 95%, 96%, 97%, 98%, 99%, or 100% identical to SEQ ID NO: 3; and/or the heavy chain comprises a Fc region comprising a human IgG1 comprising E233P, L234V, L235A, ΔG236, A327G, A330S, P331S, per Kabat numbering.

190. Use of the compositions of any one of claims 1-189, for the treatment of a neurological disease or condition.

191. A method of treating a neurological disease or condition, comprising administering to a subject in need thereof a composition comprising any one of claims 1-189.

192. The use of claim 190 or the method of claim 191, wherein the neurological disease or condition comprises Parkinson's disease.

193. Use of the compositions of any one of claims 1-189, for the treatment of Alzheimer's disease and/or traumatic brain injury.

194. Use of the compositions of any one of claims 1-189, for the treatment of ALS.

195. Use of the compositions of any one of claims 1-189, for the treatment of acute radiation syndrome.

196. Use of the compositions of any one of claims 1-189, for the treatment of cancer.

197. The use or method of any one of claims 190-196, wherein the composition is administered once every about 7, 8, 9, 10, 11, 12, 13, 14, 15, 16, 17, 18, 19, 20, 21, 22, 23, 24, 25, 26, 27, 28, 29, or 30 days during a treatment period.

198. The use or method of claim 196, wherein the composition is administered once every about 14 days during a treatment period.

199. The use or method of any one of claims 190-196, wherein the composition is administered once every about 2 weeks during a treatment period.

200. The use or method of any one of claims 190-196, wherein the composition is administered once every about 3 weeks during a treatment period.

201. The use or method of any one of claims 190-196, wherein the composition is administered once every about 4 weeks during a treatment period.

202. The use or method of any one of claims 190-196, wherein the composition is administered about once a month during a treatment period.

203. The use or method of any one of claims 197-202, wherein the treatment period comprises from about 8 weeks to about 2 years.


SEQUENCE LISTING (77 sequences)

SEQ ID NO: 1 - protein, 449 aa, Artificial Sequence
  Description of Artificial Sequence Synthetic polypeptide
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITFGGFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 120 aa, Artificial Sequence
  Description of Artificial Sequence Synthetic polypeptide
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITFGGFDVWGAGTTVTVSS

SEQ ID NO: 3 - protein, 226 aa, Artificial Sequence
  Description of Artificial Sequence Synthetic polypeptide
DKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 103 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 5 - protein, 384 aa, Artificial Sequence
  Description of Artificial Sequence Synthetic polypeptide
DIQMTQSPSTLSASVGDRVTITCKCQLSVGYMHWYQQKPGKAPKLLIYDTSKLASGVPSRFSGSGSGTEFTLTISSLQPDDFATYYCFQGSGGSGAKLAALKAKLAALKGGGGSAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGSELAALEAELAALEAGGSGPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 278 aa, Artificial Sequence
  Description of Artificial Sequence Synthetic polypeptide
DIQMTQSPSTLSASVGDRVTITCKCQLSVGYMHWYQQKPGKAPKLLIYDTSKLASGVPSRFSGSGSGTEFTLTISSLQPDDFATYYCFQGSGGSGAKLAALKAKLAALKGGGGSAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGSELAALEAELAALEAGGSGPFTFGGGTKLEIKR

SEQ ID NO: 7 - protein, 106 aa, Artificial Sequence
  Description of Artificial Sequence Synthetic polypeptide
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 14 aa, Artificial Sequence
  Description of Artificial Sequence Synthetic peptide
AKLAALKAKLAALK

SEQ ID NO: 9 - protein, 14 aa, Artificial Sequence
  Description of Artificial Sequence Synthetic peptide
ELAALEAELAALEA

SEQ ID NO: 10 - protein, 4 aa, Artificial Sequence
  Description of Artificial Sequence Synthetic peptide
GGSG

SEQ ID NO: 11 - protein, 5 aa, Artificial Sequence
  Description of Artificial Sequence Synthetic peptide
GGGGS

SEQ ID NO: 12 - protein, 23 aa, Artificial Sequence
  Description of Artificial Sequence Synthetic peptide
GGSGAKLAALKAKLAALKGGGGS

SEQ ID NO: 13 - protein, 23 aa, Artificial Sequence
  Description of Artificial Sequence Synthetic peptide
GGGGSELAALEAELAALEAGGSG

SEQ ID NO: 14 - protein, 23 aa, Artificial Sequence
  Description of Artificial Sequence Synthetic peptide
DIQMTQSPSTLSASVGDRVTITC

SEQ ID NO: 15 - protein, 10 aa, Artificial Sequence
  Description of Artificial Sequence Synthetic peptide
FGGGTKLEIK

SEQ ID NO: 16 - protein, 127 aa, Homo sapiens [sequence omitted]

SEQ ID NO: 17 - protein, 107 aa, Artificial Sequence
  Description of Artificial Sequence Synthetic polypeptide
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKR

SEQ ID NO: 18 - protein, 173 aa, Artificial Sequence
  Description of Artificial Sequence Synthetic polypeptide
GGSGAKLAALKAKLAALKGGGGSAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGSELAALEAELAALEAGGSG

SEQ ID NO: 19 - protein, 12 aa, Artificial Sequence
  Description of Artificial Sequence Synthetic peptide
GFSLSTSGMSVG

SEQ ID NO: 20 - protein, 16 aa, Artificial Sequence
  Description of Artificial Sequence Synthetic peptide
DIWWDDKKDYNPSLKS

SEQ ID NO: 21 - protein, 10 aa, Artificial Sequence
  Description of Artificial Sequence Synthetic peptide
SMITFGGFDV

SEQ ID NO: 22 - protein, 10 aa, Artificial Sequence
  Description of Artificial Sequence Synthetic peptide
KCQLSVGYMH

SEQ ID NO: 23 - protein, 7 aa, Artificial Sequence
  Description of Artificial Sequence Synthetic peptide
DTSKLAS

SEQ ID NO: 24 - protein, 180 aa, Artificial Sequence
  Description of Artificial Sequence Synthetic polypeptide
FQGSGGSGAKLAALKAKLAALKGGGGSAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGSELAALEAELAALEAGGSGPFT

SEQ ID NO: 25 - protein, 330 aa, Homo sapiens [sequence omitted]

SEQ ID NO: 26 - protein, 105 aa, Artificial Sequence
  Description of Artificial Sequence Synthetic polypeptide
  NON_CONS (91)..(92): Residues at these positions can be separated by a GM-CSF sequence of unknown length
  See specification as filed for detailed description of substitutions and preferred embodiments
DIQMTQSPSTLSASVGDRVTITCKCQLSVGYMHWYQQKPGKAPKLLIYDTSKLASGVPSRFSGSGSGTEFTLTISSLQPDDFATYYCFQGSPFTFGGGTKLEIKR

SEQ ID NO: 27 - protein, 151 aa, Artificial Sequence
  Description of Artificial Sequence Synthetic polypeptide
  NON_CONS (114)..(115): Residues at these positions can be separated by a GM-CSF sequence of unknown length
  See specification as filed for detailed description of substitutions and preferred embodiments
DIQMTQSPSTLSASVGDRVTITCKCQLSVGYMHWYQQKPGKAPKLLIYDTSKLASGVPSRFSGSGSGTEFTLTISSLQPDDFATYYCFQGSGGSGAKLAALKAKLAALKGGGGSGGGGSELAALEAELAALEAGGSGPFTFGGGTKLEIKR

SEQ ID NO: 28 - protein, 613 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRGGSGAKLAALKAKLAALKGGGGSAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGSELAALEAELAALEAGGSGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 284 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRGGSGAKLAALKAKLAALKGGGGSAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGSELAALEAELAALEAGGSGDYWGQGTLVTVSS

SEQ ID NO: 30 - protein, 214 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 108 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKR

SEQ ID NO: 32 - protein, 25 aa, Artificial Sequence [sequence omitted]

SEQ ID NO: 33 - protein, 11 aa, Artificial Sequence
  Description of Artificial Sequence Synthetic peptide
WGQGTLVTVSS

SEQ ID NO: 34 - protein, 10 aa, Artificial Sequence
  Description of Artificial Sequence Synthetic peptide
GFNIKDTYIH

SEQ ID NO: 35 - protein, 17 aa, Artificial Sequence
  Description of Artificial Sequence Synthetic peptide
RIYPTNGYTRYADSVKG

SEQ ID NO: 36 - protein, 175 aa, Artificial Sequence
  Description of Artificial Sequence Synthetic polypeptide
GGSGAKLAALKAKLAALKGGGGSAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGSELAALEAELAALEAGGSGDY

SEQ ID NO: 37 - protein, 11 aa, Artificial Sequence
  Description of Artificial Sequence Synthetic peptide
RASQDVNTAVA

SEQ ID NO: 38 - protein, 7 aa, Artificial Sequence
  Description of Artificial Sequence Synthetic peptide
SASFLYS

SEQ ID NO: 39 - protein, 9 aa, Artificial Sequence
  Description of Artificial Sequence Synthetic peptide
QQHYTTPPT

SEQ ID NO: 40 - protein, 111 aa, Artificial Sequence
  Description of Artificial Sequence Synthetic polypeptide
  NON_CONS (98)..(99): Residues at these positions can be separated by a GM-CSF sequence of unknown length
  See specification as filed for detailed description of substitutions and preferred embodiments
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRDYWGQGTLVTVSS

SEQ ID NO: 41 - protein, 157 aa, Artificial Sequence
  Description of Artificial Sequence Synthetic polypeptide
  NON_CONS (121)..(122): Residues at these positions can be separated by a GM-CSF sequence of unknown length
  See specification as filed for detailed description of substitutions and preferred embodiments
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRGGSGAKLAALKAKLAALKGGGGSGGGGSELAALEAELAALEAGGSGDYWGQGTLVTVSS

SEQ ID NO: 42 - protein, 109 aa, Artificial Sequence
  Description of Artificial Sequence Synthetic polypeptide
  NON_CONS (98)..(99): Residues at these positions can be separated by a GM-CSF sequence of unknown length
  See specification as filed for detailed description of substitutions and preferred embodiments
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGQGTLVTVSS

SEQ ID NO: 43 - protein, 157 aa, Artificial Sequence; identical to SEQ ID NO: 41
  Description of Artificial Sequence Synthetic polypeptide
  NON_CONS (121)..(122): Residues at these positions can be separated by a GM-CSF sequence of unknown length
  See specification as filed for detailed description of substitutions and preferred embodiments

SEQ ID NO: 44 - protein, 120 aa, Artificial Sequence [sequence omitted]

SEQ ID NO: 45 - protein, 263 aa, Artificial Sequence
  Description of Artificial Sequence Synthetic polypeptide
APARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGSGGGGSGGGGSGQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITFGGFDVWGAGTTVTVSS

SEQ ID NO: 46 - protein, 107 aa, Artificial Sequence
  Description of Artificial Sequence Synthetic polypeptide
DIQMTQSPSTLSASVGDRVTITCKCQLSVGYMHWYQQKPGKAPKLLIYDTSKLASGVPSRFSGSGSGTAFTLTISSLQPDDFATYYCFQGNGYPFTFGGGTKLEIKR

SEQ ID NO: 47 - protein, 592 aa, Artificial Sequence
  Description of Artificial Sequence Synthetic polypeptide
APARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGSGGGGSGGGGSGQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITFGGFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 213 aa, Artificial Sequence
  Description of Artificial Sequence Synthetic polypeptide
DIQMTQSPSTLSASVGDRVTITCKCQLSVGYMHWYQQKPGKAPKLLIYDTSKLASGVPSRFSGSGSGTAFTLTISSLQPDDFATYYCFQGNGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 260 aa, Artificial Sequence
  Description of Artificial Sequence Synthetic polypeptide
APTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQKGGGGSGGGGSGGGGSGQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITFGGFDVWGAGTTVTVSS

SEQ ID NO: 50 - protein, 107 aa, Artificial Sequence; identical to SEQ ID NO: 46
  Description of Artificial Sequence Synthetic polypeptide

SEQ ID NO: 51 - protein, 589 aa, Artificial Sequence
  Description of Artificial Sequence Synthetic polypeptide
APTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQKGGGGSGGGGSGGGGSGQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITFGGFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 213 aa, Artificial Sequence; identical to SEQ ID NO: 48
  Description of Artificial Sequence Synthetic polypeptide

SEQ ID NO: 53 - protein, 281 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRGGSGAKLAALKAKLAALKGGGGSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQKGGGGSELAALEAELAALEAGGSGDYWGQGTLVTVSS

SEQ ID NO: 54 - protein, 108 aa, Artificial Sequence; identical to SEQ ID NO: 31
  Description of Artificial Sequence Synthetic polypeptide

SEQ ID NO: 55 - protein, 610 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRGGSGAKLAALKAKLAALKGGGGSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQKGGGGSELAALEAELAALEAGGSGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 214 aa, Artificial Sequence; identical to SEQ ID NO: 30
  Description of Artificial Sequence Synthetic polypeptide

SEQ ID NO: 57 - protein, 120 aa, Artificial Sequence; identical to SEQ ID NO: 2
  Description of Artificial Sequence Synthetic polypeptide

SEQ ID NO: 58 - protein, 275 aa, Artificial Sequence
  Description of Artificial Sequence Synthetic polypeptide
DIQMTQSPSTLSASVGDRVTITCKCQLSVGYMHWYQQKPGKAPKLLIYDTSKLASGVPSRFSGSGSGTAFTLTISSLQPDDFATYYCFQGNGGSGAKLAALKAKLAALKGGGGSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQKGGGGSELAALEAELAALEAGGSGPFTFGGGTKLEIKR

SEQ ID NO: 59 - protein, 449 aa, Artificial Sequence; identical to SEQ ID NO: 1
  Description of Artificial Sequence Synthetic polypeptide

SEQ ID NO: 60 - protein, 381 aa, Artificial Sequence
  Description of Artificial Sequence Synthetic polypeptide
DIQMTQSPSTLSASVGDRVTITCKCQLSVGYMHWYQQKPGKAPKLLIYDTSKLASGVPSRFSGSGSGTAFTLTISSLQPDDFATYYCFQGNGGSGAKLAALKAKLAALKGGGGSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQKGGGGSELAALEAELAALEAGGSGPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 120 aa, Artificial Sequence; identical to SEQ ID NO: 2
  Description of Artificial Sequence Synthetic polypeptide

SEQ ID NO: 62 - protein, 250 aa, Artificial Sequence
  Description of Artificial Sequence Synthetic polypeptide
APARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGSGGGGSGGGGSGDIQMTQSPSTLSASVGDRVTITCKCQLSVGYMHWYQQKPGKAPKLLIYDTSKLASGVPSRFSGSGSGTAFTLTISSLQPDDFATYYCFQGNGYPFTFGGGTKLEIKR

SEQ ID NO: 63 - protein, 449 aa, Artificial Sequence; identical to SEQ ID NO: 1
  Description of Artificial Sequence Synthetic polypeptide

SEQ ID NO: 64 - protein, 356 aa, Artificial Sequence
  Description of Artificial Sequence Synthetic polypeptide
APARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGSGGGGSGGGGSGDIQMTQSPSTLSASVGDRVTITCKCQLSVGYMHWYQQKPGKAPKLLIYDTSKLASGVPSRFSGSGSGTAFTLTISSLQPDDFATYYCFQGNGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 120 aa, Artificial Sequence
  Description of Artificial Sequence Synthetic polypeptide
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSS

SEQ ID NO: 66 - protein, 10 aa, Artificial Sequence
  Description of Artificial Sequence Synthetic peptide
  MOD_RES (5)..(5): F, A, G or P
  MOD_RES (6)..(6): G, A, S, T or P
  MOD_RES (7)..(7): G, A, V, L or P
SMITXXXFDV

SEQ ID NO: 67 - protein, 374 aa, Artificial Sequence
  Description of Artificial Sequence Synthetic polypeptide
APARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGSGGSGGGGSGGGGSEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 371 aa, Artificial Sequence
  Description of Artificial Sequence Synthetic polypeptide
APTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQKGGSGGSGGGGSGGGGSEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - DNA, 1839 bp, Artificial Sequence
  Description of Artificial Sequence Synthetic polynucleotide
gaggtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagaggcggaagcggagcaaagctcgccgcactgaaagccaagctggccgctctgaagggaggtggcgggagcgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagggcggaggtgggagtgaactggccgcactggaagctgagctggctgccctcgaagctggaggctctggagactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 70 - DNA, 642 bp, Artificial Sequence
  Description of Artificial Sequence Synthetic polynucleotide
gacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaaggtggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 71 - DNA, 1347 bp, Artificial Sequence
  Description of Artificial Sequence Synthetic polynucleotide
caggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccttcgggggcttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 72 - DNA, 1152 bp, Artificial Sequence
  Description of Artificial Sequence Synthetic polynucleotide
gacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggcggaagcggagcaaagctcgccgcactgaaagccaagctggccgctctgaagggaggtggcgggagcgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagggcggaggtgggagtgaactggccgcactggaagctgagctggctgccctcgaagctggaggctctggacccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 73 - DNA, 852 bp, Artificial Sequence
  Description of Artificial Sequence Synthetic polynucleotide
gaggtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagaggcggaagcggagcaaagctcgccgcactgaaagccaagctggccgctctgaagggaggtggcgggagcgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagggcggaggtgggagtgaactggccgcactggaagctgagctggctgccctcgaagctggaggctctggagactactggggccaaggaaccctggtcaccgtctcctca

SEQ ID NO: 74 - DNA, 324 bp, Artificial Sequence
  Description of Artificial Sequence Synthetic polynucleotide
gacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaaggtggagatcaaacga

SEQ ID NO: 75 - DNA, 360 bp, Artificial Sequence
  Description of Artificial Sequence Synthetic polynucleotide
caggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccttcgggggcttcgacgtgtggggagccggtaccaccgtgaccgtgtcttcc

SEQ ID NO: 76 - DNA, 834 bp, Artificial Sequence
  Description of Artificial Sequence Synthetic polynucleotide
gacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggcggaagcggagcaaagctcgccgcactgaaagccaagctggccgctctgaagggaggtggcgggagcgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagggcggaggtgggagtgaactggccgcactggaagctgagctggctgccctcgaagctggaggctctggacccttcaccttcggcggcggcaccaagctggagatcaaacga

SEQ ID NO: 77 - protein, 34 aa, Artificial Sequence
  Description of Artificial Sequence Synthetic polypeptide
INVKCSLPQQCIKPCKDAGMRFGKCMNKKCRCYS